(19) United States
(12) Patent Application Publication
(10) Pub. No.: US 2020/0188476 A1
(43) Pub. Date: Jun. 18, 2020

(54) ANTI-MICROBIAL AGENT-POLYMER CONJUGATES AND METHODS OF USE THEREOF

(71) Applicant: The Regents of the University of California, Oakland, CA (US)

(72) Inventors: Niren Murthy, Berkeley, CA (US); Jingtuo Zhang, Berkeley, CA (US); Giri K. Vegesna, South San Francisco, CA (US); Bora Park, Berkeley, CA (US)

(21) Appl. No.: 16/500,701

(22) PCT Filed: Apr. 17, 2018
(86) PCT No.: PCT/US2018/027973

§ 371 Date: Oct. 3, 2019

Related U.S. Application Data
(60) Provisional application No. 62/486,818, filed on Apr. 18, 2017.

(51) Int. Cl. A61K 38/12; A61K 47/61; A61P 31/04

(52) U.S. Cl. CPC A61K 38/12; A61P 31/04; A61K 47/61

(57) ABSTRACT

The present disclosure provides a conjugate comprising an anti-microbial agent and a hydrophilic polymer; and compositions, including pharmaceutical compositions, comprising the conjugates. The present disclosure provides a conjugate comprising a polymyxin covalently linked to a maltodextrin polymer; and compositions, including pharmaceutical compositions, comprising the conjugates. The present disclosure provides methods of inhibiting growth of a bacterium, and methods of treating a bacterial infection.

### CROSS-REFERENCE

This application claims the benefit of U.S. Provisional Patent Application No. 62/486,818, filed Apr. 18, 2017, which application is incorporated herein by reference in its entirety.

### INTRODUCTION

The treatment of bacterial infections is a central challenge in medicine. For example, in the United States, in 2010, bacterial infections killed more people than AIDs, breast cancer and prostate cancer combined. Moreover, antibiotic resistance is a growing public health concern and there is an urgent need for effective treatments for drug resistant gram negative infections. According to Centers for Disease Control and Prevention (CDC), at least 2 million people in the US suffer from infection by drug resistant micro-organisms (bacteria and fungus combined), and 23,000 people die from it. Colistin is an effective antibiotic within the polymyxin family, and it is considered to be the “last resort drug” for drug resistant gram negative bacterial infections³. For example, colistin is effective against infections caused by multidrug resistant Pseudomonas aeruginosa, Klebsiella pneumoniae, and many other gram-negative bacteria. However, colistin causes severe nephrotoxicity (kidney toxicity), which limits its clinical use.

There is a need in the art for compositions and methods for inhibiting bacterial growth.

### SUMMARY

The present disclosure provides a conjugate comprising an anti-microbial agent and a hydrophilic polymer; and and compositions, including pharmaceutical compositions, comprising the conjugates. The present disclosure provides methods of inhibiting growth of a bacterium, the methods comprising contacting the bacterium with the conjugate. The present disclosure provides methods of treating a bacterial infection in an individual, the methods comprising administering to the individual an effective amount of the conjugate.

The present disclosure provides a conjugate comprising a polymyxin antibiotic and a maltodextrin polymer; and and compositions, including pharmaceutical compositions, comprising the conjugates. The present disclosure provides methods of inhibiting growth of a bacterium, the methods comprising contacting the bacterium with the conjugate. The present disclosure provides methods of treating a bacterial infection in an individual, the methods comprising administering to the individual an effective amount of the conjugate.

In some cases, a conjugate of the present discloses comprises: a) an antimicrobial agent; and b) a hydrophilic polymer, wherein the antimicrobial agent is covalently linked, directly or via a linker, to the hydrophilic polymer. In some cases, the conjugate exhibits reduced toxicity to an individual, compared to the toxicity exhibited by the antimicrobial agent in unconjugated form. In some cases, the side effects induced by the conjugate are reduced relative to the side effects induced by the antimicrobial agent in unconjugated form. “Toxicity to an individual” includes, e.g., nephrotoxicity, hepatotoxicity, neurotoxicity, ototoxicity, and the like.

In some cases, the antimicrobial agent of a conjugate of the present disclosure is a polymyxin antibiotic, an aminoglycoside antibiotic, a cationic antimicrobial peptide, or a dibasic macrolide antibiotic. In some cases, the polymyxin antibiotic is colistin, colistin sulfate, colistin methane-sulfonate, or a polymyxin derivative. In some cases, the antimicrobial agent is an antibody specific for a microbial antigen. In some cases, the antimicrobial agent is a polypeptide that enhances antimicrobial activity of an antibiotic. In some cases, the polypeptide that enhances antimicrobial activity of an antibiotic is polymyxin B nonapeptide, NAB7061, or NAB741.

In some cases, the polypeptide that enhances antimicrobial activity of an antibiotic is a polymyxin derivative of any one of Formulas I-LXXV.

In some cases, the antimicrobial agent is an agent that facilitates entry of an antibiotic into a microbial cell.

In some cases, the hydrophilic polymer is poly(ethylene glycol) (PEG), poly(ethylene oxide) (PEO), poly(N-isopropylacrylamide) (PNIPAM), poly(2-oxazoline), polyethylenimine (PEI), poly(vinyl alcohol) (PVA), or poly(vinylpyrrolidone) (PVP).

In some cases, the hydrophilic polymer is a maltodextrin polymer. In some cases, the maltodextrin polymer is maltotriose, maltotetraose, maltopentaose, maltohexaose, maltoheptaose, maltooctaose, maltononaose, or maltodecaose. In some cases, the maltodextrin polymer comprises from 2 to 20,000 α (1→4)-linked D-glucose subunits.

In some cases, the polymer has a molecular weight of from about 0.5 Da to about 2000 kDa.

In some cases, the antimicrobial agent is conjugated to the hydrophilic polymer via a cleavable linker. In some cases, the cleavable linker is a proteolytically cleavable linker. In some cases, the cleavable linker is a water-hydrolyzable linker.

In some cases, the antimicrobial agent is conjugated to the hydrophilic polymer via a cleavable linker. In some cases, wherein the cleavable linker is a self-immolative linker. In some cases, the self-immolative linker is cleavable by a thiol. In some cases, the thiol is glutathione. In some cases, the cleavable linker is a water-hydrolyzable linker.

In some cases, the molar ratio of antimicrobial agent to hydrophilic polymer is from 1:1 to 100:1.

The present disclosure provides a pharmaceutical composition comprising: the conjugate comprising: a) an antimicrobial agent; and b) a hydrophilic polymer, wherein the antimicrobial agent is covalently linked, directly or via a linker, to the hydrophilic polymer; and a pharmaceutically acceptable excipient.

In some cases, the pharmaceutical composition is a liquid composition. In some cases, the composition is an aerosol. In some cases, the composition a gel, a semi-solid, or a solid. In some cases, wherein the conjugate is present in the composition in a concentration of from 0.01 μg/ml to 200 mg/ml.

The present disclosure provides a method of inhibiting growth of a bacterium, the method comprising contacting the bacterium with the conjugate comprising: a) an antimicrobial agent; and b) a hydrophilic polymer, wherein the antimicrobial agent is covalently linked, directly or via a linker, to the hydrophilic polymer. In some cases, the bacterium is a gram-negative bacterium. In some cases, the bacterium is a gram-positive bacterium. In some cases, the bacterium is resistant to a carbapenem antibiotic. In some cases, the bacterium is resistant to more than one antibiotic. In some cases, the bacterium is Pseudomonas aeruginosa, Klebsiella pneumoniae, Acinetobacter baumannii, Escherichia coli, or Staphylococcus aureus.

In some cases, the minimum inhibitory concentration of the conjugate is from about 0.01 μg/ml to 10 μg/ml of unconjugated antimicrobial agent equivalents.

The present disclosure provides a method of treating a bacterial infection in an individual, the method comprising administering to the individual an effective amount of the conjugate comprising a) an antimicrobial agent; and b) a hydrophilic polymer, wherein the antimicrobial agent is covalently linked, directly or via a linker, to the hydrophilic polymer. In some cases, the the individual is a human. In some cases, the individual is a non-human animal. In some cases, the non-human animal is a mammal.

In some cases, the conjugate is administered in a dose of from about 1 mg/kg per day to about 100 mg/kg per day, wherein the dose is based on the amount of equivalents of unconjugated antimicrobial agent.

In some cases, the conjugate is administered via oral administration. In some cases, the conjugate is administered via pulmonary administration. In some cases, the conjugate is administered via inhalational administration. In some cases, the conjugate is administered via intranasal administration. In some cases, the conjugate is administered via mucosal administration. In some cases, the conjugate is administered via topical administration. In some cases, the conjugate is administered via ocular administration. In some cases, the conjugate is administered via intravenous administration. In some cases, the conjugate is administered via subcutaneous administration.

In some cases, the method of treating a bacterial infection in an individual further comprises administering at least one additional therapeutic agent. In some cases, the at least one additional therapeutic agent is an antibiotic that is different from the antimicrobial agent in the conjugate. In some cases, the antibiotic is rifampicin, rifabutin, rifalazil, rifapentine, rifaximin, oxacillin, methicillin, ampicillin, cloxacillin, carbenicillin, piperacillin, tricarcillin, flucloxacillin, nafcillin, azithromycin, clarithromycin, erythromycin, telithromycin, cethromycin, solithromycin, aztreonam, BAL30072, meropenem, doripenem, imipenem, ertapenem, biapenem, tomopenem, panipenem, tigecycline, omadacycline, eravacycline, doxycycline, minocycline, ciprofloxacin, levofloxacin, moxifloxacin, delafloxacin, fusidic acid, novobiocin, teichoplanin, telavancin, dalbavancin, or oritavancin, or a pharmaceutically acceptable salt or solvates of same.

In some cases, the the individual is a human. In some cases, the individual is a non-human animal. In some cases, the non-human animal is a mammal.

### BRIEF DESCRIPTION OF THE DRAWINGS

FIG. 1 shows that MDP-2 can image E. coli in vivo.

FIG. 2 shows the chemical structure of Colistin-Maltodextrin Conjugate (CMC).

FIG. 3 shows a schematic illustration of targeted antimicrobial effect of Colistin-Maltodextrin Conjugate.

FIG. 4 shows the synthetic route to CMC.

FIG. 5 shows an evaluation of the antimicrobial effect of CMC and its MICs.

FIG. 6 shows MICs of Colistin and CMC against different strains of bacteria.

FIG. 7 depicts MIC of CMC, or CMC+glutathione, against various bacterial strains. “ATCC” refers to E. coli ATCC 25922.

FIG. 8 depicts MIC of maltodextrin, maltodextrin-linker, and CMC without TCEP and spinfiltration, against E. coli ATCC 25922.

FIG. 9 depicts toxicity of CMC to mammalian cells. The units on the x-axis are μg/ml.

FIG. 10 depicts bio-distribution of colistin after injection of CMC into infected mice.

FIG. 11 depicts pharmacokinetics of colistin and CMC.

FIG. 12 depicts the effect of CMC on urinary tract infection.

FIG. 13 depicts the effect of CMC on urinary tract infection.

FIG. 14 depicts the effect of free colisin or colistin-maltodextrin on bacterial counts in the bladder. CFU: colony-forming units.

### DEFINITIONS

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. A treatment method of the present disclosure will desirably be administered during the symptomatic stage of the disease, and in some cases after the symptomatic stage of the disease.

The terms “individual,” “subject,” “host,” and “patient,” are used interchangeably herein and refer to any mammalian subject for whom treatment or therapy is desired. Mammals include, e.g., humans, non-human primates, rodents (e.g., rats; mice), lagomorphs (e.g., rabbits), ungulates (e.g., cows, sheep, pigs, horses, goats, camels, and the like), felines (e.g., cats), canines (e.g., dogs), etc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a hydrophilic polymer” includes a plurality of such polymers and reference to “the anti-microbial agent” includes reference to one or more anti-microbial agents and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a conjugate comprising a polymyxin covalently linked to a maltodextrin polymer; and compositions, including pharmaceutical compositions, comprising the conjugates. The present disclosure provides methods of inhibiting growth of a bacterium, and methods of treating a bacterial infection.

Antimicrobial Agent-Hydrophilic Polymer Conjugates

The present disclosure provides a conjugate comprising an antimicrobial agent covalently linked to a hydrophilic polymer.

In some cases, the conjugate exhibits reduced toxicity to an individual compared to the toxicity exhibited by the antimicrobial agent in unconjugated forom. In some cases, the side effects induced by the conjugate are reduced relative to the side effects induced by the antimicrobial agent in unconjugated form.

In some case, the antimicrobial agent is a polymyxin antibiotic, an aminoglycoside antibiotic, a cationic antimicrobial peptide, or a dibasic macrolide antibiotic. The conjugate of claim 1, wherein the antimicrobial agent is a polypeptide that enhances antimicrobial activity of an antibiotic.

Polymyxins

In some cases, the antimicrobial agent is a polymyxin antibiotic. In some cases, the polymyxin antibiotic is colisting, colistin sulfate, colistin methane-sulfonate, or a polymyxin derivative.

In some cases, the antimicrobial agent is an antibody specific for a microbial antigen. In some cases, the antimicrobial agent is a polypeptide that enhances antimicrobial activity of an antibiotic.

Polymyxins suitable for inclusion in a conjugate of the present disclosure include polymyxin B and polymyxin E (also known as colistin); a polymyxin derivative disclosed in WO 2008/017734, where the polymyxin derivative carries at least two but no more than three positive charges; a des-fatty acyl polymyxin derivative (see, e.g., Katsuma et al. (2009) Chem. Pharm. Bull. 57:332; and Sato et al. (2011) Chem. Pharm. Bull. 59:597); a urea-linked aryl polymyxin decapeptide as described in WO 2010/075416, e.g., CB102,804 (see also Quale et al. (2012) Microb. Drug Resist. 18:132-136; a phenyl cyclopropane polymyxin derivative as described in U.S. Pat. No. 8,415,307; a polymyxin derivative, as described in WO 2012/168820, in which the diaminobutyrate group at position 3 in the tripeptide side chain is replaced by a diaminopropionate moiety; a polymyxin derivative, as described in WO 2009/098357, in which the terminal moiety (D) of the derivative comprises a total of 1 to 5 carbon atoms.

Polymyxins are a group of closely related antibiotic substances produced by strains of Paenibacillus polymyxa and related organisms. These cationic drugs are relatively simple peptides with molecular weights of about 1000. Polymyxins, such as polymyxin B, are decapeptide antibiotics, i.e., they are made of ten (10) aminoacyl residues. They are bactericidal and especially effective against Gram-negative bacteria such as Escherichia coli and other species of Enterobacteriaceae, Pseudomonas, Acinetobacter baumannii, and others. However, polymyxins have severe adverse effects, including nephrotoxicity and neurotoxicity. These drugs thus have limited use as therapeutic agents because of high systemic toxicity. Structural formulas of Polymyxins and polymyxin derivatives are well-known in the art and can be found in, for example, U.S. Patent Application Publication No.: 2014/0162937, and 2013/0345121 which are hereby incorporated by reference in their entirety.

Polymyxins consist of a cyclic heptapeptide part and a linear part consisting of a tripeptide portion and a hydrophobic fatty acid tail linked to the α-amino group of the N-terminal amino acid residue of the tripeptide and may be represented by the general formula:

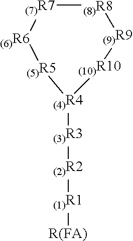

wherein R1-R3 represent the tripeptide side chain portion; R4-R10 the heptapeptide ring portion and R(FA) represents the hydrophobic fatty acid tail linked to the α-amino group of the N-terminal amino acid residue of the tripeptide.

The polymyxin group includes the following polymyxins: A1, A2, B1-B6, IL-polymyxin B1, C, D1, D2, E1, E2, F, K1, K2, M, P1, P2, S, and T (Storm et al. 1977; Srinivasa and Ramachandran 1979). All polymyxins are polycationic and possess five (5) positive charges, with the exception of polymyxin D, F, and S which possess four (4) positive charges. It should be noted that modified polymyxins that lack the fatty acid part R(FA) but carry R1-R10 have one additional positive charge when compared to the natural polymyxins they derived from, due to the free α-amino group in the N-terminus of the derivative. Accordingly, for example, such a derivative of polymyxin B or polymyxin E carries six (6) positive charges in total. Also, circulin A and B are classified as polymyxins (Storm et al. 1977). They differ from other polymyxins only in carrying isoleucyl residue in the position R7 whereas other polymyxins have either threonyl or leucyl residue in the said position.

The present disclosure provides herein a conjugate comprising an antimicrobial agent and a hydrophilic polymer, wherein the antimicrobial agent is covalently linked, directly or via a linker, to the hydrophilic polymer.

Suitable antimicrobial agents include polymyxin derivatives disclosed in U.S. Patent Application Publication No.: 2014/0162937, 2013/0345121, and International Patent Application Nos. WO 2012/168820 and WO 2009/098357. The disclosures of all the foregoing references are incorporated herein by reference in their entirety.

In some cases, a suitable polymyxin derivative is a compound of Formula (I):

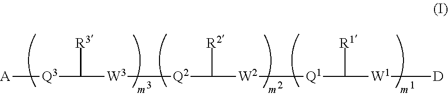

wherein:

A is a polymyxin ring moiety;

D is a terminal moiety;

m¹, m², and m³ are each independently 0 or 1;

Q¹, Q² and Q³ are each independently CH₂, C═O, or C═S;

W¹, W², and W³ are each independently NR⁴, O, or S;

R^{1′}, R^{2′}, and R^{3′} are each independently side chains of natural or unnatural amino acids, alkyl, alkenyl, arylalkyl, aryl, alkoxy, alkoxycarbonyl, aryloxycarbonyl, alkylamino, or alkynyl; and

R⁴ is hydrogen or alkyl,

and pharmaceutically acceptable prodrugs and salts thereof, wherein said derivative has at least two but no more than three positive charges at physiological pH, provided that when (1) A is a polymyxin B ring moiety, and m¹, m², and m³ are each 0, then D is not octanoyl; (2) when D is (S)-6-methyloctanoyl, hexanoyl, myristoyl, octanoyl or octanoyl-Dab, then R³ is not the side chain of Dab; or (3) provided that said derivative is not 8,9-diformylated polymyxin B.

Examples of prodrugs of these derivatives include those with charge masking moieties which neutralize the positive charges when administered to the subject which are removed in vivo to yield the compound with three positive charges. Examples of charge masking moieties include sulfoalkyl moieties such as sulfomethyl.

In some cases, the derivatives have three positive charges at physiological pH, as defined above. In certain embodiments of the invention, R^{1′}, R^{2′}, and R^{3′} do not comprise positively charged functional groups at physiological pH. R^{1′}, R^{2′}, and R^{3′} may comprise, for example, one or two or more carbamyl, hydroxyl, carboxylate, thiol, sulfate, sulfonyl, or phosphate groups. However, in certain embodiments of the invention, R¹, R^{2′} and R^{3′} may comprise one or more positively charged functional groups.

In some cases, m¹ is 0 and m² and m³ are each 1. In another, Q² and Q³ are each C═O and W² and W³ are each NH.

In some cases, R^{2′} is substituted with one or more groups selected from carbamyl, hydroxyl, carboxylate, thiol, sulfate, sulfonyl, or phosphate groups. In some cases, R^{2′} is substituted with a carbamyl, hydroxyl or carboxylate group. Examples of R^{2′} include alanine, aminobutyric acid, asparagine, aspartic acid, diaminobutyric acid, glutamic acid, glutamine, serine, and threonine, in either L or D configuration. In some cases, R^{2′} is the side chain of D-alanine, L-serine, or L-threonine.

In some cases, R^{3′} is substituted with one or more groups selected from carbamyl, hydroxyl, carboxylate, thiol, sulfate, sulfonyl, or phosphate. In some cases, R^{3′} is substituted alkyl and maybe substituted with a carbamyl, hydroxyl or carboxylate group. Examples of R^{3′} include alanine, aminobutyric acid, asparagine, aspartic acid, diaminobutyric acid, glutamic acid, glutamine, serine, and threonine, in either L or D configuration. In some cases, R^{3′} is D-alanine, L-aminobutyric acid, L-asparagine, D-asparagine, L-diaminobutyric acid, L-serine, D-serine, or D-threonine.

Examples of A include the ring moiety of polymyxin B (i.e., cy[Dab-Dab-DPhe-Leu-Dab-Dab-Thr]) and polymyxin E (i.e., cy[Dab-Dab-DLeu-Leu-Dab-Dab-Thr-].

In some cases, the terminal moiety is selected from the group consisting of a hydrophobic oligopeptide, R¹²—(C═O); R¹²—SO₂—; R¹²—(C═NH)—; R¹²—NH—(C═S)—; R¹²—NH—(CO)—; R¹²—NH—(C═NH)—; R¹²—O—(C═S)—; R¹²—O—(C═O); R¹²-p(O)OH—; R¹² (C═S); and R¹², wherein R¹² is alkyl, cycloalkyl, alkenyl, alkynyl, aryl, or aryl alkyl. In certain embodiments, D is R¹²—(C═O) or R¹²—(C═S).

Examples of hydrophobic oligopeptides that can be used in the derivatives of the invention include oligopeptides of 1-10, or, in some cases, 2-5, amino acyl residues (e.g., Leu, lie, Phe, or Trp), such as, but not limited to Leu-Leu-Leu, IIe-Leu-IIe, Phe-IIe-Leu and Trp-Trp-IIe.

Examples of D include octanoyl, nonanoyl, isononanoyl, decanoyl, isodecanoyl, undecanoyl, dodecanoyl, tetradecanoyl, cyclohexyl, cycloheptanoyl, cyclooctanoyl, cyclononanoyl, cycloisononanoyl, cyclodecanoyl, cycloisodecanoyl, cycloundecanoyl, cyclododecanoyl, cyclotetradecanoyl, hexanoyl, heptanoyl, and 9-fluorenylmethoxycarbonyl. In a further embodiment, D contains 6 to 18 carbon atoms. In a further embodiment, D is 6-methyloctanoyl; 6-methylheptanoyl; 3-OH-8-methyldecanoyl; or octanoyl.

In some cases, A is a polymyxin ring moiety selected from that of polymyxin A, polymyxin B, IL-polymyxin-B1, polymyxin D, polymyxin E, polymyxin F, polymyxin M, polymyxin S, polymyxin T, circulin A, octapeptin A, octapeptin B, octapeptin C, octapeptin D, or derivatives thereof. In some cases, A is a polymyxin ring moiety of polymyxin B or polymyxin E.

In some cases, a suitable polymyxin derivative is a compound of Formula (II):

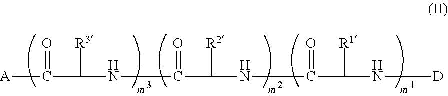

wherein:

A is a polymyxin ring moiety;

D is a hydrophobic oligopeptide, R¹²—C(═O) or R¹²—C(═S);

m¹, m², and m³ are each independently 0 or 1;

R¹, R^{2′}, and R^{3′} are each independently side chains of natural or unnatural amino acids, alkyl, alkenyl, alkyl, arylalkyl, aryl, alkoxy, alkoxycarbonyl, aryloxycarbonyl, alkylamino, or alkynyl; and

R¹² is C₅-C₁₇ alkyl, C₅-C₁₇ alkenyl, C₅-C₁₇ aryl, C₅-C₁₇ arylalkyl or C₅-C₁₇ alkynyl,

and pharmaceutically acceptable prodrugs and salts thereof, wherein said derivative has at least two and no more than three positive charges at physiological pH, and provided (1) when A is a polymyxin B ring moiety, and m¹, m², and m³ are each 0, then D is not octanoyl; (2) when D is (S)-6-methyloctanoyl, hexanoyl, myristoyl, octanoyl or octanoyl-Dab, then R³ is not the side chain of Dab; or (3) provided that said derivative is not 8,9-diformylated polymyxin B.

In some cases, the derivative of formula (II) has three positive charges at physiological pH. In a further embodiment, m¹ may be 0 and/or m² and m³ may each be 1. In a further embodiment, R^{2′} and/or R^{3′} may each independently be substituted alkyl (e.g., substituted with a carbamyl, hydroxyl or carboxylate group). Furthermore, R^{2′} and/or R³ may each be the side chain of alanine, aminobutyric acid, asparagine, aspartic acid, diaminobutyric acid, glutamic acid, glutamine, serine, and threonine, in either the L or D configuration. In some cases, R^{2′} is the side chain of D-alanine, L-serine, or L-threonine and R^{3′} is D-alanine, L-aminobutyric acid, L-asparagine, D-asparagine, L-diaminobutyric acid, L-serine, D-serine, or D-threonine.

In some cases, R¹² is alkyl. Examples of D include octanoyl, nonanoyl, isononanoyl, decanoyl, isodecanoyl, undecanoyl, dodecanoyl, tetradecanoyl, cyclohexyl, cycloheptanoyl, cyclooctanoyl, cyclononanoyl, cycloisononanoyl, cyclodecanoyl, cycloisodecanoyl, cycloundecanoyl, cyclododecanoyl, cyclotetradecanoyl, hexanoyl, heptanoyl, and 9-fluorenylmethoxycarbonyl.

In some cases, a suitable polymyxin derivative is a compound of Formula (III):

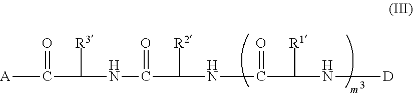

wherein:

A is a polymyxin B or polymyxin E ring moiety;

D is R¹²—C(═O) or R¹²—C(═S);

m¹ is 0 or 1;

R^{1′}, R^{2′}, and R^{3′} are each independently side chains of natural or unnatural amino acids, alkyl, alkenyl, arylalkyl, aryl, alkoxy, alkoxycarbonyl, aryloxycarbonyl, alkylamino, or alkynyl, wherein at least one of R^{2′} and R^{3′} comprise a carbamyl, hydroxyl or carboxylate group; and

R¹² is C₅-C₁₇ alkyl,

and pharmaceutically acceptable prodrugs and salts thereof, wherein said derivative has at least two but no more than three positive charges at physiological pH, and provided (1) when D is (S)-6-methyloctanoyl, hexanoyl, myristoyl, octanoyl or octanoyl-Dab, then R³ is not the side chain of Dab; or (2) said derivative is not 8,9-diformylated polymyxin B.

In some cases, the compounds of the invention have three positive charges at physiological pH, m¹ is 0, R^{2′} and R³ are both substituted alkyl, and/or D is octanoyl, nonanoyl, isononanoyl, decanoyl, isodecanoyl, undecanoyl, dodecanoyl, tetradecanoyl, cyclohexyl, cycloheptanoyl, cyclooctanoyl, cyclononanoyl, cycloisononanoyl, cyclodecanoyl, cycloisodecanoyl, cycloundecanoyl, cyclododecanoyl, cyclotetradecanoyl, hexanoyl or heptanoyl.

In some cases, a suitable polymyxin derivative is a compound of Formula (IV):

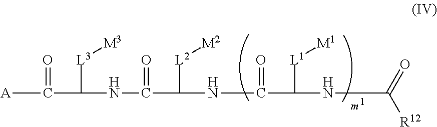

wherein:

A is a polymyxin B or polymyxin E ring moiety;

m′ is 0 or 1;

L¹, L² and L³ are each independently C₁-C₃ alkyl or a covalent bond;

M¹, M² and M³ are each independently H, NH₂, C(═O)NH₂, C(═O)OH, or —OH;

R¹² is C₅-C₁₇ alkyl,

and pharmaceutically acceptable prodrugs and salts thereof, and wherein said derivative has at least two but no more than three positive charges at physiological pH, and provided (1) when D is (S)-6-methyloctanoyl, hexanoyl, myristoyl, octanoyl or octanoyl-Dab, then R³ is not the side chain of Dab; or (2) said derivative is not 8,9-diformylated polymyxin B.

Examples of L² include branched alkyl (e.g., —CH(CH₃)—) and methylene (—CH₂). Examples of M² include OH and H. In another embodiment, L³ is —CH₂— and M³ is OH or H. In yet another embodiment, L³ is —CH₂—CH₂— and M³ is C(═O)NH₂. Other examples of L³ include —CH(CH₃)— and CH(CH₂CH₃)— wherein M³ is OH or NH₂.

In some cases, a suitable polymyxin derivative is a compound of Formula (V):

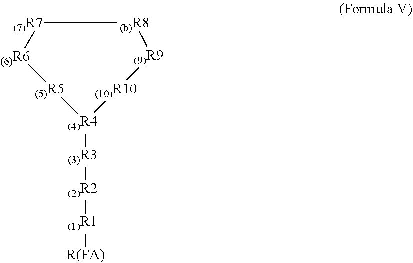

wherein R4 is an amino acid residue comprising a functional side chain able to cyclicize the molecule;

R6 is an optionally substituted hydrophobic amino acid residue;

R7 is an optionally substituted hydrophobic residue;

R10 is Leu or any non-hydrophobic amino acid residue; and

wherein R1, R2, and R3 are optional; and wherein R1, R2, R3, R5, R8 and R9 are amino acid residues selected so that the total number of positive charges at physiological pH is at least two and no more than three; and

wherein R(FA) is an optionally substituted alkanoyl or alkyl residue or a hydrophobic oligopeptide;

or a pharmaceutically acceptable prodrug or salt thereof, provided that (1) when R8 and R9 are formylated, R(FA)-R1-R2-R3 does not constitute a native polymyxin B side chain; (2) when R4-R10 is a polymyxin B ring moiety and R1, R2, and R3 are each absent, then R(FA) is not octanoyl, or (3) when R(FA) is (S)-6-methyloctanoyl, hexanoyl, myristoyl, octanoyl or octanoyl-Dab, then R3 is not the side chain of Dab.

In natural polymyxins and octapeptins, R(FA) is 6-methyloctanoic acid (6-MOA), 6-methylheptanoic acid (6-MHA), octanoic acid, heptanoic acid, nonanoic acid, 3-OH-6-methyloctanoic acid, 3-OH-8-methyldecanoic acid, 3-OH-8-methylnonanoic acid, 3-OH-8-decanoic acid, and 3-OH-6-methyloctanoic acid. Examples of known derivatives that have antibacterial activity include those wherein R(FA) is γ-phenylbutyric acid, isovaleric acid, 9-fluorenyl-methoxycarbonic acid, a series of C:9 to C:14 unbranched fatty acids as well as iso C:9 and iso C:10 fatty acids.

In a derivative according to the present invention, R(FA) may be any hydrophobic fatty acid residue, or may be selected from the group consisting of octanoyl, decanoyl and 6-MHA residues.

A person skilled in the art may readily recognize equivalents of these hydrophobic R(FA) residues, which may be selected from the group consisting of e.g. optionally substituted acyl or alkyl residue, an optionally substituted isoalkyl residue, an optionally substituted cycloalkyl residue, an optionally substituted alkenyl residue, an optionally substituted cycloalkenyl residue, an optionally substituted aryl residue, an optionally substituted heteroaryl residue, an optionally substituted heterocyclic residue, wherein said residues, In some cases, have more than five (5) carbon atoms and wherein the substitutions may also include those optionally designed between the residue and the N-terminus of the peptide. R(FA) may also be a stretch of a hydrophobic oligopeptide. Examples of possible R(FA) residues include (but are not limited to) octanoyl, nonanoyl, isononanoyl, decanoyl, isodecanoyl, undecanoyl, dodecanoyl, tetradecanoyl, cyclohexanoyl, cycloheptanoyl, cyclooctanoyl, cyclononanoyl, cycloisononanoyl, cyclodecanoyl, cycloisodecanoyl, cycloundecanoyl, cyclododecanoyl, cyclotetradecanoyl, hexanoyl, heptanoyl, and 9-fluorenylmethoxycarbonyl residues.

In natural polymyxins and octapeptins, R1 is Dab or absent (i.e., replaced by a covalent bond). Examples of known derivatives that have antibacterial activity include those wherein R1 is Ala or a covalent bond.

In a derivative according to the present invention R1, if present, may be any amino acid residue, provided that the total number of positive charges in said derivative does not exceed three and that the total number of positive charges in the side chain portion does not exceed two, and is In some cases, Abu, if present.

In natural polymyxins and octapeptins, R2 is Thr or absent (i.e., replaced by a covalent bond). Examples of known derivatives that have antibacterial activity include those wherein R2 is O-acetyl-Thr, O-propionyl-Thr, O-butyryl-Thr or a covalent bond.

In a derivative according to the present invention, R2, if present, may be any amino acid residue, provided that the total number of positive charges in said derivative does not exceed three and that the total number of positive charges in the side chain portion does not exceed two, and is in some cases, selected from the group consisting of alanine, aminobutyric acid, asparagine, aspartic acid, diaminobutyric acid, glutamic acid, glutamine, serine, and threonine, in either L or D configuration, if present. A person skilled in the art may also recognize an equivalent residue of Thr to be Ser.

In natural polymyxins and octapeptins, R3 is Dab, DDab or DSer. Examples of numerous known synthetic derivatives that have antibacterial activity include those wherein R3 is Lys or 2-amino-4-guanidino butyric acid.

In a derivative according to the present invention, R3, if present, may be any amino acid residue, provided that the total number of positive charges in said derivative does not exceed three and that the total number of positive charges in the chain portion does not exceed two, and is in some cases, selected from the group consisting of alanine, aminobutyric acid, asparagine, aspartic acid, diaminobutyric acid, glutamic acid, glutamine, serine, and threonine, in either L or D configuration, if present.

A person skilled in the art may readily recognize residues other than these residues R1, R2 and R3, and may select such from a group consisting of e.g. a covalent bond, alanine, 2-aminoadipic acid, α-n-butyric acid, N-(4-aminobutyl)glycine, α-aminobutyric acid, γ-aminobutyric acid, α-amino-caproic acid, aminocyclopropanecarboxylate, aminoisobutyric acid, aminonorbornylcarboxylate, α-amino-n-valeric acid, arginine, N,-methyl arginine, asparagine, α-methylaspartate, aspartic acid, N-benzylglycine, N-(2-carbamylethyl)glycine, N-(carbamylethyl)glycine, 1-carboxy-1(2,2-diphenyl ethylamino)cyclopropane, cysteine, Na-methyldiamino-n-butyric acid, N_{γ}-acetyldiamino-n-butyric acid, N_{γ}-formyldiamino-n-butyric acid, N_{γ}-methyl-diamino-n-butyric acid, N—(N-2,2-diphenylethyl)carbamylmethyl-glycine, N—(N-3,3-diphenylpropyl) carbamylmethyl(1)glycine, N-(3,3-diphenylpropyl)glycine, glutamic acid, glutamine, glycine, t-butylglycine, 2-amino-4-guanidinobutyric acid, N-(3-guanidinopropyl)glycine, histidine, homophenylalanine, isodesmosine, isoleucine, leucine, norleucine, hydroxylysine, N_{α}-methyllysine, lysine, N_{α}-methylhydroxylysine, N_{α}-methyllysine, N_{ε}-acetylhydroxylysine, N_{ε}-acetyl lysine, N_{ε}-formylhydroxylysine, N_{ε}-formyllysine, N_{ε}-methylhydroxylysine, N_{ε}-methyllysine, methionine, α-methyl-γ-aminobutyrate, α-methyl-aminoiso butyrate, α-methylcyclohexylalanine, α-napthylalanine, norleucine, norvaline, α-methylornithine, N_{α}-methylornithine, N_{δ}-acetylornithine, N_{δ}-formyl-ornithine, N_{δ}-methylornithine, ornithine, penicilamine, phenylalanine, hydroxyproline, proline, N_{α}-methyldiamino-n-propionic acid, N_{β}-acetyldiamino-n-propionic acid, N_{β}-formyldiamino-n-propionic acid, N_{β}-methyldiamino-n-propionic acid, phosphoserine, serine, phosphothreonine, threonine, tryptophan, tyrosine, norvaline, and valine.

In natural polymyxins and octapeptins, R4 is Dab. Examples of synthetic derivatives that have antibacterial activity include those wherein R4 is Lys.

In a derivative according to the present invention R4 is an amino acid residue comprising a functional side chain able to cyclicize the molecule, and may be selected from the group of equivalent residues consisting of Lys, hydroxylysine, ornithine, Glu, Asp, Dab, diaminopropionic acid, Thr, Ser and Cys, and in some cases, Dab.

In natural polymyxins and octapeptins, R5, R8 and R9 are Dab. Examples of synthetic derivatives that have antibacterial activity include those wherein R5, R8, and R9 may be Lys or 2-amino-4-guanidino butyric acid.

In a derivative according to the present invention R5, R8 and R9 may be a positively charged or a neutral amino acid residue, in some cases, Dab or Abu, provided that the total number of positive charges in said derivative does not exceed three.

A person skilled in the art, may readily recognize equivalent residues of these residues, and may select such from a group consisting of e.g. diaminobutyric acid, diaminopropionic acid, lysine, hydroxylysine, ornithine, 2-amino-4-guanidinobutyric acid, glycine, alanine, valine, leucine, isoleucine, phenylalanine, D-phenylalanine, methionine, threonine, serine, α-amino-n-butyric acid, α-amino-n-valeric acid, α-amino-caproic acid, N_{ε}-formyl-lysine, N_{ε}-acetyllysine, N_{ε}-methyllysine, N_{ε}-formylhydroxylysine, N_{ε}-acetylhydroxylysine, N_{ε}-methylhydroxylysine, L-N_{α}-methylhydroxylysine, N_{γ}-formyldiamino-n-butyric acid, N_{γ}-acetyldiamino-n-butyric acid, N-methyldiamino-n-butyric acid, N_{β}-formyldiamino-n-propionic acid, D-N_{β}-formyldiamino-n-propionic acid, N_{β}-acetyldiamino-n-propionic acid, N_{β}-methyldiamino-n-propionic acid, N_{α}-formylornithine, N_{δ}-acetylornithine and N_{δ}-methylornithine.

In natural polymyxins and octapeptins, R6 is DPhe or DLeu and R7 is Leu, Ile, Phe or Thr. Synthetic derivatives that have antibacterial activity include those wherein R6 is DTrp and wherein R7 is Ala. In a derivative according to the present invention, R6 is an optionally substituted hydrophobic amino acid residue, in some cases, DPhe or DLeu, and R7 is an optionally substituted hydrophobic residue, in some cases, Leu, Thr or IIe.

A person skilled in the art may readily recognize equivalent residues of these hydrophobic residues, and may select such from a group consisting of e.g. phenylalanine, α-amino-n-butyric acid, tryptophane, leucine, methionine, valine, norvaline, norleucine, isoleucine and tyrosine. A person skilled in the art may also recognize the equivalent residue of threonine to be serine.

In natural polymyxins and octapeptins, R10 is Thr and Leu. Examples of known derivatives that have antibacterial activity include those wherein R10 is O-acetyl-Thr, O-propionyl-Thr or O-butyryl-Thr.

In some cases, R10 is Leu or any non-hydrophobic amino acid residue, provided that that the total number of positive charges in said derivative does not exceed three. In some cases, R10 is Thr or Leu. In some cases, serine is substituted for threonine.

More specifically, in some cases, residues are chosen in such a manner that R8 and R9 are not both formylated when R(FA)-R1-R2-R3 constitutes the native polymyxin B sidechain; and R4 is not directly linked to octanoyl residue when R4-R10 constitutes a native polymyxin B ring structure.

The specific positions of the at the most three (3) positive charges referred to herein above can be located in the heptapeptide ring portion and/or in the side chain, if present. When three (3) positive charges are present in the derivatives according to the invention, said three (3) positive charges can be located in the heptapeptide ring portion; or two (2) positive charges can be located in heptapeptide ring portion while the remaining one positive charge is located in the side chain; or one (1) positive charge can be located in the heptapeptide ring portion while the remaining two (2) positive charges are located in the side chain. In some cases, at least two (2) positive charges are located in the heptapeptide ring portion.

In some cases, a derivative can be selected from the group of derivatives wherein R1-R10 is selected from the group consisting of Thr-DSer-cy[Dab-Dab-DPhe-Leu-Dab-Dab-Thr-]; Thr-DThr-cy[Dab-Dab-DPhe-Thr-Dab-Dab-Thr-]; Thr-DSer-cy[Dab-Dab-DPhe-Thr-Dab-Dab-Thr-]; Thr-Abu-cy[Dab-Dab-DPhe-Leu-Dab-Dab-Thr-]; Abu-Thr-Abu-cy[Dab-Dab-DPhe-Leu-Dab-Dab-Thr-]; Thr-Dab-cy[Dab-Dab-DPhe-Leu-Abu-Dab-Thr-]; Thr-Abu-cy[Dab-Dab-DPhe-Leu-Dab-Dab-Leu-]; Thr-OAla-cy[Dab-Dab-DPhe-Thr-Dab-Dab-Thr-]; Thr-Dab-cy[Dab-Dab-DPhe-Leu-Dab-Abu-Thr-]; Thr-Abu-cy[Dab-Dab-OLeu-Leu-Dab-Dab-Thr-]; OAla-OAla-cy[Dab-Dab-OPhe-Leu-Dab-Dab-Thr-]; cy[Dab-Dab-DPhe-Leu-Dab-Dab-Thr-], Abu-cy[Dab-Oab-DPhe-Leu-Dab-Dab-Thr-]; Thr-Dab-cy[Dab-Abu-DPhe-Leu-Dab-Dab-Thr-]; Dab-Thr-Dab-cy[Dab-Dab-DPhe-Leu-Abu-Abu-Thr-]; Thr-Abu-cy[Dab-Lys-DPhe-Leu-Dab-Dab-Thr-]; Thr-Abu-cy[Dab-Abu-DPhe-Leu-Dab-Dab-Thr-]; Thr-Abu-cy[Dab-Dab-DPhe-Leu-Dab-Abu-Thr-]; Thr-Ser-cy[Dab-Dab-DPhe-Leu-Dab-Dab-Thr-]; Thr-Asn-cy[Dab-Dab-DPhe-Leu-Dab-Dab-Thr-]; Thr-Thr-OSer-cy[Dab-Dab-DPhe-Leu-Dab-Dab-Thr-]; and Ala-Thr-DSer-cy[Dab-Dab-DPhe-Leu-Dab-Dab-Thr-].

In some cases, a derivative can be selected from the group consisting of: OA-Thr-DSer-cy[Dab-Dab-OPhe-Leu-Dab-Dab-Thr-]; DA-Thr-DSer-cy[Dab-Dab-DPhe-Leu-Dab-Dab-Thr-]; DA-Thr-OThr-cy[Dab-Dab-DPhe-Thr-Dab-Dab-Thr-]; OA-Thr-DSer-cy[Dab-Oab-DPhe-Thr-Dab-Dab-Thr-]; DA-Thr-Abu-cy[Dab-Dab-DPhe-Leu-Dab-Dab-Thr-]; DA-Thr-Abu-cy[Dab-Dab-DPhe-Leu-Dab-Dab-Thr-]; MHA-Thr-Abu-cy[Dab-Dab-DPhe-Leu-Dab-Dab-Thr-]; MHA-Abu-Thr-Abu-cy[Dab-Dab-DPhe-Leu-Dab-Dab-Thr-]; OA-Thr-Dab-cy[Dab-Dab-DPhe-Leu-Abu-Dab-Thr-]; OA-Thr-Abu-cy[Dab-Dab-DPhe-Leu-Dab-Dab-Leu-]; OA-Thr-OAla-cy[Dab-Dab-OPhe-Thr-Dab-Dab-Thr-]; OA-Thr-Dab-cy[Dab-Dab-DPhe-Leu-Dab-Abu-Thr-]; OA-Thr-Abu-cy[Dab-Dab-OLeu-Leu-Dab-Dab-Thr-]; OA-OAla-OAla-cy[Dab-Oab-DPhe-Leu-Dab-Dab-Thr-]; OA-cy[Dab-Dab-DPhe-Leu-Dab-Dab-Thr-]; OA-Abu-cy[Dab-Dab-DPhe-Leu-Dab-Dab-Thr-]; OA-Thr-Dab-cy[Dab-Abu-DPhe-Leu-Dab-Dab-Thr-]; MHA-Dab-Thr-Dab-cy[Dab-Dab-DPhe-Leu-Abu-Abu-Thr-]; OA-Thr-Abu-cy[Dab-Lys-O Phe-Leu-Dab-Dab-Thr-]; OA-Thr-Abu-cy[Dab-Abu-DPhe-Leu-Dab-Dab-Thr-]; OA-Thr-Abu-cy[Dab-Dab-DPhe-Leu-Dab-Abu-Thr-]; OA-Thr-Ser-cy[Dab-Dab-DPhe-Leu-Dab-Dab-Thr-]; OA-Thr-Asn-cy[Dab-Oab-DPhe-Leu-Dab-Dab-Thr-]; OA-Thr-Thr-DSer-cy[Dab-Dab-DPhe-Leu-Dab-Dab-Thr-]; and OA-Ala-Thr-DSer-cy[Dab-Dab-DPhe-Leu-Dab-Dab-Thr-].

In some cases, a derivative is selected from the group consisting of: OA-Thr-DSer-cy[Dab-Dab-Dphe-Leu-Dab-Dab-Thr-]; OA-Thr-DSer-cy[Dab-Dab-DPhe-Leu-Dab-Dab-Thr-]; OA-Thr-Othr-cy[Dab-Dab-DPhe-Thr-Dab-Dab-Thr-]; OA-Thr-DSer-cy[Dab-Dab-DPhe-Thr-Dab-Dab-Thr-]; OA-Thr-Abu-cy[Dab-Dab-DPhe-Leu-Dab-Dab-Thr-]; OA-Thr-Abu-cy[Dab-Dab-DPhe-Leu-Dab-Dab-Thr-]; MHA-Thr-Abu-cy[Dab-Oab-Dphe-Leu-Dab-Dab-Thr-]; MHA-Abu-Thr-Abu-cy[Dab-Dab-O Phe-Leu-Oab-Dab-Thr-]; OA-Thr-Dab-cy[Dab-Dab-DPhe-Leu-Abu-Dab-Thr-]; OA-Thr-Abu-cy[Dab-Dab-DPhe-Leu-Dab-Dab-Leu-]; OA-Thr-OAla-cy[Dab-Dab-O Phe-Thr-Dab-Dab-Thr-]; OA-Thr-Dab-cy[Dab-Dab-DPhe-Leu-Dab-Abu-Thr-]; OA-Thr-Abu-cy[Dab-Dab-OLeu-Leu-Dab-Dab-Thr-]; OA-OAla-OAla-cy[Dab-Dab-Ophe-Leu-Dab-Dab-Thr-]; OA-cy[Dab-Dab-DPhe-Leu-Dab-Dab-Thr-]; OA-Thr-Ser-cy[Dab-Dab-DPhe-Leu-Dab-Dab-Thr-]; OA-Thr-Asn-cy[Dab-Dab-DPhe-Leu-Dab-Dab-Thr-]; OA-Thr-Thr-DSer-cy[Dab-Dab-DPhe-Leu-Dab-Dab-Thr-]; and OA-Ala-Thr-DSer-cy[Dab-Dab-DPhe-Leu-Dab-Dab-Thr-].

### Potentiators

The present application discloses a conjugate comprising an antimicrobial agent; and a hydrophilic polymer, wherein the antimicrobial agent is covalently linked, directly or via a linker, to the hydrophilic polymer. In some cases, the antimicbrial agent is a polypeptide that enhances antimicrobial activity of an antibiotic.

In some cases, the antimicrobial agent is an agent that facilitates entry of an antibiotic into a microbial cell. In some cases, the antimicrobial agent includes a cell penetrating peptide to facilitate entry of the antimicrobial agent across a cell membrane (see, e.g., US 2016/0289272). A variety of cell penetrating peptides (CPP) are known and described in the art, for example, in U.S. Patent Application Publication No.: 2016/028972, which is hereby incorporated by reference in its entirety. Cell penetrating peptides may vary greatly in size, sequence and charge, and in their mechanism of function, but share the common ability to translocate across the plasma membrane and deliver an attached or associated moiety (e.g. “cargo”) into the cytoplasm of a cell. CPPs are thus peptide-based delivery vectors.

CPPs are not characterized by a single structural or functional motif; however, tools to identify CPPs are available and the skilled person can readily determine whether a peptide sequence may function to facilitate the uptake of the peptide of which it forms a domain, i.e. whether a peptide sequence may function as an uptake (import) peptide, e.g. a CPP. For example, Hansen et al (Predicting cell-penetrating peptides, Advanced Drug Delivery Reviews, 2008, 60, pp. 572-579), provides a review of methods for CPP prediction based on the use of principal component analysis (“z-predictors”) and corresponding algorithms based on original work by Hallbrink et al (Prediction of Cell-Penetrating Peptides, International Journal of Peptide Research and Therapeutics, 2005, 11(4), pp. 249-259). In brief, a z-score of a candidate peptide is computed based on a numerical value and an associate range. If the z-score falls within the range of known CPP z-scores, the examined peptide is classified as a CPP.

Additional methods for the prediction of CPPs have been developed subsequently (see e.g. Sanders et al., Prediction of Cell Penetrating Peptides by Support Vector Machines, PLOS Computational Biology, 2011, 7(7), pp. 1-12, herein incorporated by reference) and a CPP database is available (Gautam et al., CPPSite: a curated database of cell penetrating peptides, Database, 2012, Article ID bas015 and http://crdd.osdd.net/raghava/cppsite/index.php, both herein incorporated by reference). Accordingly, any suitable CPP may find utility in the invention and, as discussed below, a variety of CPPs have already been identified and tested and could form the basis for determining and identifying new CPPs.

CPPs may be derived from naturally-occurring proteins which are able to translocate across cell membranes such as the Drosophila homeobox protein Antennapedia (a transcriptional factor), viral proteins such as the HIV-1 transcriptional factor TAT and the capsid protein VP22 from HSV-1, and/or they may be synthetically-derived, e.g. from chimeric proteins or synthetic polypeptides such as polyarginine. As noted above, there is not a single mechanism responsible for the transduction effect and hence the design of CPPs may be based on different structures and sequences. Cell penetrating peptides are also reviewed in Jarver et al. 2006 (Biochimica et Biophysica Acta 1758, pages 260-263) and Table 1 of U.S. Patent Application Publication No.: 2016/028972 lists various representative peptides. U.S. Pat. No. 6,645,501 (herein incorporated by reference) further describes various cell penetrating peptides which might be used.

In some cases, the polypeptide that enhances antimicrobial activity of an antibiotic is polymyxin antibiotic. In some cases, the polymyxin antibiotic is a polymyxin derivative. In some cases, a suitable polymyxin derivative is a polymyxin compound as described in U.S. Patent Application Publication No. 2016/0222061, which is hereby incorporated by reference in its entirety.

In some cases, suitable polymyxin derivative is a compound of Formula (VI):

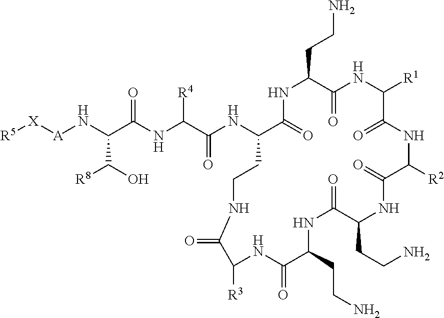

wherein:

—X— represents —C(O)—, —NHC(O)—, —OC(O)—, —CH₂— or —SO₂—; and

—R¹ together with the carbonyl group and nitrogen alpha to the carbon to which it is attached, is a phenylalanine, leucine or valine residue;

—R² together with the carbonyl group and nitrogen alpha to the carbon to which it is attached, is a leucine, iso-leucine, phenylalanine, threonine, valine or nor-valine residue;

—R³ together with the carbonyl group and nitrogen alpha to the carbon to which it is attached, is a threonine or leucine residue;

—R⁴ is C₁₆ alkyl substituted with one hydroxyl group or one amino group;

-A- is a covalent bond or an amino acid, such as an α-amino acid;

—R⁵ is G-L²-L¹-,

-G is selected from:

C₃₋₁₀ cycloalkyl,

C₂₋₁₂ alkyl,

C₅₋₁₂ aryl,

-L¹- is a covalent bond, C₁₋₁₂ alkylene or C₂₋₁₂ heteroalkylene,

-L²- is a covalent bond or C₄₋₁₀ heterocyclylene, with the proviso that -L¹- is not C₁₋₁₂ alkylene when -G is C₂₋₁₂ alkyl, and G-L²-L¹- is substituted with:

(i) one, two or three hydroxyl groups, or

(ii) one, two or three groups —NR⁶R⁷, or

(iii) one or two groups —NR⁶R⁷, and one, two or three hydroxyl groups,

with the proviso that (i), (ii) and (iii) are optional substituents when -L¹- is a nitrogen-containing C₂₋₁₂heteroalkylene and/or -L²- is a nitrogen-containing C₄₋₁₀ heterocyclylene, or —R⁵ is D-L¹-, where -D is C₄₋₁₀ heterocyclyl and -L¹- is as defined above, and D-L¹- is substituted with:

(i) one, two or three hydroxyl groups, or

(ii) one, two or three groups —NR⁶R⁷, or

(iii) one or two groups —NR⁶R⁷, and one, two or three hydroxyl groups,

with the proviso that (i), (ii) and (iii) are optional substituents when -L¹- is a nitrogen-containing C₂₋₁₂heteroalkylene and/or -D is a nitrogen-containing C₄₋₁₀ heterocyclyl,

each —R⁶ is independently hydrogen or C₁₋₄ alkyl;

each —R⁷ is independently hydrogen or C₁₋₄ alkyl;

or —NR⁶R⁷ is a guanidine group; or

when -G is C₃₋₁₀ cycloalkyl or C₅₋₁₂ aryl, —R⁶ and —R⁷ together with the nitrogen atom form a C₄₋₁₀ heterocycle; and

where an aryl group is present in —R⁵ it is independently optionally substituted one or more substituents selected from —C₁₋₁₀ alkyl, such as —C₁₋₄ alkyl, halo, —CN, —NO₂, —CF₃, optionally —C(O)R¹⁰, —NR¹⁰C(O)R¹⁰, —OCF₃, —CON(R¹⁰)₂, —COOR⁹, —OCOR¹⁰, —NR¹⁰COOR¹⁰, —OCON(R¹⁰)₂, —NR¹⁰CON(R¹⁰)₂, —OR⁹, —SR⁹, —NR¹⁰SO₂R¹⁰, —SO₂N(R¹⁰)₂ and —SO₂R¹⁰ here each —R⁹ is independently —C₁₋₁₀ alkyl, such as —C₁₋₄ alkyl and each —R¹⁰ is independently —H or —C₁₋₁₀ alkyl, such as —C₁₋₄ alkyl;

and optionally where an alkyl, cycloalkyl, or heterocyclyl group is present in —R⁵ it is independently optionally substituted with one or more substituents selected from —C₁₋₁₀ alkyl, such as —C₁₋₄alkyl, halo, —CN, —NO₂, —CF₃, —C(O)R¹⁰, —NR¹⁰C(O)R¹⁰, —OCF₃, CON(R¹⁰)₂, —COOR⁹, —OCOR¹, —NR¹⁰COOR¹⁰, —OCON(R¹⁰)₂, —NR¹⁰CON(R¹⁰)₂, —OR⁹, —SR⁹, —NR¹SO₂R¹⁰, —SO₂N(R¹⁰)₂ and —SO₂R¹⁰ where each —R⁹ is independently —C₁₋₁₀ alkyl, such as —C₁₋₄ alkyl and each —R¹⁰ is independently —H or —C₁₋₁₀ alkyl, such as —C₁₋₄ alkyl, except that alkyl is not substituted with alkyl;

—R⁸ is hydrogen or methyl.

In some cases, the compound of Formula (VI) does not encompass deacylated polymyxin compounds. In some cases, the compound of Formula (VI) does not encompass the polymyxin derivatives described by Katsuma et al. (Chem. Pharm. Bull. 2009, 57, 332).

In some cases, suitable polymyxin derivative is a compound of Formula (VII). In some cases, derivatives of the compound of Formula (VII) are compounds of formula (LXXVI), (LXXVII), (LXXVIII), (LXXIX). In some cases, the derivatives of compound of Formula (VII) are compounds with Formulas (LXXVI), (LXXVII), (LXXVIII), (LXXIX) in combination with (LXXX), (LXXXI), and (LXXXII).

In some cases, the derivative of the compound of Formula (VII) is a compound of formula (LXXVI). The compound of formula (LXXVI) are compounds where:

—R⁵ is G-L²-L¹-, and

-G is C₅₋₁₂ aryl,

-L¹- is a covalent bond, C₁₋₁₂ alkylene or C₂₋₁₂ heteroalkylene,

-L²- is a covalent bond or C₄₋₁₀ heterocyclylene,

—R⁵ is substituted with:

(i) one, two or three hydroxyl groups, or

(ii) one, two or three groups —NR⁶R⁷, or

(iii) one or two groups —NR⁶R⁷, and one, two or three hydroxyl groups,

with the proviso that (i), (ii) and (iii) are optional substituents when -L¹- is a nitrogen-containing C₂₋₁₂heteroalkylene and/or -L²- is a nitrogen-containing C₄₋₁₀ heterocyclylene, and the aryl group is independently optionally substituted with one or more substituents selected from —C₁₋₄ alkyl, halo, —CN, —NO₂, —CF₃, —NR¹⁰C(O)R¹⁰, —OCF₃, —CON(R¹⁰)₂, COOR⁹, —OCOR¹, —NR¹⁰COOR¹⁰, —OCON(R¹⁰)₂, —NR¹⁰CON(R¹⁰)₂, —OR⁹, —SR⁹, NR¹⁰SO₂R¹⁰, —SO₂N(R¹⁰)₂ and —SO₂R¹⁰ where each —R⁹ is independently —C₁₋₁₀ alkyl, such as —C₁₋₄ alkyl and each —R¹⁰ is independently —H or —C₁₋₁₀ alkyl, such as —C₁₋₄ alkyl;

and R¹, R², R³, R⁴, R⁶, R⁷, R⁸ have the same meanings as the compounds of formula (I) above. Additionally -A- and —X— have the same meanings as the compounds of formula (I) above. Optionally, —R⁵—X— together are not Phe, His, Trp or Tyr, such as L-Phe, L-His, L-Trp and L-Tyr, for example when -A- is a covalent bond. Optionally, —R⁵—X— together are not Phe, and Trp, such as L-Phe and L-Trp, for example when -A- is a covalent bond.

In some cases, the derivative of compound of Formula (VII) is a compound of formula (LXXVII). The compound of formula (LXXVII) are compounds where:

—R⁵ is G-L²-L¹-, and -G is C₃₋₁₀ cycloalkyl,

-L¹- is a covalent bond, C₁₋₁₂ alkylene or C₂₋₁₀ heteroalkylene,

-L²- is a covalent bond or C₄₁₂ heterocyclylene,

with the proviso that -L²- is a covalent bond only when -L¹- is C₂₋₁₀ heteroalkylene,

—R⁵ is substituted with:

(i) one, two or three hydroxyl groups, or

(ii) one, two or three groups —NR⁶R⁷, or

(iii) one or two groups —NR⁶R⁷, and one, two or three hydroxyl groups,

with the proviso that (i), (ii) and (iii) are optional substituents when -L¹- is a nitrogen-containing C₂₋₁₂heteroalkylene and/or -L²- is a nitrogen-containing C₄₋₁₀ heterocyclylene,

and optionally the cycloalkyl group is independently optionally substituted with one or more substituents selected from —C₁₋₁₀ alkyl, such as —C₁₋₄ alkyl, halo, —CN, —NO₂, —CF₃, —C(O)R¹⁰, —NR¹⁰C(O)R¹⁰, —OCF₃, —CON(R¹⁰)₂, —COOR⁹, —OCOR¹, —NR¹⁰COOR¹⁰, —OCON(R¹⁰)₂, —NR¹⁰CON(R¹⁰)₂, —OR⁹, —SR⁹, —NR¹⁰SO₂R¹⁰, —SO₂N(R¹⁰)₂ and —SO₂R¹⁰where each —R⁹ is independently —C₁₋₁₀ alkyl, such as —C₁₋₄ alkyl and each —R¹⁰ is independently —H or —C₁₋₁₀ alkyl, such as —C₁₋₄ alkyl, except that alkyl is not substituted with alkyl, and R¹, R², R³, R⁴, R⁶, R⁷, R⁸ have the same meanings as the compounds of formula (I) above. Additionally -A- and —X— have the same meanings as the compounds of formula (I) above.

In some cases, the derivative of compound of Formula (VII) is a compound of formula (LXXVIII). The compound of formula (LXXVIII) are compounds where:

—R⁵ is G-L²-L¹-, where -G is C₃₋₁₀ cycloalkyl or C₂₋₁₂alkyl,

-L¹- is a covalent bond or C₁₋₁₂ alkylene,

-L²- is a covalent bond, with the proviso that -L¹- is not C₁₋₁₂ alkylene when -G is C₂₋₁₂ alkyl, —R⁵ is substituted with:

(i) two or three groups —NR⁶R⁷, or

(ii) two groups —NR⁶R⁷, and one, two or three hydroxyl groups;

and optionally the alkyl or cycloalkyl group is independently optionally substituted with one or more substituents selected from —C₁₋₁₀ alkyl, such as —C₁₋₄ alkyl, halo, —CN, —NO₂, —CF₃, —C(O)R¹⁰, —NR¹⁰C(O)R¹⁰, —OCF₃, —CON(R¹⁰)₂, —COOR⁹, —OCOR¹, —NR¹⁰COOR¹⁰, —OCON(R¹⁰)₂, —NR¹⁰CON(R¹⁰)₂, —OR⁹, —SR⁹, —NR¹⁰SO₂R¹⁰, —SO₂N(R¹⁰)₂ and —SO₂R¹⁰where each —R⁹ is independently —C₁₋₁₀ alkyl, such as —C₁₋₄ alkyl and each —R¹⁰ is independently —H or —C₁₋₁₀ alkyl, such as —C₁₋₄ alkyl, except that alkyl is not substituted with alkyl,

and R¹, R², R³, R⁴, R⁶, R⁷, R⁸ have the same meanings as the compounds of formula (I) above. Additionally -A- and —X— have the same meanings as the compounds of formula (I) above. Optionally, —R⁵—X— together are not Lys, Dap, Arg, Dab, and Drg, such as L-Lys, L-Dap, L-Arg, L-Dab, and L-Drg, for example where -A- is a covalent bond.

In some cases, the derivative of compound of Formula (VII) is a compound of formula (LXXIX). The compound of formula (LXXIX) are compounds where:

—R⁵ is D-L¹-, where D-L¹- is substituted with:

(i) one, two or three hydroxyl groups, or

(ii) one, two or three groups —NR⁶R⁷, or

(iii) one or two groups —NR⁶R⁷, and one, two or three hydroxyl groups; -L¹- is a covalent bond, C₁₋₁₂ alkylene or C₂₋₁₂ heteroalkylene,

with the proviso that (i), (ii) and (iii) are optional substituents when -L¹- is a nitrogen-containing C₂₋₁₂heteroalkylene,

and optionally the heterocyclyl group is independently optionally substituted with one or more substituents selected from —C₁₋₁₀ alkyl, such as —C₁₋₄ alkyl, halo, —CN, —NO₂, —CF₃, —C(O)R¹⁰, —NR¹⁰C(O)R¹⁰, —OCF₃, —CON(R¹⁰)₂, —COOR⁹, —OCOR¹, —NR¹⁰COOR¹⁰, —OCON(R¹⁰)₂, —NR¹⁰CON(R¹⁰)₂, —OR⁹, —SR⁹, —NR¹⁰SO₂R¹⁰, —SO₂N(R¹⁰)₂ and —SO₂R¹⁰where each —R⁹ is independently —C₁₋₁₀ alkyl, such as —C₁₋₄ alkyl and each —R¹⁰ is independently —H or —C₁₋₁₀ alkyl, such as —C₁₋₄ alkyl, except that alkyl is not substituted with alkyl,

and R¹, R², R³, R⁴, R⁶, R⁷, R⁸ have the same meanings as the compounds of formula (I) above. Additionally -A-, -D, and —X— have the same meanings as the compounds of formula (I) above.

In some cases, the derivative of compound of Formula (VII) is a compound of formula (LXXX). The compound of formula (LXXX) are compounds where:

-A- is an amino acid, such as an α-amino acid, and R¹, R², R³, R⁴, R⁵, R⁶, R⁷, R⁸, and —X— have the same meanings as the compounds of formula (I) above. It is noted that the compounds described by Katsuma et al. (Chem. Pharm. Bull. 2009, 57, 332) are Polymyxin B decapeptides. However, these compounds do not have the N terminal modifications that are present in the compounds of formula (LXXX).

In some cases, the derivative of compound of Formula (VII) is a compound of formula (LXXXI). The compound of formula (LXXXI) are compounds where:

-A- is a covalent bond, and R¹, R², R³, R⁴, R⁵, R⁶, R⁷, R⁸, and —X— have the same meanings as the compounds of formula (I) above, with the proviso that —X— and —R⁵ together are not an L-α-amino acid residue. In one embodiment, —X— and —R⁵ together are not L-Lys, L-Arg, L-Dap (L-α,β-diaminopropionic acid), L-Ser, L-Dab (L-α,γ-diaminobutyric acid), L-Dgp (L-α,β-diguanidinopropanoyl) or L-Abu.

In some cases, where —X— and —R⁵ together are an α-amino acid, that α-amino acid is a D-α-amino acid residue.

It is noted that the compounds described by Katsuma et al. (Chem. Pharm. Bull. 2009, 57, 332) are des-fatty Polymyxin B decapeptides. The amino acid at the 1-position in the decapeptide is a L-α-amino acid, for example L-Lys, L-Arg, L-Dap (L-α,β-diaminopropionic acid), or L-Ser. The compounds of formula (LXXXI) do not encompass the compounds of Katsuma et al., as such amino acids are excluded from the definition of X— and —R⁵ (when -A- is a covalent bond).

The compounds described by Sato et al. (Peptide Science 2007, 307) are des-fatty Polymyxin B decapeptides. The amino acid at the 1-position in the decapeptide is a L-α-amino acid, for example L-Dab, L-Dap, L-Dgp and L-Ser. The compounds of formula (LXXXI) do not encompass the compounds of Sato et al., as such amino acids are excluded from the definition of X— and —R⁵ (when -A- is a covalent bond).

WO 2009/098357 describes a control compound NAB 705, which is a decapeptide comprising a Polymyxin B nonapeptide having an L-Abu residue at the N terminal. The compounds of formula (LXXXI) do not encompass the compound of WO 2009/098357, as the amino acid is excluded from the definition of —X— and —R⁵ (when A- is a covalent bond). NAB 705 is also described in WO 2008/017734. The compounds of Katsuma et al. and Sato et al. are not described for use in combination with an active agent.

In some cases, the derivative of compound of Formula (VII) is a compound of formula (LXXXII). The compound of formula (LXXXII) are compounds where:

—R⁴, together with the carbonyl group and nitrogen alpha to the carbon to which it is attached, is not Dab, for example is not (S)-Dab. Thus, —R⁴ is not —CH₂CH₂NH₂ in an (S)-configuration about the carbon to which is attached. In this embodiment, -A-, R¹, R², R³, R⁵, R⁶, R⁷, R⁸, and —X— have the same meanings as the compounds of formula (I) above.

In some cases, —R⁴ is C₁ alkyl or C₃₆ alkyl substituted with one hydroxyl group or one amino group.

In some cases, —R⁴ is C₁ alkyl substituted with one hydroxyl group or one amino group.

In some cases, —R⁴, together with the carbonyl group and nitrogen alpha to the carbon to which it is attached, is Dap (α,β-diaminopropionic acid), such as (S)-Dap.

The compounds of formula (LXXXII) are compounds that do not share with Polymyxin B the amino acid residue at position 3. The work by Sato et al. and Katsuma et al., for example, is limited to the description of Polymyxin B and Colistin compounds, which possess a (S)-Dab residue at position 3.

WO 2012/168820 describes polymyxin compounds where the amino acid at position 3 has an altered side chain in comparison to Polymyxin B. WO 2012/168820 does not describe compounds having the N terminal groups (i.e. the group —X—R⁵) that are described in the present case.

Where A is a covalent bond, R¹ (together with associated groups) is D-phenylalanine, R² (together with associated groups) is L-leucine, R³ (together with associated groups) is L-threonine, R⁴ (together with associated groups) is L-α,γ-diaminobutyric acid; and R⁸ is methyl (and together with the associated groups is L-threonine), the compound is a polymyxin nonapeptide derivative having amino acids 2-10 of polymyxin B (polymyxin B nonapeptide). Further, where A is L-α,γ-diaminobutyric acid, the compound is a polymyxin derivative having amino acids 1-10 of polymyxin B.

Similarly, where A is a covalent bond, R¹ (together with associated groups) is D-leucine, R² (together with associated groups) is L-leucine, R³ (together with associated groups) is L-threonine, R⁴ (together with associated groups) is L-α,γ-diaminobutyric acid; and R⁸ is methyl (and together with the associated groups is L-threonine), the compound is a polymyxin nonapeptide having amino acids 2-10 of polymyxin E (colistin nonapeptide). Further, where A is L-α,γ-diaminobutyric acid, the compound is a polymyxin derivative having amino acids 1-10 of polymyxin E (colistin).

In some cases, the suitable polymyxin derivative is a polymyxin compound of Formula (vi). In some cases, the suitable polymyxin derivative is a polymyxin compound of Formula (vii). In some cases, the polymyxin derivatives with Formula (vi) or Formula (vii) are N terminal derivatives of the polymyxin series of compounds. In some cases, the core of a suitable polymyxin derivative is a deacylated version of a polymyxin compound or a nonapeptide version of a polymyxin compound, such as a deacylated polymyxin B nonapeptide (PMBN) or a deaclyated Colisin.

In some cases, the polymyxin compounds of Formula (vi) and Formula (vii) may be used together with certain compounds in the rifamycin family. The rifamycin family includes isolates rifamycin A, B, C, D, E, S and SV, and synthetically derivatised versions of these compounds, such as rifampicin (rifampin), rifabutin, rifalazil, rifapentine, and rifaximin, and pharmaceutically acceptable salts and solvates thereof. In some cases, the polymyxin derivatives of Formula (vi) and Formula (vii) may be used together with certain compounds in the rifamycin family to treat microbial infections.

In some cases, the suitable polymyxin derivatives of formula (vi) and (vii) may be used together with certain compounds in the meropenem family to treat microbial infections. In one embodiment, the meropenem family includes meropenem, doripenem, imipenem, ertapenem, biapenem, tomopenem, and panipenem, and pharmaceutically acceptable salts and solvates thereof.

In some cases, the suitable polymyxin derivative of Formula (vii) may also be used together with the second agents above. The polymyxin derivative of Formula (vii) may additionally be used together with other second agents such as vancomycin, fosfomycin, rifamycin, a beta-lactam (such as a cephalosporin or carbapenem), an aminoglycoside, a macrolide, a tetracyline, a lipopeptide, and/or an oxazolidinone.

In some cases, the polymyxin derivative of Formula (vii) may additionally be used together with vancomycin or fosfomycin. In other cases, the second agent is not vancomycin, fosfomycin, rifamycin, a beta-lactam (such as a cephalosporin or carbapenem), an aminoglycoside, a macrolide, a tetracyline, a lipopeptide, an oxazolidinone and/or an anti-inflammatory such as a steroid.

### Polymyxin B Nonapeptide

In some cases, the antimicbrial agent is a polypeptide that enhances antimicrobial activity of an antibiotic. In some cases, the polypeptide that enhances antimicrobial activity of an antibiotic is polymyxin B nonapeptide, NAB7061, or NAB741. In some cases, the suitable polymyxin derivative is a Polymyxin B nonapeptide.

Peptides such as polymyxin B and the related colistin (polymyxin E) have been administered to humans as antibacterial agents. However, their use has been previously limited because of their toxicity. These peptides comprise a seven amino acid cyclic peptide attached to an exocyclic three amino acid chain, wherein the N-terminal amine of the exocyclic chain is linked to a “side chain” or “tail”. The tail is an acyl group.

Renal toxicity has been observed with the recommended dosing of polymyxin B in some patients. Neurotoxicity or neuropathy has also been observed in patients with compromised renal functions, with an overall incidence of 7.3% reported in one large study with colistin (see, e.g., Evans, et al. (1999) Ann. Pharmacother. 33:960-967). When the acyl exocyclic chain and the adjacent N-terminal 2,4-diaminobutanoic acid (Dab) residue are enzymatically removed from polymyxin, this yields the corresponding polymyxin nonapeptide. The in vivo toxicity of the nonapeptide of polymyxin B is significantly less than that of polymyxin B itself (see, e.g., Kimura, et al. (1992) J. Antibiot., 45, 742-749). The toxicity of the nonapeptide in cell culture is reduced by about 100-fold relative to polymyxin B. However, the antibacterial activity of the nonapeptide is also reduced by about 2-64 fold relative to polymyxin B (see, e.g., Duwe, et al. (1986) Antimnicrob. Agents Chemother, 30:340-341).

Polymyxin B nonapeptides (PMBN) lack the fatty acyl tail and the N-terminal amino acyl residue but retains the 5 total positive charges. PMBN retains the ability to permeabilize the outer membrane (OM) of Gram-negative bacteria (U.S. Pat. No. 4,510,132). Accordingly, even though it lacks the direct antibacterial activity (i.e. the ability to inhibit bacterial growth), it is able to sensitize (i.e. make sensitive or, as also termed, make susceptible) the bacteria to many antibacterial agents such as hydrophobic antibiotics as well as large antibiotics and some other noxious agents. PMBN also sensitizes bacteria to the bactericidal activity of the human complement system, present in fresh human serum as a first-line defence system against invaders. Furthermore, it sensitizes the bacteria to the joint bactericidal activity of serum complement and human polymorphonuclear white cells, PMBN resembles PMEN in being less toxic in the acute toxicity assay in mice than unmodified polymyxins. In further toxicological assays, several criteria proved PBMN to be less toxic than its parent compound, but this polymyxin derivative alone was still judged to be too nephrotoxic for clinical use.

PMBN carries five (5) positive charges. Subsequent studies revealed, quite expectedly, that PMEN, also carrying five (5) positive charges as well as deacylpolymyxin B and deacylpolymyxin E, both carrying six (6) positive charges are potent agents to sensitize bacteria to other antibiotics. In addition, it has been shown that a structurally further reduced derivative polymyxin B octapeptide (PMBO) retains a very effective permeabilizing activity while polymyxin B heptapeptide (PMBH) is less active. PMBN, PMEN and PMBO have five (5) positive charges while PMBH has only four (4) positive charges.

A shortened polymyxin B derivative octanoyl polymyxin B heptapeptide can be used. The attachment of the octanoyl residue to the N-terminus of the residue R⁴ of the polymyxin B heptapeptide results in a compound having only three (3) positive charges. Octanoyl polymyxin B heptapeptide inhibits the growth of bacteria only at a very high concentration (128 μg/ml), whereas the other derivatives such as octanoyl polymyxin B octapeptide and octanoyl polymyxin B nonapeptide, both having four charges (4) were very potent agents to inhibit bacterial growth.

U.S. Patent Publication No.: 2006004185 disclosed certain polymyxin derivatives and intermediates that can be used. The antibacterial compounds described possessed four (4) or five (5) positive charges.

In some cases, the suitable Polymyxin B nonapeptide has the structure shown below:

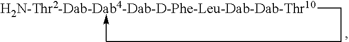

wherein:

positions 2, 4 and 10 are indicated (with reference to the numbering system used for the Polymyxin B decapeptide), and the amino acid residues are in the L-configuration, unless indicated.

In some cases, the suitable polymyxin derivatives are derivatives of polymyxin B nonapeptide, where (i) the N terminal amino group, —NH₂, is replaced with the group —NH-A-X—R⁵ or —NH—X—R¹⁵ as described herein and optionally (ii) the amino acid residues at 2, 3, 6, 7 and 10 positions are substituted with another amino acid residue.

In some cases, the polymyxin derivatives are represented by the formula (vi) or (vii) where the amino acids at positions 2, 3, 6, 7 or 10 are determined by the nature of the groups R⁸, R⁴, R¹, R² and R³ respectively.

In some cases, the polymyxin derivatives are are biologically active.

In some cases, the polymyxin derivative of Formula (vi) or Formula (vii) is a compound in which one or more, for example, from 1 to 5, such as 1, 2, 3 or 4 amino acids are substituted by another amino acid. The amino acid may be at a position selected from positions 2, 3, 6, 7 or 10 (referring to the numbering of residues used in polymyxin B). In some cases, the substitution may be for another amino acid or for a stereoisomer.

Examples of polymyxin derivatives include, but are not limited to Polymyxin B Nonapeptide, Tetra-(Boc) Polymyxin B Nonapeptide, Colistin (Polymyxin E) Nonapeptide, Tetra-(Boc) Colistin (Polymyxin E) Nonapeptide, Tri-(Boc) Polymyxin B Heptapeptide, Penta-(Boc) Polymyxin B Decapeptide, Thr(O-′Bu) Tetra-(N-Boc) Polymyxin B Nonapeptide, Thr(O-′Bu) Penta-(N-Boc) Polymyxin B decapeptide. Examples of polymyxin B nonapeptides derivatives and methods of preparation of polymyxin B nonapeptide derivatives can be found in U.S. Patent Publication No. 2016/0222061, which is hereby incorporated by reference in its entirety. The structures of such polymyxin derivatives are shown below.

In some cases, a suitable polymyxin derivative is a compound of Formula (VIII):

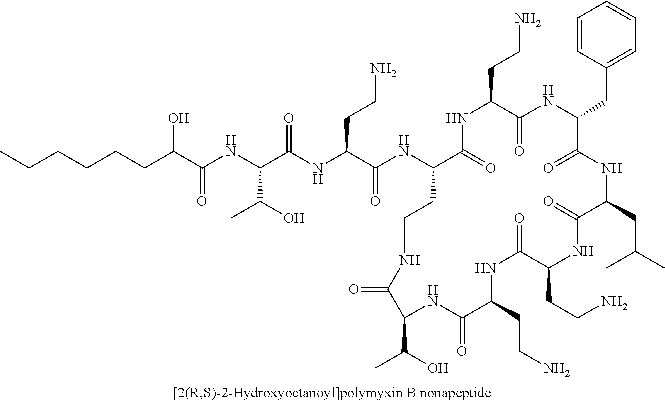 [SMILES: CCCCCCC(O)C(=O)N[C@H](C(=O)N[C@@H](CCN)C(=O)N[C@H]1CCNC(=O)[C@H](C(C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)C(C)O]

In some cases, a suitable polymyxin derivative is a compound of Formula (IX):

| Formula | Mass |
|---|---|
| C49H85N15O12 | 1075.65 |

6-Aminohexanoyl polymyxin B nonapeptide

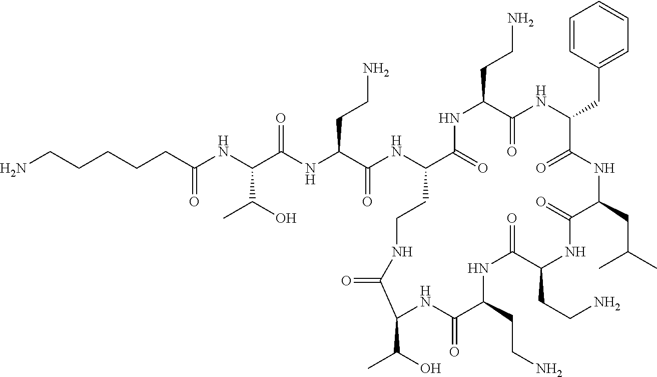 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CCCCCN)C(C)O)CCNC(=O)[C@H](C(C)O)NC(=O)[C@H](CCN)NC(=O)[C@H](CCN)NC1=O]

In some cases, a suitable polymyxin derivative is a compound of Formula (X):

| Formula | Mass |
|---|---|
| C46H79N15O12 | 1033.60 |

3-Aminopropanoyl polymyxin B nonapeptide

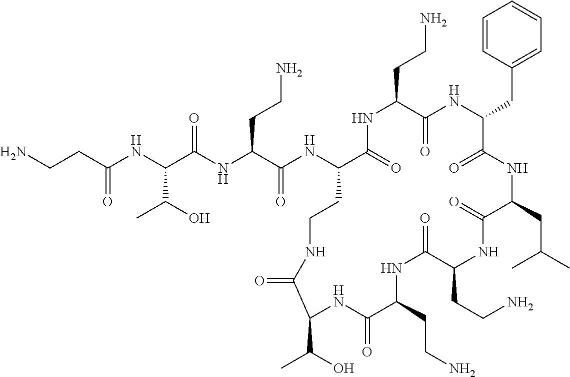 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CCN)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XI):

| Formula | Mass |
|---|---|
| C47H81N15O12 | 1047.62 |

4-Aminobutanoyl polymyxin B nonapeptide

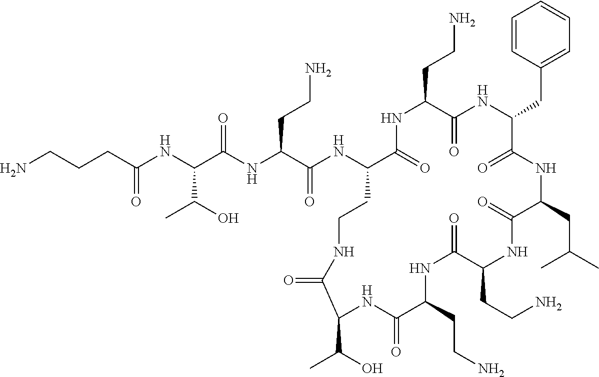 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CCCN)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XII):

| Formula | Mass |
|---|---|
| C50H79N15O12 | 1081.60 |

4-Aminobenzoyl polymyxin B nonapeptide

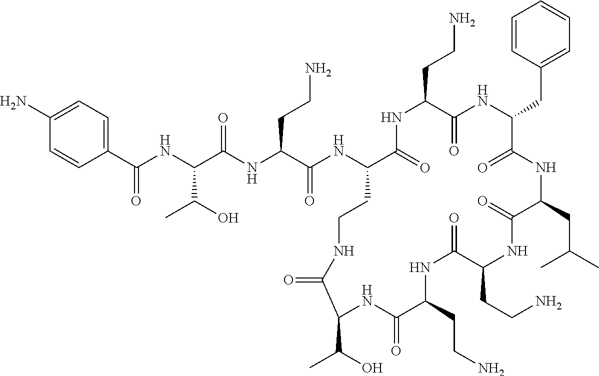 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)c2ccc(N)cc2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XIII):

| Formula | Mass |
|---|---|
| C48H83N15O12 | 1061.63 |

5-Aminopentanoyl polymyxin B nonapeptide

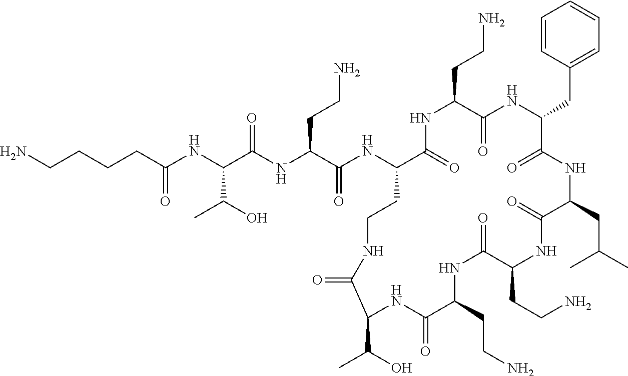 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CCCCN)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XIV):

| Formula | Mass |
|---|---|
| C49H83N15O12 | 1073.63 |

(1R,S/2R,S)-2-Aminocyclopentane-carbonyl polymyxin B nonapeptide

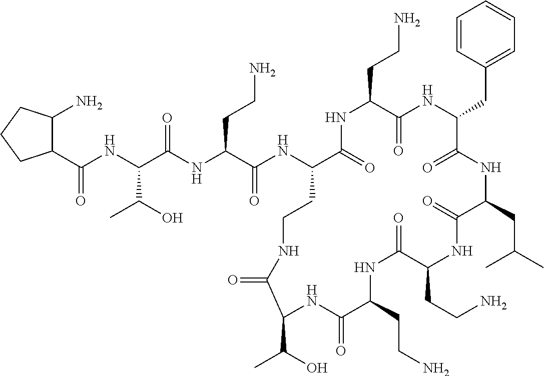 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)C2CCCC2N)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XV):

| Formula | Mass |
|---|---|
| C45H76N14O13 | 1020.57 |

Hydroxyacetyl polymyxin B nonapeptide

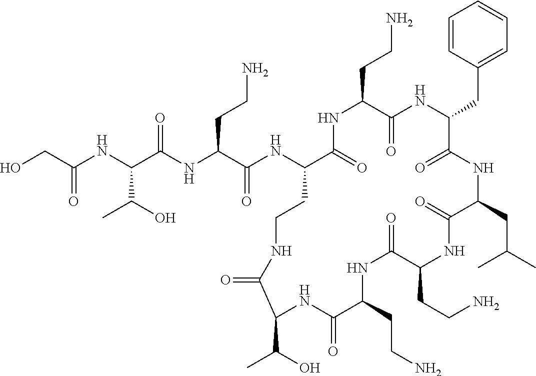 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CO)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XVI):

| Formula | Mass |
|---|---|
| C48H81N15O12 | 1059.62 |

[3(R,S)-Pyrrolidine-3-carbonyl]polymyxin B nonapeptide

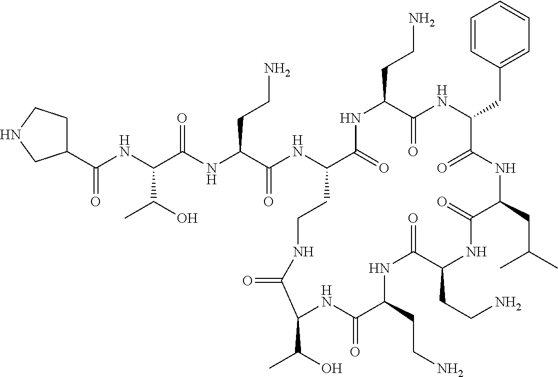 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)C2CCNC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XVIII):

| Formula | Mass |
|---|---|
| C52H89N15O12 | 1115.68 |

[3(R,S)-3-Amino-3-cyclohexane-propanoyl]Polymyxin B nonapeptide

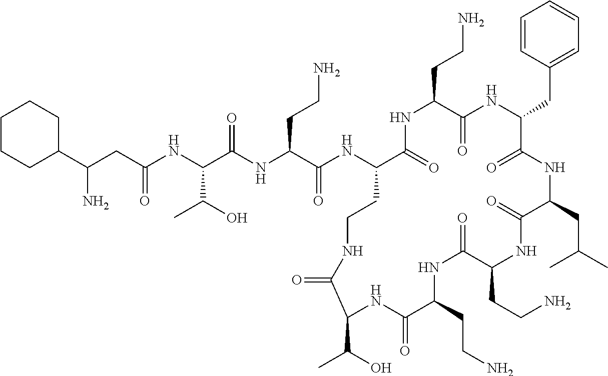 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CC(N)C2CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XVIII):

| Formula | Mass |
|---|---|
| C49H85N15O12 | 1075.65 |

4-(N,N-dimethylamino)-butanoyl polymyxin B nonapeptide

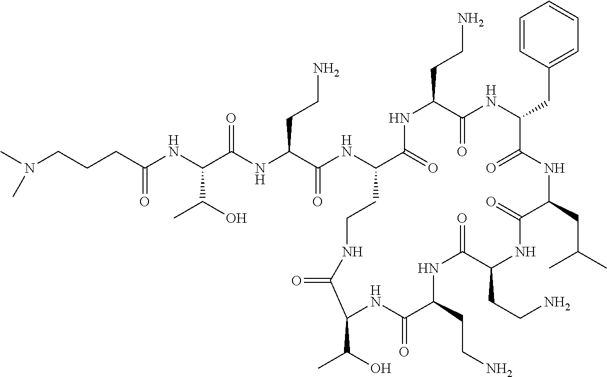 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CCCN(C)C)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XIX):

| Formula | Mass |
|---|---|
| C50H85N15O14S | 1151.61 |

3-(1,1-dioxo-thiomorpholine-4-yl)propanoyl Polymyxin B nonapeptide

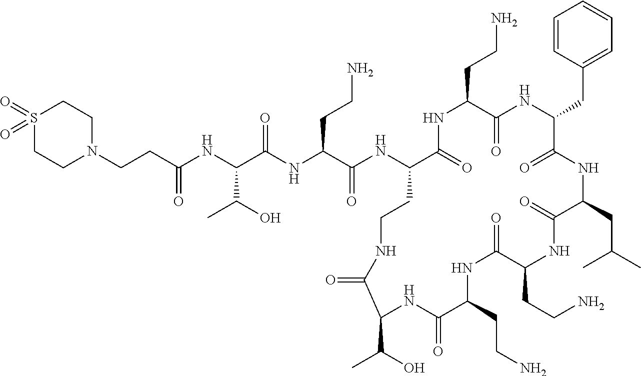 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CCN2CCS(=O)(=O)CC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XX):

| Formula | Mass |
|---|---|
| C50H87N15O12 | 1089.67 |

7-Aminoheptanoyl polymyxin B nonapeptide

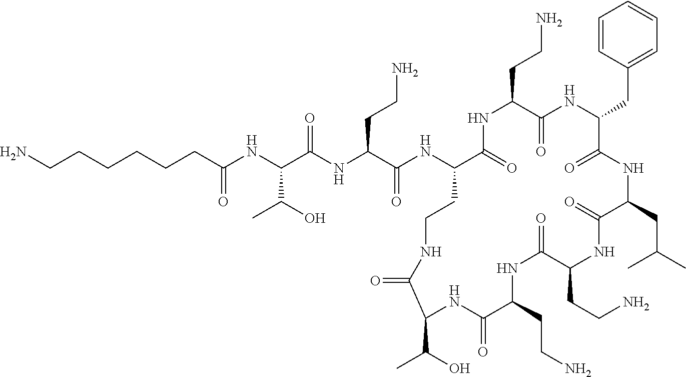 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CCCCCCN)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XXI):

| Formula | Mass |
|---|---|
| C51H87N15O13 | 1117.66 |

4-Morpholinylbutanoyl polymyxin B nonapeptide, trifluoroacetate salt

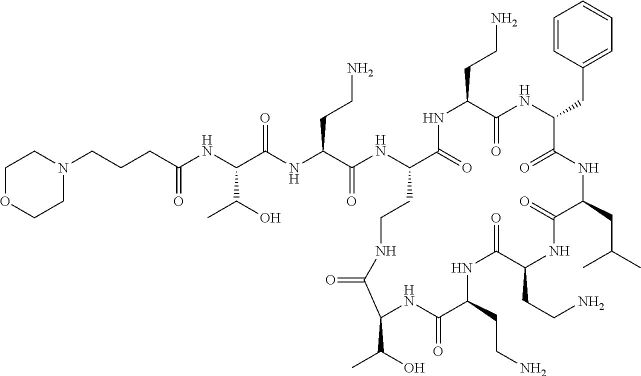 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CCCN2CCOCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XXII):

| Formula | Mass |
|---|---|
| C47H80N14O13 | 1048.60 |

3(RS)-3-Hydroxybutanoyl polymyxin B nonapeptide

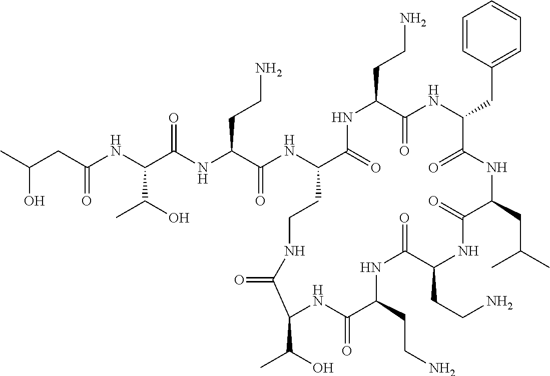 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CC(C)O)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XXIII):

| Formula | Mass |
|---|---|
| C48H83N15O12 | 1061.63 |

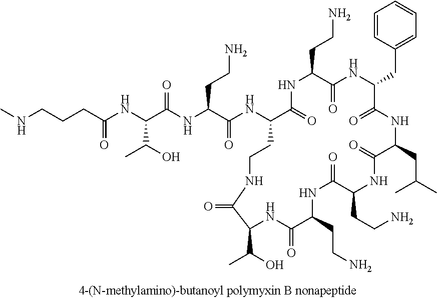 [SMILES: CNCCCC(=O)N[C@H](C(=O)N[C@@H](CCN)C(=O)N[C@H]1CCNC(=O)[C@H](C(C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)C(C)O]

In some cases, a suitable polymyxin derivative is a compound of Formula (XXIV):

| Formula | Mass |
|---|---|
| C50H85N15O12 | 1087.65 |

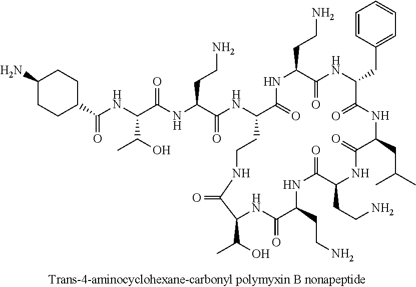

In some cases, a suitable polymyxin derivative is a compound of Formula (XXV):

| Formula | Mass |
|---|---|
| C51H79BrN14O13 | 1176.5, |
| | 1174.5 |

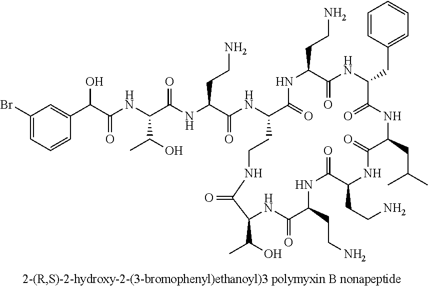 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)C(O)c2cccc(Br)c2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XXVI):

| Formula | Mass |
|---|---|
| C48H90N14O13 | 1070.68 |

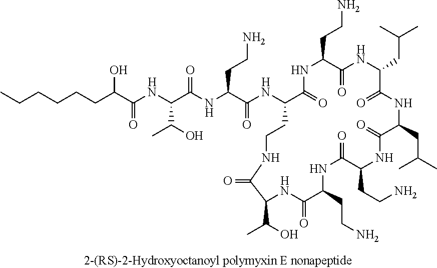 [SMILES: CCCCCCC(O)C(=O)N[C@H](C(=O)N[C@@H](CCN)C(=O)N[C@H]1CCNC(=O)[C@H](C(C)O)NC(=O)[C@H](CCN)NC(=O)[C@H](CCN)NC(=O)[C@H](CC(C)C)NC(=O)[C@@H](CC(C)C)NC(=O)[C@H](CCN)NC1=O)C(C)O]

In some cases, a suitable polymyxin derivative is a compound of Formula (XXVII):

| Formula | Mass |
|---|---|
| C50H85N15O12 | 1087.65 |

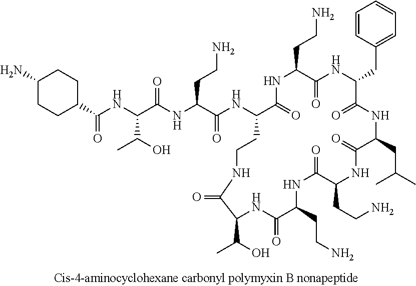

In some cases, a suitable polymyxin derivative is a compound of Formula (XXVIIII):

| Formula | Mass |
|---|---|
| C49H85N15O12 | 1075.65 |

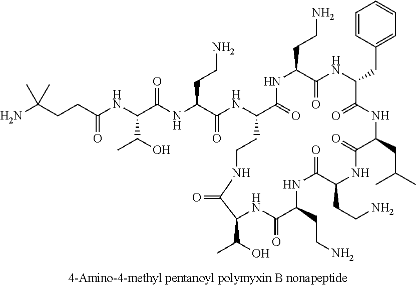 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CCC(C)(C)N)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XXIX):

| Formula | Mass |
|---|---|
| C50H87N15O12 | 1089.67 |

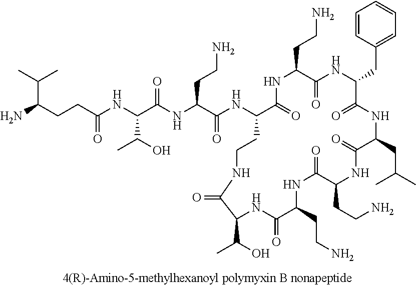 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CC[C@@H](N)C(C)C)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XXX):

| Formula | Mass |
|---|---|
| C50H85N15O12 | 1087.65 |

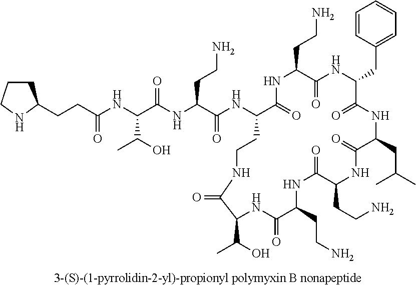 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CC[C@@H]2CCCN2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxxi):

| Formula | Mass |
|---|---|
| C48H83N15O12 | 1061.63 |

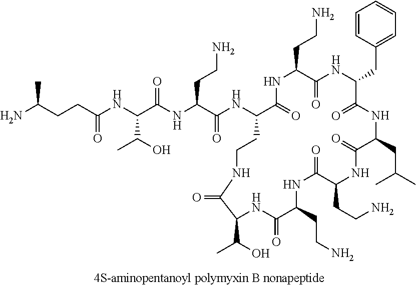 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CC[C@H](C)N)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxxii):

| Formula | Mass |
|---|---|
| C50H84N14O13 | 1088.63 |

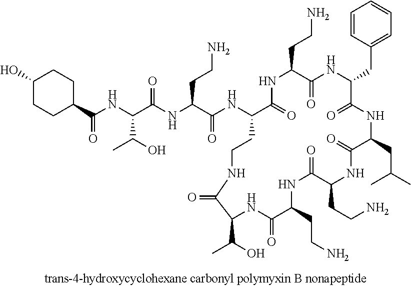 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@H]2CC[C@H](O)CC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxxiii):

| Formula | Mass |
|---|---|
| C46H78N14O13 | 1034.59 |

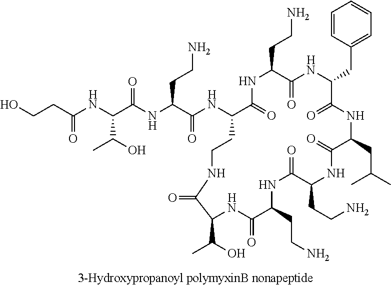 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CCO)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxxiv):

| Formula | Mass |
|---|---|
| C51H86N14O13 | 1102.65 |

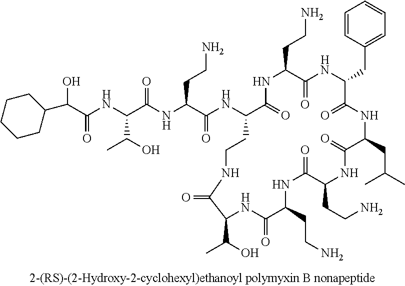 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)C(O)C2CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxxv):

| Formula | Mass |
|---|---|
| C52H83N15O12 | 1109.63 |

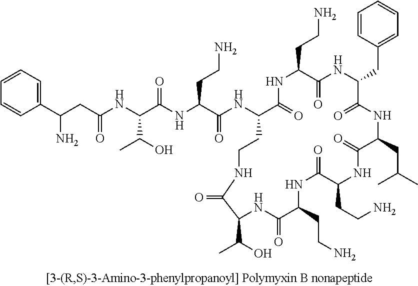 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CC(N)c2ccccc2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxxvi):

| Formula | Mass |
|---|---|
| C49H83N15O12 | 1073.63 |

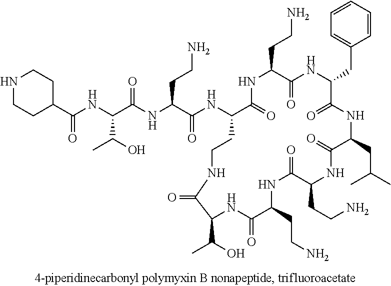 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)C2CCNCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxxvii):

| Formula | Mass |
|---|---|
| C49H91N15O12 | 1081.70 |

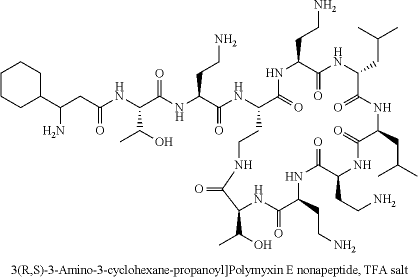 [SMILES: CC(C)C[C@@H]1NC(=O)[C@@H](CC(C)C)NC(=O)[C@H](CCN)NC(=O)[C@@H](NC(=O)[C@H](CCN)NC(=O)[C@@H](NC(=O)CC(N)C2CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxxviii):

| Formula | Mass |
|---|---|
| C48H88N14O13 | 1068.67 |

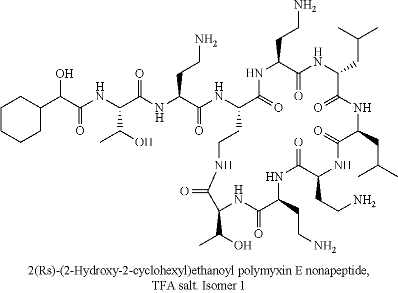 [SMILES: CC(C)C[C@@H]1NC(=O)[C@@H](CC(C)C)NC(=O)[C@H](CCN)NC(=O)[C@@H](NC(=O)[C@H](CCN)NC(=O)[C@@H](NC(=O)C(O)C2CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxxix):

| Formula | Mass |
|---|---|
| C48H88N14O13 | 1068.67 |

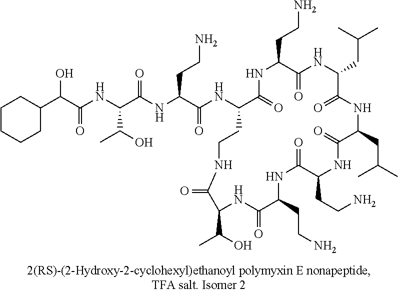 [SMILES: CC(C)C[C@@H]1NC(=O)[C@@H](CC(C)C)NC(=O)[C@H](CCN)NC(=O)[C@@H](NC(=O)[C@H](CCN)NC(=O)[C@@H](NC(=O)C(O)C2CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (XXL):

| Formula | Mass |
|---|---|
| C50H85N15O12 | 1087.65 |

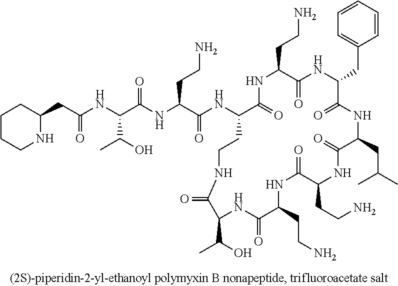 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)C[C@@H]2CCCCN2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxli):

| Formula | Mass |
|---|---|
| C49H83N15O12 | 1073.63 |

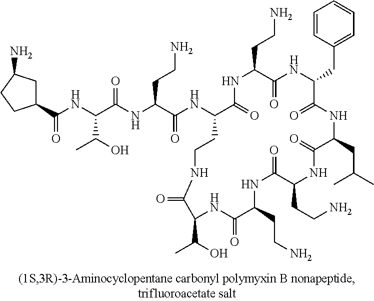 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@H]2CC[C@@H](N)C2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxlii):

| Formula | Mass |
|---|---|
| C49H83N15O12 | 1073.63 |

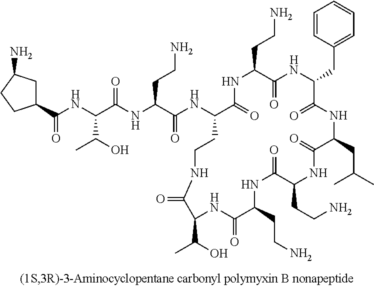 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@H]2CC[C@@H](N)C2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxliii):

| Formula | Mass |
|---|---|
| C48H81N15O13 | 1075.61 |

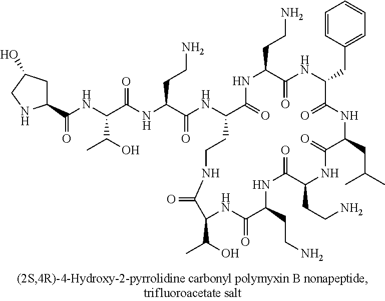 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@@H]2C[C@@H](O)CN2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxliv):

| Formula | Mass |
|---|---|
| C51H87N15O12 | 1101.67 |

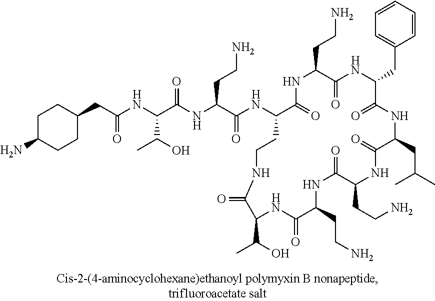 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)C[C@H]2CC[C@@H](N)CC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxlv):

| Formula | Mass |
|---|---|
| C49H83N15O12 | 1073.63 |

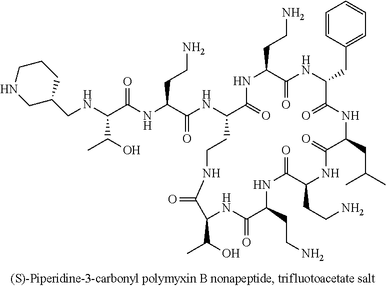 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC[C@H]2CCCNC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxlvi):

| Formula | Mass |
|---|---|
| C56H85N15O12 | 1159.65 |

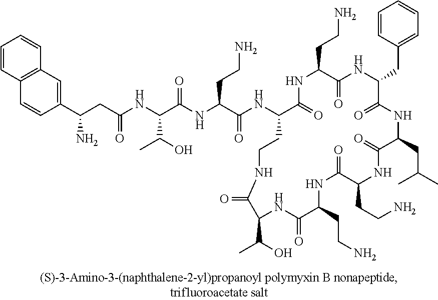 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)C[C@H](N)c2ccc3ccccc3c2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxlvii):

| Formula | Mass |
|---|---|
| C56H97N17O13 | 1215.75 |

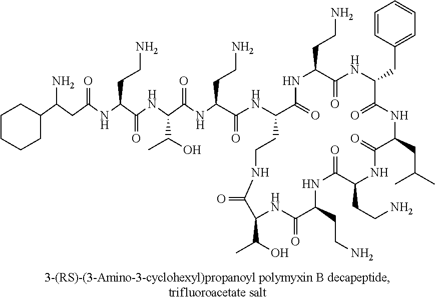 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@H](CCN)NC(=O)CC(N)C2CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxlviii):

| Formula | Mass |
|---|---|
| C51H87N15O12 | 1101.67 |

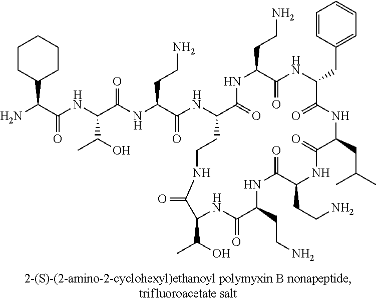 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@@H](N)C2CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (xxlix):

| Formula | Mass |
|---|---|
| C50H87N15O13 | 1105.66 |

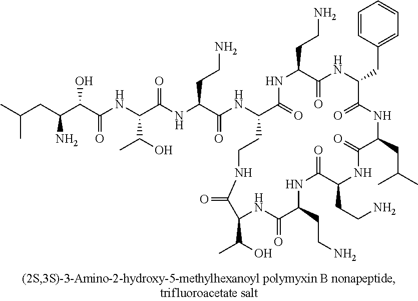 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@@H](O)[C@@H](N)CC(C)C)C(C)O)CCNC(=O)[C@H](C(C)O)NC(=O)[C@H](CCN)NC(=O)[C@H](CCN)NC1=O]

In some cases, a suitable polymyxin derivative is a compound of Formula (l):

| Formula | Mass |
|---|---|
| C51H87N15O12 | 1101.67 |

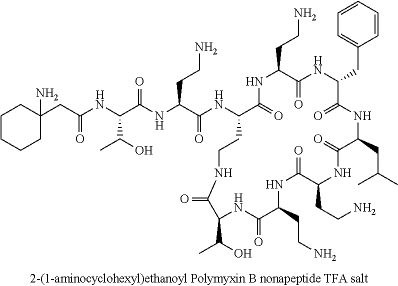 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CC2(N)CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (li):

| Formula | Mass |
|---|---|
| C48H81N15O12 | 1060.27 |

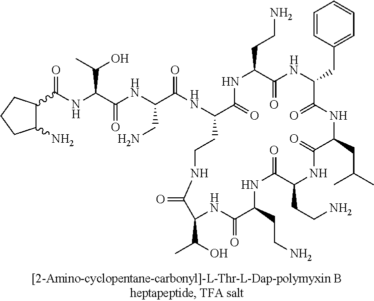 [SMILES: CC(C)C[C@@H]1NC(=O)[C@@H](Cc2ccccc2)NC(=O)[C@H](CCN)NC(=O)[C@@H](NC(=O)[C@H](CN)NC(=O)[C@@H](NC(=O)C2CCCC2N)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lii):

| Formula | Mass |
|---|---|
| C48H80N14O13 | 1061.26 |

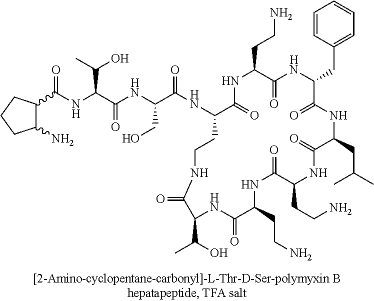 [SMILES: CC(C)C[C@@H]1NC(=O)[C@@H](Cc2ccccc2)NC(=O)[C@H](CCN)NC(=O)[C@@H](NC(=O)[C@H](CO)NC(=O)[C@@H](NC(=O)C2CCCC2N)C(C)O)CCNC(=O)[C@H](C(C)O)NC(=O)[C@H](CCN)NC(=O)[C@H](CCN)NC1=O]

In some cases, a suitable polymyxin derivative is a compound of Formula (liii):

| Formula | Mass |
|---|---|
| C50H84N14O13 | 1089.31 |

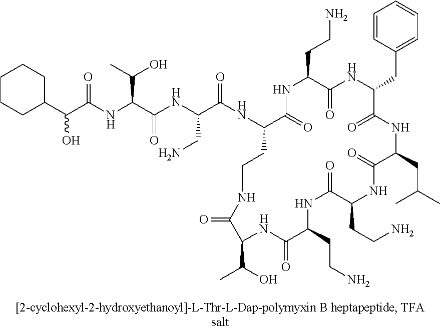 [SMILES: CC(C)C[C@@H]1NC(=O)[C@@H](Cc2ccccc2)NC(=O)[C@H](CCN)NC(=O)[C@@H](NC(=O)[C@H](CN)NC(=O)[C@@H](NC(=O)C(O)C2CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (liv):

| Formula | Mass |
|---|---|
| C50H83N13O14 | 1090.3 |

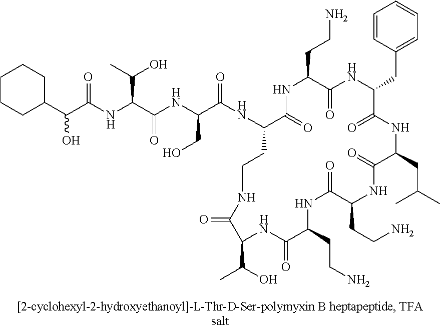 [SMILES: CC(C)C[C@@H]1NC(=O)[C@@H](Cc2ccccc2)NC(=O)[C@H](CCN)NC(=O)[C@@H](NC(=O)[C@@H](CO)NC(=O)[C@@H](NC(=O)C(O)C2CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (Iv):

| Formula | Mass |
|---|---|
| C51H87N15O12 | 1102.4 |

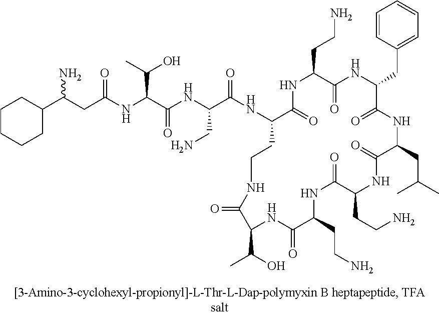 [SMILES: CC(C)C[C@@H]1NC(=O)[C@@H](Cc2ccccc2)NC(=O)[C@H](CCN)NC(=O)[C@@H](NC(=O)[C@H](CN)NC(=O)[C@@H](NC(=O)CC(N)C2CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lvi):

| Formula | Mass |
|---|---|
| C51H86N14O13 | 1103.3 |

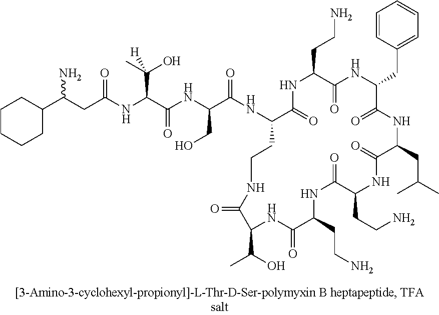 [SMILES: CC(C)C[C@@H]1NC(=O)[C@@H](Cc2ccccc2)NC(=O)[C@H](CCN)NC(=O)[C@@H](NC(=O)[C@@H](CO)NC(=O)[C@@H](NC(=O)CC(N)C2CCCCC2)[C@@H](C)O)CCNC(=O)[C@H](C(C)O)NC(=O)[C@H](CCN)NC(=O)[C@H](CCN)NC1=O]

In some cases, a suitable polymyxin derivative is a compound of Formula (lvii):

| Formula | Mass |
|---|---|
| C51H87N15O12 | 1102.4 |

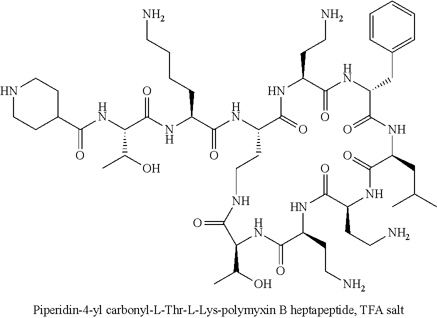 [SMILES: CC(C)C[C@@H]1NC(=O)[C@@H](Cc2ccccc2)NC(=O)[C@H](CCN)NC(=O)[C@@H](NC(=O)[C@H](CCCCN)NC(=O)[C@@H](NC(=O)C2CCNCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lviii):

| Formula | Mass |
|---|---|
| C49H84N16O12 | 1089.4 |

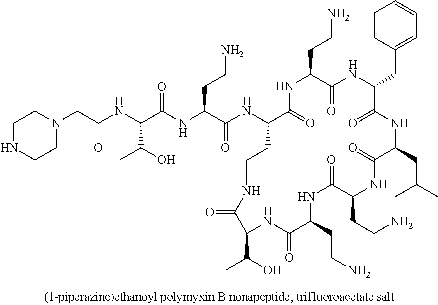 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CN2CCNCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lix):

| Formula | Mass |
|---|---|
| C53H91N17O13 | 1174.4 |

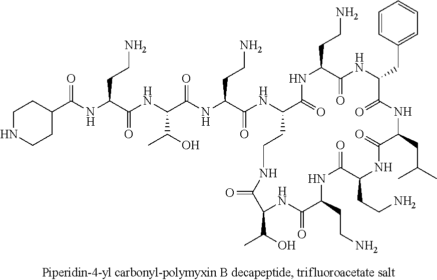 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@H](CCN)NC(=O)C2CCNCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lx):

| Formula | Mass |
|---|---|
| C55H95N17O13 | 1202.5 |

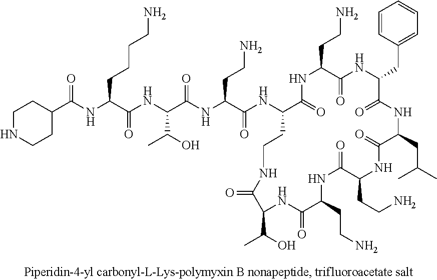 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@H](CCCCN)NC(=O)C2CCNCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lxi):

| Formula | Mass |
|---|---|
| C55H88N16O12 | 1164.7 |

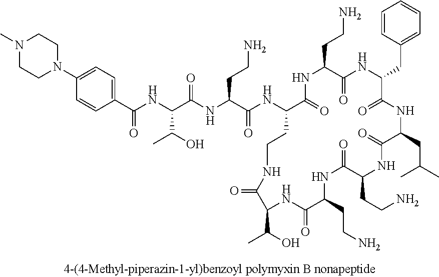 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)c2ccc(N3CCN(C)CC3)cc2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lxii):

| Formula | Mass |
|---|---|
| C50H85N13O13 | 1075.6 |

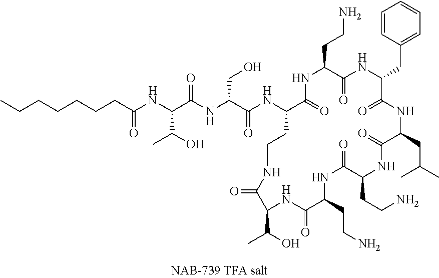 [SMILES: CCCCCCCC(=O)N[C@H](C(=O)N[C@H](CO)C(=O)N[C@H]1CCNC(=O)[C@H](C(C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)C(C)O]

In some cases, a suitable polymyxin derivative is a compound of Formula (lxiii):

| Formula | Mass |
|---|---|
| C54H86ClN17O13 | 1215.6 |

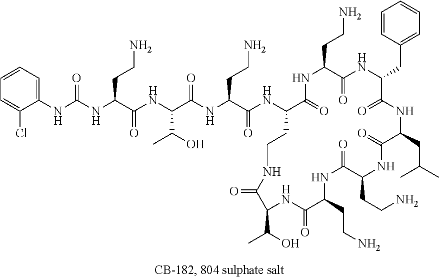 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@H](CCN)NC(=O)Nc2ccccc2Cl)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lxiv):

| Formula | Mass |
|---|---|
| C50H78N14O12 | 1066.6 |

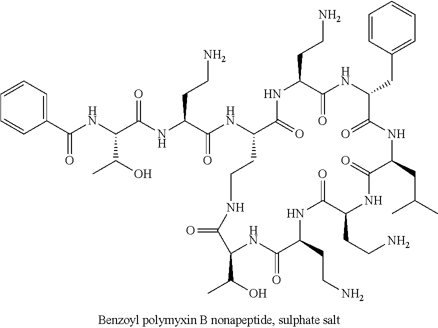 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)c2ccccc2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lxv):

| Formula | Mass |
|---|---|
| C51H86N14O12 | 1086.7 |

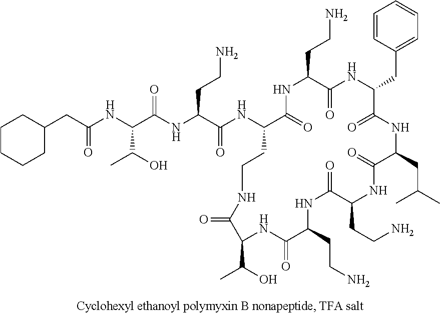 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)CC2CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lxv):

| Formula | Mass |
|---|---|
| C50H79N15O12 | 1081.6 |

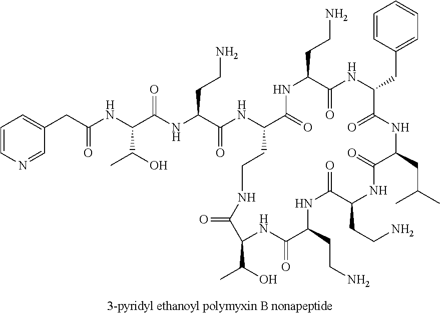 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)Cc2cccnc2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lxvi):

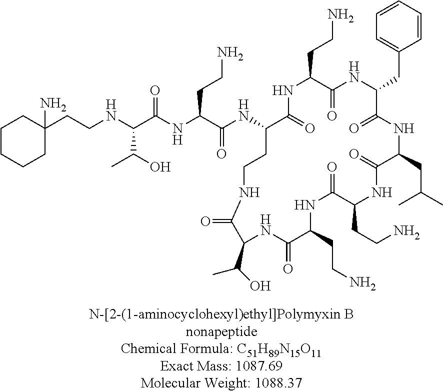 [SMILES: CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NCCC2(N)CCCCC2)C(C)O)CCNC(=O)[C@H](C(C)O)NC(=O)[C@H](CCN)NC(=O)[C@H](CCN)NC1=O]

In some cases, a suitable polymyxin derivative is a compound of Formula (lxvii):

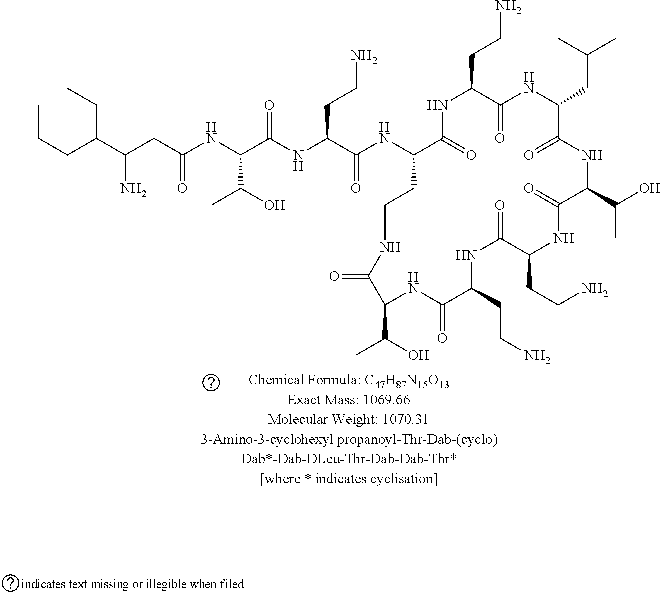 [SMILES: CCCC(CC)C(N)CC(=O)N[C@H](C(=O)N[C@@H](CCN)C(=O)N[C@H]1CCNC(=O)[C@H](C(C)O)NC(=O)[C@H](CCN)NC(=O)[C@H](CCN)NC(=O)[C@H](C(C)O)NC(=O)[C@@H](CC(C)C)NC(=O)[C@H](CCN)NC1=O)C(C)O]

In some cases, a suitable polymyxin derivative is a polymyxin E, Polymyxin B (PMB), C1 (NAB-739), or a C2 (CB-182,804) derivative. In some cases, CB-182, 804 (C2) is a polymyxin decapeptide derivative with an aryl urea substitute at the N-terminus.

In some cases, a suitable polymyxin derivative is a compound of Formula (lxviii):

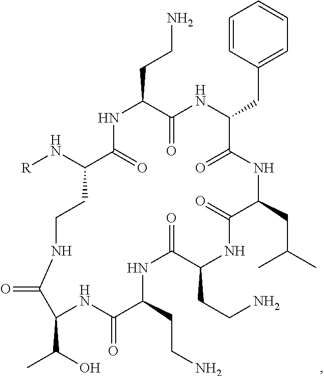

where Formula (lxviii) is a polymyxin B heptapeptide scaffold. The structures below depict the N-terminal group (—R) and side chain on the Polymyxin B heptapeptide scaffold of Formula (lxviii):

| | | | | Method | | |
|---|---|---|---|---|---|---|
| | | | | of | | |
| | | | | Prep- | | |
| Ex. | —R | Formula | Mass | aration | sm | Name |
| A1 | 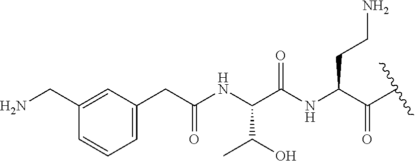 [SMILES: *C(=O)[C@H](CCN)NC(=O)[C@@H](NC(=O)Cc1cccc(CN)c1)C(C)O] | C52H83N15O12 | 1109.6 | 2 | Int 2 | 2-(3- (Aminomethyl) phenyl) ethanoyl polymyxin B nonapeptide |
| A2 | 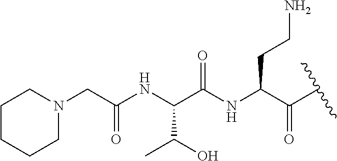 [SMILES: *C(=O)[C@H](CCN)NC(=O)[C@@H](NC(=O)CN1CCCCC1)C(C)O] | C50H85N15O12 | 1087.7 | 2 | Int. 2 | Piperidine-1- ylethanoyl polymyxin B nonapeptide |
| A3 | 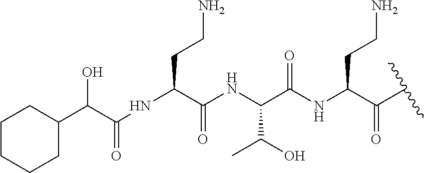 [SMILES: *C(=O)[C@H](CCN)NC(=O)[C@@H](NC(=O)[C@H](CCN)NC(=O)C(O)C1CCCCC1)C(C)O] | C55H94N16O14 | 1202.7 | 3B | Int. 7 | 2-(RS)-(2- Hydroxy-2- cyclohexyl) ethanoyl polymyxin B decapeptide |
| A4 | 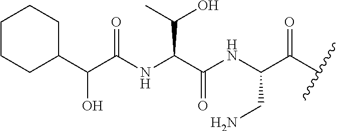 [SMILES: *C(=O)[C@H](CN)NC(=O)[C@@H](NC(=O)C(O)C1CCCCC1)C(C)O] | C50H84N14O13 | 1088.6 | 3A | Int. 5 | [2-(RS)-(2- Hydroxy-2- cyclohexyl) ethanoyl]-L- Thr-L-Dap- polymyxin B heptapeptide |
| A5 | 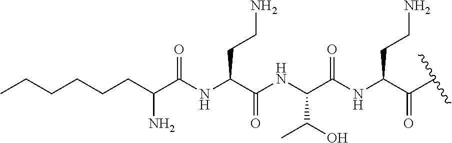 [SMILES: *C(=O)[C@H](CCN)NC(=O)[C@@H](NC(=O)[C@H](CCN)NC(=O)C(N)CCCCCC)C(C)O] | C55H97N17O13 | 1203.7 | 3B | Int. 7 | 2-(RS)-2- aminoethyl polymyxin B decapeptide |
| A6 | 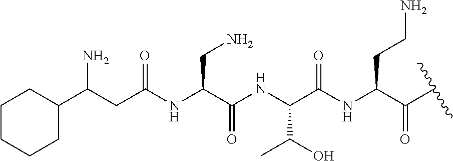 [SMILES: *C(=O)[C@H](CCN)NC(=O)[C@@H](NC(=O)[C@H](CN)NC(=O)CC(N)C1CCCCC1)C(C)O] | C52H95N17O13 | 1201.7 | 3B | Int. 7 | [3(R,S)-3- Amino-3- cyclohexane- propanoyl]- L-Dap- polymyxin B nonapeptide |
| A7 | 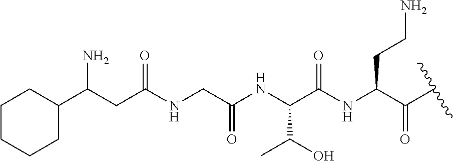 [SMILES: *C(=O)[C@H](CCN)NC(=O)[C@@H](NC(=O)CNC(=O)CC(N)C1CCCCC1)C(C)O] | C54H92N16O13 | 1172.7 | 3B | Int. 7 | [3(R,S)-3- Amino-3- cyclohexane- propanoyl]- Gly-polymyxin B nonapeptide |
| A8 | 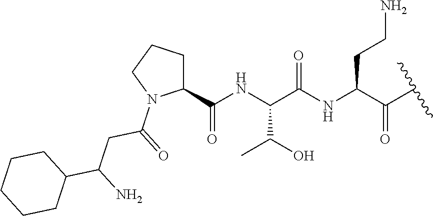 [SMILES: *C(=O)[C@H](CCN)NC(=O)[C@@H](NC(=O)[C@@H]1CCCN1C(=O)CC(N)C1CCCCC1)C(C)O] | C57H96N16O13 | 1212.7 | 3B | Int. 7 | [3(R,S)-3- Amino-3- cyclohexane- propanoyl]- L-Pro- polymyxin B nonapeptide isomer 1 |
| A9 | 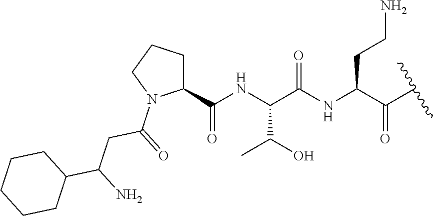 [SMILES: *C(=O)[C@H](CCN)NC(=O)[C@@H](NC(=O)[C@@H]1CCCN1C(=O)CC(N)C1CCCCC1)C(C)O] | C57H96N16O13 | 1212.7 | 3B | Int. 7 | [3(R,S)-3- Amino-3- cyclohexane- propanoyl]- L-Pro- polymyxin B nonapeptide isomer 2 |
| A10 | 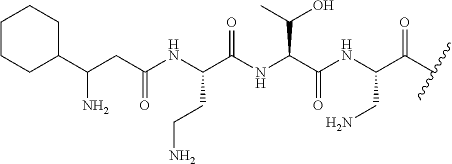 [SMILES: *C(=O)[C@H](CN)NC(=O)[C@@H](NC(=O)[C@H](CCN)NC(=O)CC(N)C1CCCCC1)C(C)O] | C55H95N17O13 | 1201.7 | 3A | Int. 5 | [3(R,S)-3- Amino-3- cyclohexane- propanoyl]- L-Dab-L- Thr-L-Dap polymyxi [symbol] B heptapeptide |
| A11 | 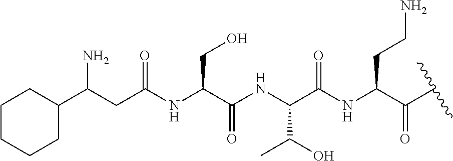 [SMILES: *C(=O)[C@H](CCN)NC(=O)[C@@H](NC(=O)[C@H](CO)NC(=O)CC(N)C1CCCCC1)C(C)O] | C55H94N16O14 | 1202.7 | 3B | Int. 7 | [3(R,S)-3- Amino-3- cyclohexane- propanoyl]- L-Ser- polymyxin B nonapeptide |
| A12 | 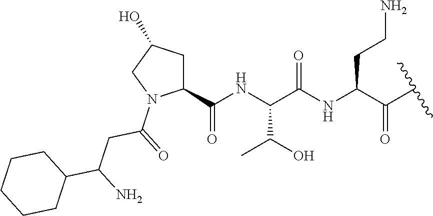 [SMILES: *C(=O)[C@H](CCN)NC(=O)[C@@H](NC(=O)[C@@H]1C[C@@H](O)CN1C(=O)CC(N)C1CCCCC1)C(C)O] | C57H96N16O14 | 1228.7 | 3B | Int. 7 | [3(R,S)-3- Amino-3- cyclohexane- propanoyl]- [4R-4- hydroxy-L-Pro]- polymyxin B nonapeptide |
| A13 | 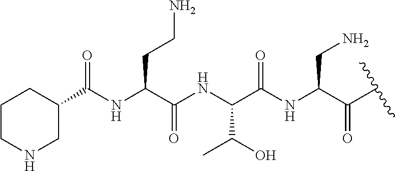 [SMILES: *C(=O)[C@H](CN)NC(=O)[C@@H](NC(=O)[C@H](CCN)NC(=O)[C@H]1CCCNC1)C(C)O] | C52H89N17O13 | 1139.7 | 3A | Int. 5 | [(3S)-piperidine- 3-carbonyl]- L-Dab-L- Thr-L-Dap polymyxin B heptapeptide |
| A14 | 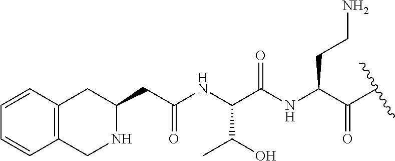 [SMILES: *C(=O)[C@H](CCN)NC(=O)[C@@H](NC(=O)C[C@@H]1Cc2ccccc2CN1)C(C)O] | C54H85N15O12 | 1135.7 | 2A | Int. 7 | (S)-2-((1,2,3,4)- tetrahydro- isoquinolin- 3-yl)ethanoyl polymyxin B nonapeptide |
| A15 | 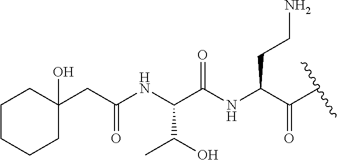 [SMILES: *C(=O)[C@H](CCN)NC(=O)[C@@H](NC(=O)CC1(O)CCCCC1)C(C)O] | C51H86N14O13 | 1102.6 | 2A | Int. 7 | 2-(1- Hydroxy- cyclohexyl) ethanoyl polymyxin B nonapeptide |
| A16 | 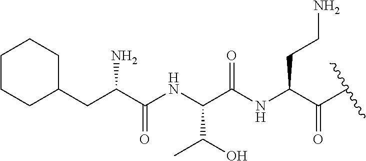 [SMILES: *C(=O)[C@H](CCN)NC(=O)[C@@H](NC(=O)[C@@H](N)CC1CCCCC1)C(C)O] | C52H89N15O12 | 1115.7 | 2A | Int. 7 | (2S)-2-Amino-3- cyclohexane- propanoyl polymyxin B nonapeptide |
| A17 | 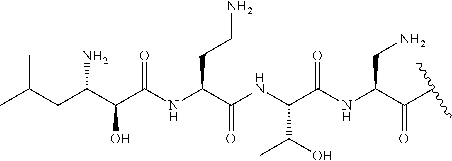 [SMILES: *C(=O)[C@H](CN)NC(=O)[C@@H](NC(=O)[C@H](CCN)NC(=O)[C@@H](O)[C@@H](N)CC(C)C)C(C)O] | C53H93N17O14 | 1191.7 | 2A | Int. 5 | [(2S,3S)-3- Amino- 2-hydroxy-5- methylhexanoyl]- L-Dab-L- Thr-L-Dap polymyxin B heptapeptide |
| A18 | 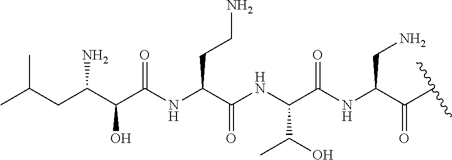 [SMILES: *C(=O)[C@H](CN)NC(=O)[C@@H](NC(=O)[C@H](CCN)NC(=O)[C@@H](O)[C@@H](N)CC(C)C)C(C)O] | C54H95N17O14 | 1205.7 | 2A | Int. 8 | (2S,3S)-3-Amino- 2-hydroxy-5- methylhexanoyl polymyxin B decapeptide |
| A19 | 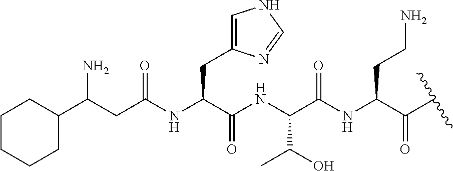 [SMILES: *C(=O)[C@H](CCN)NC(=O)[C@@H](NC(=O)[C@H](Cc1c[nH]cn1)NC(=O)CC(N)C1CCCCC1)C(C)O] | C58H96N18O13 | 1252.7 | 3B | Int. 7 | [3(R,S)-3-Amino- 3-cyclohexane- propanoyl]- L-His-polymyxin B nonapeptide |
| A20 | 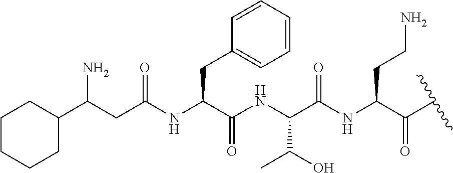 [SMILES: *C(=O)[C@H](CCN)NC(=O)[C@@H](NC(=O)[C@H](Cc1ccccc1)NC(=O)CC(N)C1CCCCC1)C(C)O] | C61H98N16O13 | 1262.7 | 3B | Int. 7 | [3(R,S)-3-Amino- 3-cyclohexane- propanoyl]- L-Phe-polymyxin B nonapeptide isomer 1 |
| A21 | 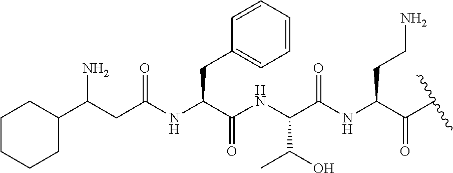 [SMILES: *C(=O)[C@H](CCN)NC(=O)[C@@H](NC(=O)[C@H](Cc1ccccc1)NC(=O)CC(N)C1CCCCC1)C(C)O] | C61H98N16O13 | 1262.7 | 3B | Int. 7 | [3(R,S)-3-Amino- 3-cyclohexane- propanoyl]- L-Phe-polymyxin B nonapeptide isomer 2 |
| A22 | 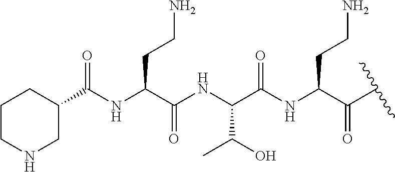 [SMILES: *C(=O)[C@H](CCN)NC(=O)[C@@H](NC(=O)[C@H](CCN)NC(=O)[C@H]1CCCNC1)C(C)O] | C53H9 [symbol] N17O13 | 1173.7 | 2A | Int. 8 | (S)-Piperidine-3- carbonyl polymyxin B decapeptide, |
| A23 | 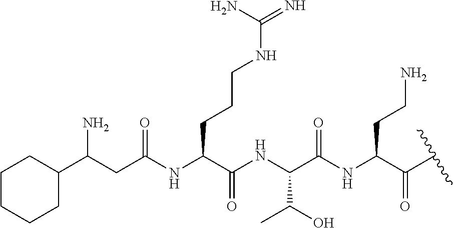 [SMILES: *C(=O)[C@H](CCN)NC(=O)[C@@H](NC(=O)[C@H](CCCNC(=N)N)NC(=O)CC(N)C1CCCCC1)C(C)O] | C58H101N19O13 | 1271.8 | 3B | Int. 7 | [3(R,S)-3- Amino-3- cyclohexane- propanoyl]- L-Arg- polymyxin B nonapeptide |
| A24 | 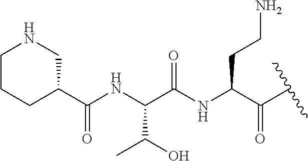 [SMILES: *C(=O)[C@H](CCN)NC(=O)[C@@H](NC(=O)[C@@H]1CCCNC1)C(C)O] | C49H83N15O12 | 1073.6 | 2A | Int. 7 | (R)-Piperidine-3- carbonyl polymyxin B nonapeptide |
| A25 | 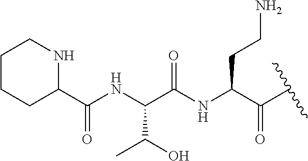 [SMILES: *C(=O)[C@H](CCN)NC(=O)[C@@H](NC(=O)C1CCCCN1)C(C)O] | C49H83N15O12 | 1073.6 | 2A | Int. 7 | Piperidine-2- carbonyl polymyxin B nonapeptide. Isomer 1 |
| A26 | 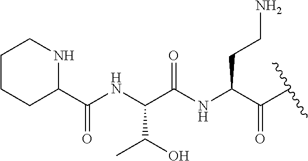 [SMILES: *C(=O)[C@H](CCN)NC(=O)[C@@H](NC(=O)C1CCCCN1)C(C)O] | C49H83N15O12 | 1073.6 | 2A | Int. 7 | Piperidine-2- carbonyl polymyxin B nonapeptide. Isomer 2 |
| A27 | 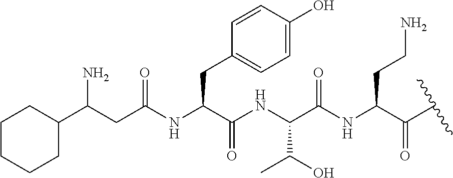 [SMILES: *C(=O)[C@H](CCN)NC(=O)[C@@H](NC(=O)[C@H](Cc1ccc(O)cc1)NC(=O)CC(N)C1CCCCC1)C(C)O] | C61H98N16O14 | 1278.7 | 3B | Int. 7 | [3(R,S)-3- Amino-3- cyclohexane- propanoyl]- L-Tyr- polymyxin B nonapeptide |
| A28 | 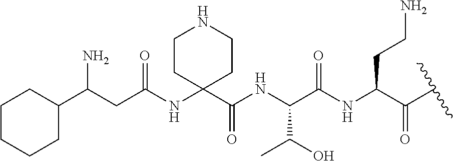 [SMILES: *C(=O)[C@H](CCN)NC(=O)[C@@H](NC(=O)C1(NC(=O)CC(N)C2CCCCC2)CCNCC1)C(C)O] | C58H99N17O13 | 1241.8 | 3B | Int. 7 | 4-[3(R,S)-3- Amino-3- cyclohexane- propanamido] piperidine-4- carbonyl polymyxin B nonapeptide |
| A29 | 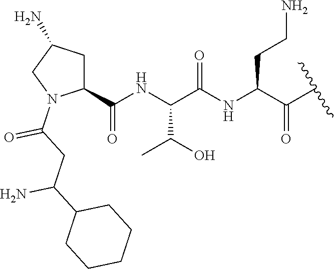 [SMILES: *C(=O)[C@H](CCN)NC(=O)[C@@H](NC(=O)[C@@H]1C[C@@H](N)CN1C(=O)CC(N)C1CCCCC1)C(C)O] | C57H97N17O13 | 1227.7 | 3B | Int. 7 | (2S,4R)-4-amino- 1-(3-amino- 3-cyclohexyl- propanoyl) pyrroli [symbol] 2-carbonyl polymyxin B nonapeptide |
| A30 | 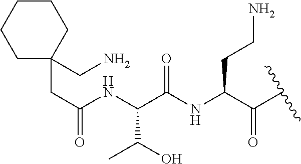 [SMILES: *C(=O)[C@H](CCN)NC(=O)[C@@H](NC(=O)CC1(CN)CCCCC1)C(C)O] | C52H89N15O12 | 1115.7 | 2A | Int. 7 | 2-(1- (Aminomethyl) cyclohexyl) ethanoyl Polymyxin B nonapeptide |
| A31 | 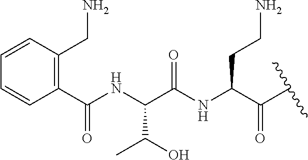 [SMILES: *C(=O)[C@H](CCN)NC(=O)[C@@H](NC(=O)c1ccccc1CN)C(C)O] | C51H81N15O12 | 1095.6 | 2A | Int. 7 | 2-Amino- methylbenzoyl polymyxin B nonapeptide |
| A32 | 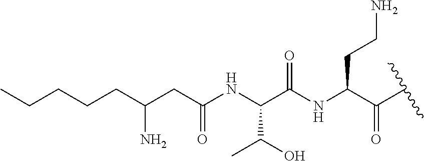 [SMILES: *C(=O)[C@H](CCN)NC(=O)[C@@H](NC(=O)CC(N)CCCCC)C(C)O] | C51H89N15O12 | 1103.7 | 2A | Int. 7 | 3-(RS)-3-amino octanoyl polymyxin B nonapeptide. Isomer 1 |
| A33 | 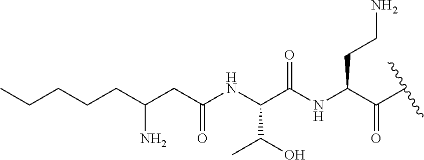 [SMILES: *C(=O)[C@H](CCN)NC(=O)[C@@H](NC(=O)CC(N)CCCCC)C(C)O] | C51H89N15O12 | 1103.7 | 2A | Int. 7 | 3-(RS)-3-amino octanoyl polymyxin B nonapeptide. Isomer 2 |
| A34 | 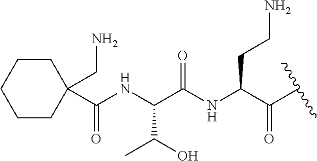 [SMILES: *C(=O)[C@H](CCN)NC(=O)[C@@H](NC(=O)C1(CN)CCCCC1)C(C)O] | C51H87N15O12 | 1101.7 | 2A | Int. 7 | (1- aminomethyl- cyclohexane) carbonyl polymyxin B nonapeptide |
| A35 | 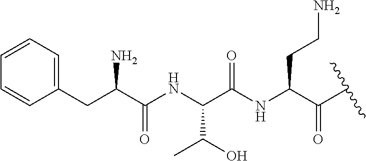 [SMILES: *C(=O)[C@H](CCN)NC(=O)[C@@H](NC(=O)[C@H](N)Cc1ccccc1)C(C)O] | C52H83N15O12 | 1109.6 | 2A | Int 7 | D-Phe polymyxin B nonapeptide |
| A36 | 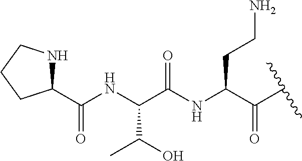 [SMILES: *C(=O)[C@H](CCN)NC(=O)[C@@H](NC(=O)[C@H]1CCCN1)C(C)O] | C48H81N15O12 | 1059.6 | 2A | Int. 7 | D-Pro polymyxin B nonapeptide |
| A37 | 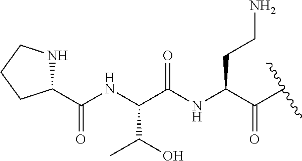 [SMILES: *C(=O)[C@H](CCN)NC(=O)[C@@H](NC(=O)[C@@H]1CCCN1)C(C)O] | C48H81N15O12 | 1059.6 | 2A | Int. 7 | L-Pro polymyxin B nonapeptide |
| A38 | 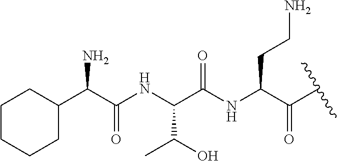 [SMILES: *C(=O)[C@H](CCN)NC(=O)[C@@H](NC(=O)[C@H](N)C1CCCCC1)C(C)O] | C51H87N15O12 | 1101.7 | 2A | Int. 7 | 2-(R)-(2-amino- 2-cyclohexyl) ethanoyl) polymyxin B nonapeptide |
| A39 | 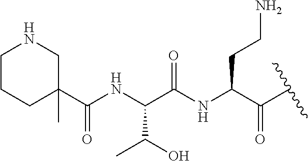 [SMILES: *C(=O)[C@H](CCN)NC(=O)[C@@H](NC(=O)C1(C)CCCNC1)C(C)O] | C50H85N15O12 | 1087.7 | 2A | Int. 7 | 3-Methyl- piperidine- 3-carbonyl polymyxin B nonapeptide |
| A40 | 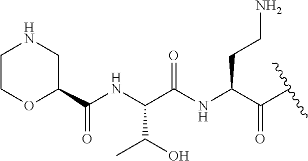 [SMILES: *C(=O)[C@H](CCN)NC(=O)[C@@H](NC(=O)[C@@H]1CNCCO1)C(C)O] | C48H81N15O13 | 1075.6 | 2A | Int. 7 | (2S)-Morpholine- 2-carbonyl polymyxin B nonapeptide |
| A42 | 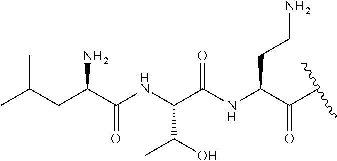 [SMILES: *C(=O)[C@H](CCN)NC(=O)[C@@H](NC(=O)[C@H](N)CC(C)C)C(C)O] | C49H85N15O12 | 1075.7 | 2A | Int. 7 | D-Leu-polymyxin B nonapeptide |
| A43 | 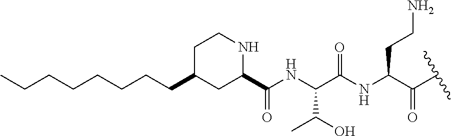 | C57H99N15O12 | 1185.8 | 2A | Int. 7 | Cis-4-Octyl piperidine- 2-carbonyl polymyxin B nonapeptide. Isomer 1 |
| A44 | 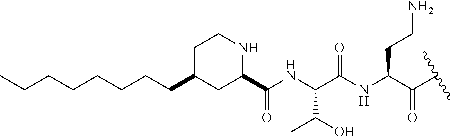 | C57H99N15O12 | 1185.8 | 2A | Int. 7 | Cis-4-Octyl piperidine- 2-carbonyl polymyxin B nonapeptide. Isomer 2 |
| A45 | 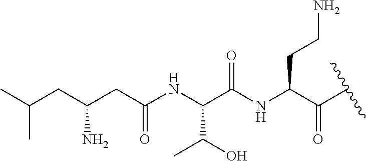 [SMILES: *C(=O)[C@H](CCN)NC(=O)[C@@H](NC(=O)C[C@H](N)CC(C)C)C(C)O] | C50H87N15O12 | 1089.7 | 2A | Int. 7 | 3-(R)-3-Amino-5- methylhexanoyl polymyxin B nonapeptide |
| A46 | 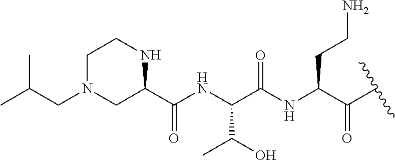 [SMILES: *C(=O)[C@H](CCN)NC(=O)[C@@H](NC(=O)[C@H]1CN(CC(C)C)CCN1)C(C)O] | C52H90N16O12 | 1130.7 | 2A | Int. 7 | (R)-4-isobutyl- piperazine-2- carbonyl polymyxin B nonapeptide |
| A47 | 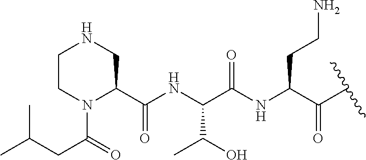 [SMILES: *C(=O)[C@H](CCN)NC(=O)[C@@H](NC(=O)[C@@H]1CNCCN1C(=O)CC(C)C)C(C)O] | C53H90N16O13 | 1158.7 | 2A | Int. 7 | (S)-1-(3- methylbutanoyl) piperazine-2- carbonyl polymyxin B nonapeptide. |
| A48 | 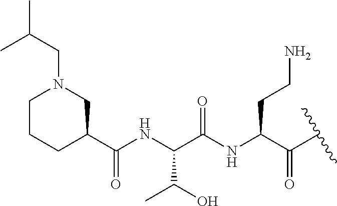 [SMILES: *C(=O)[C@H](CCN)NC(=O)[C@@H](NC(=O)[C@H]1CCCN(CC(C)C)C1)C(C)O] | C53H91N15O12 | 1129.69 7048 | 2A | Int. 7 | (S)-1-(2- methylpropyl)- piperidine-3- carbonyl polymyxin B nonapeptide |
| A49 | 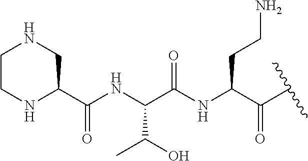 [SMILES: *C(=O)[C@H](CCN)NC(=O)[C@@H](NC(=O)[C@@H]1CNCCN1)C(C)O] | C48H82N16O12 | 1074.62 9692 | 2A | Int. 7 | (2S)-piperazine- 2-carbonyl polymyxin B nonapeptide |
| A50 | 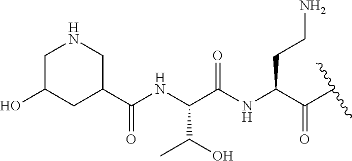 [SMILES: *C(=O)[C@H](CCN)NC(=O)[C@@H](NC(=O)C1CNCC(O)C1)C(C)O] | C49H83N15O13 | 1089.6 | 2A | Int. 7 | 5-Hydroxy- piperidine- 3-carbonyl polymyxin B nonapeptide |
| C6 | 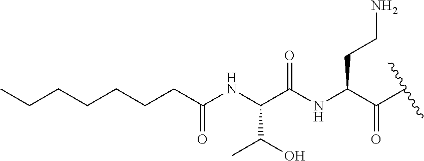 [SMILES: *C(=O)[C@H](CCN)NC(=O)[C@@H](NC(=O)CCCCCCC)C(C)O] | C51H88N14O12 | 1088.7 | 1 | Int 2 | Octanoyl polymyxin B nonapeptide, sulfate salt. |

[symbol]

indicates data missing or illegible when filed

In some cases, the polypeptide that enhances antimicrobial activity of an antibiotic is polymyxin antibiotic. In some cases, the polymyxin antibiotic is a polymyxin derivative. In some cases, a suitable polymyxin derivative is a polymyxin compound as described in International Patent Application Publication No.: WO 2009/098357, which is hereby incorporated by reference in its entirety.

In some cases, suitable polymyxin derivatives are found in Zabawa et al. 2016 (Zabawa et al. (2016) Current Opinion in Microbiology 33: 7-12). Suitable polymyxin derivatives found in Zabawa et al. are disclosed in Formulas (lxix-lxxi) shown below.

In some cases, a suitable polymyxin derivative is a derivative compound of Formula (lxix):

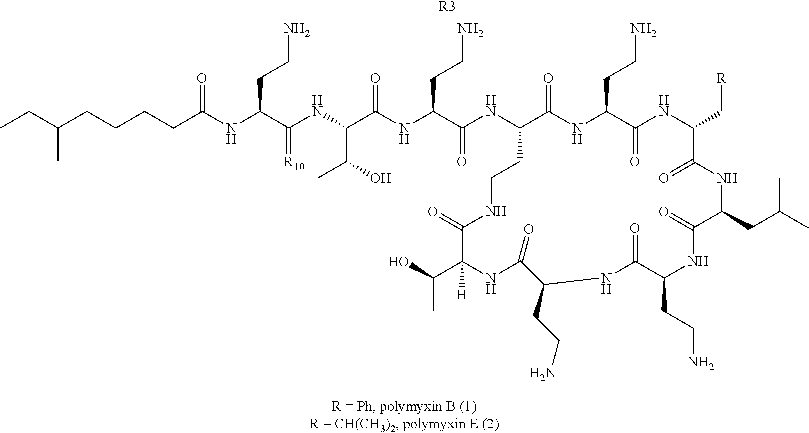

In some cases, a suitable polymyxin derivative is a derivative compound of Formula (lxx):

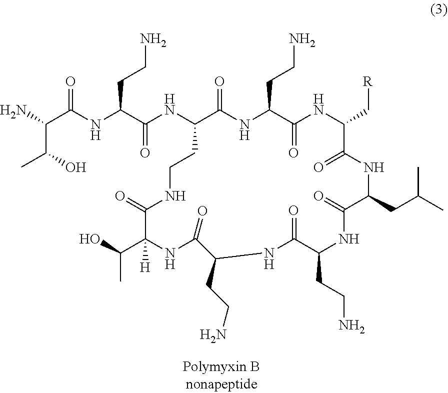

In some cases, a suitable polymyxin derivative is a compound of Formula (lxxi):

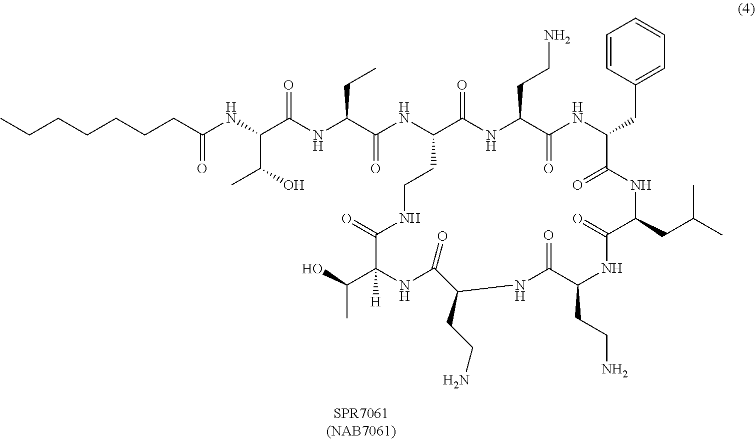 [SMILES: CCCCCCCC(=O)N[C@H](C(=O)N[C@@H](CC)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

In some cases, a suitable polymyxin derivative is a compound of Formula (lxxi):

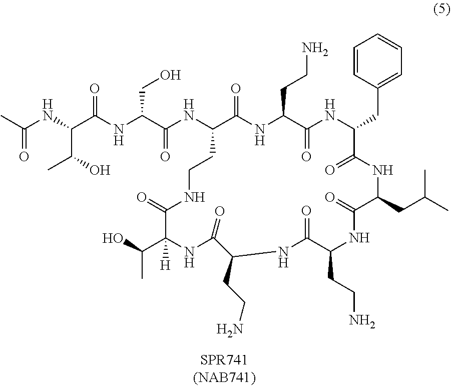 [SMILES: CC(=O)N[C@H](C(=O)N[C@H](CO)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

In some cases, suitable polymyxin derivatives are found in U.S. Patent Application Publication No.: 20130345121, which is hereby incorporated by reference in its entirety.

In some cases, a suitable polymyxin derivative is a compound of Formula (lxxii):

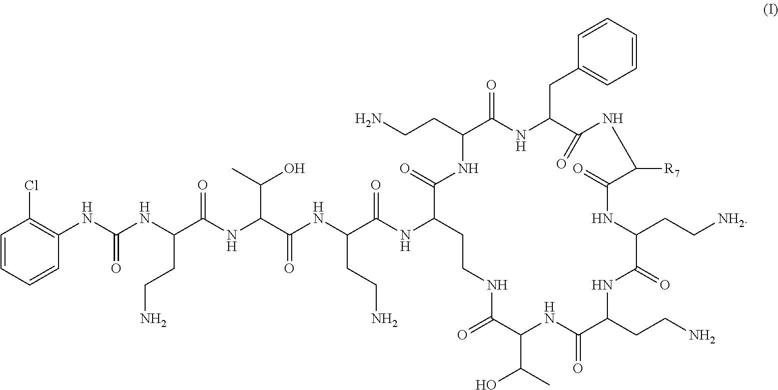

In some cases, a suitable polymyxin derivative is a compound of Formula (lxxiii):

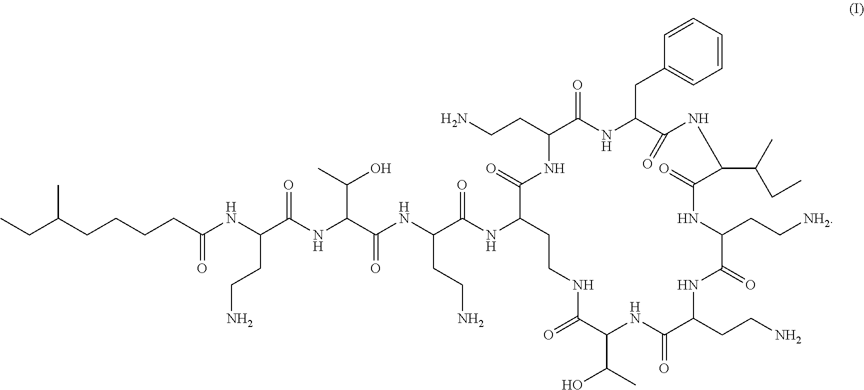 [SMILES: CCC(C)CCCCC(=O)NC(CCN)C(=O)NC(C(=O)NC(CCN)C(=O)NC1CCNC(=O)C(C(C)O)NC(=O)C(CCN)NC(=O)C(CCN)NC(=O)C(C(C)CC)NC(=O)C(Cc2ccccc2)NC(=O)C(CCN)NC1=O)C(C)O]

In some cases, a suitable polymyxin derivative is a compound of Formula (lxxiv):

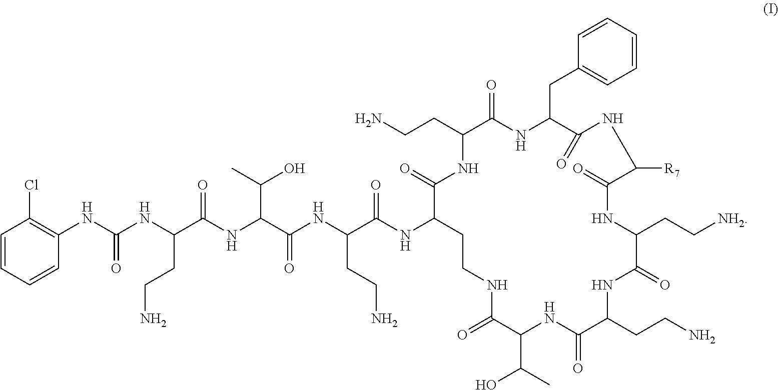

where:

R₇ is an alkyl moiety selected from isobutyl and sec-butyl, as well as pharmaceutically acceptable derivatives or pharmaceutically acceptable salts thereof.

For example, pharmaceutical compositions with antibacterial activity can include one or more compounds of Formula (lxxii), such as a compound of Formula (Ia), a compound of Formula (Ib), specific enantiomers of Formula (Ia) or Formula (Ib), or any combination thereof, or pharmaceutically acceptable salts (e.g., a sulfate salt) or derivatives (e.g., esters) thereof:

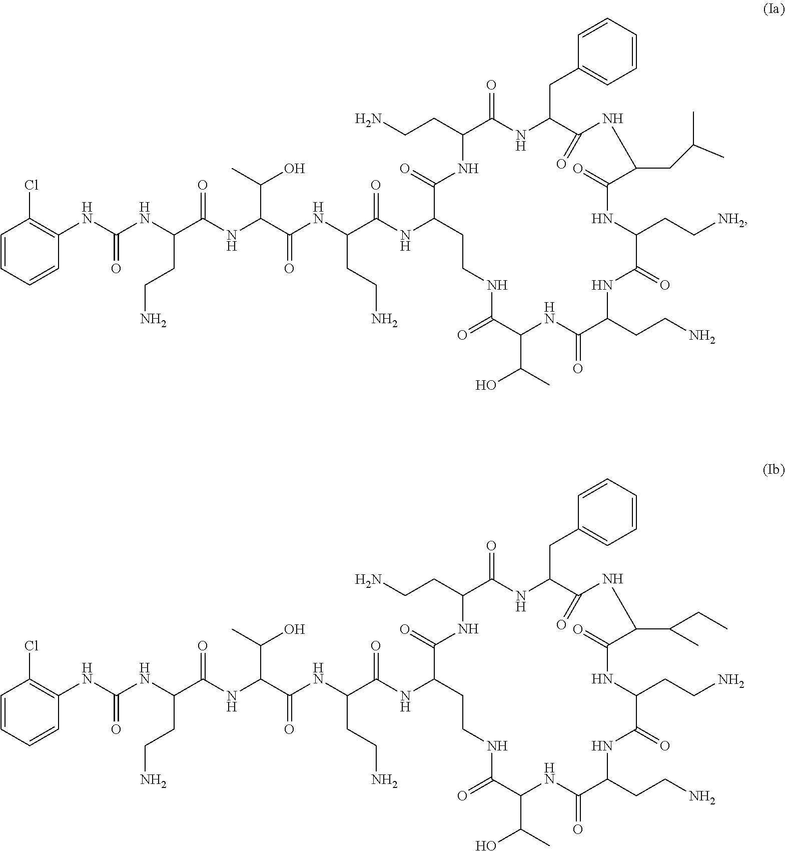 [SMILES: CC(C)CC1NC(=O)C(Cc2ccccc2)NC(=O)C(CCN)NC(=O)C(NC(=O)C(CCN)NC(=O)C(NC(=O)C(CCN)NC(=O)Nc2ccccc2Cl)C(C)O)CCNC(=O)C(C(C)O)NC(=O)C(CCN)NC(=O)C(CCN)NC1=O ; CCC(C)C1NC(=O)C(Cc2ccccc2)NC(=O)C(CCN)NC(=O)C(NC(=O)C(CCN)NC(=O)C(NC(=O)C(CCN)NC(=O)Nc2ccccc2Cl)C(C)O)CCNC(=O)C(C(C)O)NC(=O)C(CCN)NC(=O)C(CCN)NC1=O]

One particular example of an antibacterial compound of Formula (Ia) is described by Formula (Ic) and formula Ic′) below:

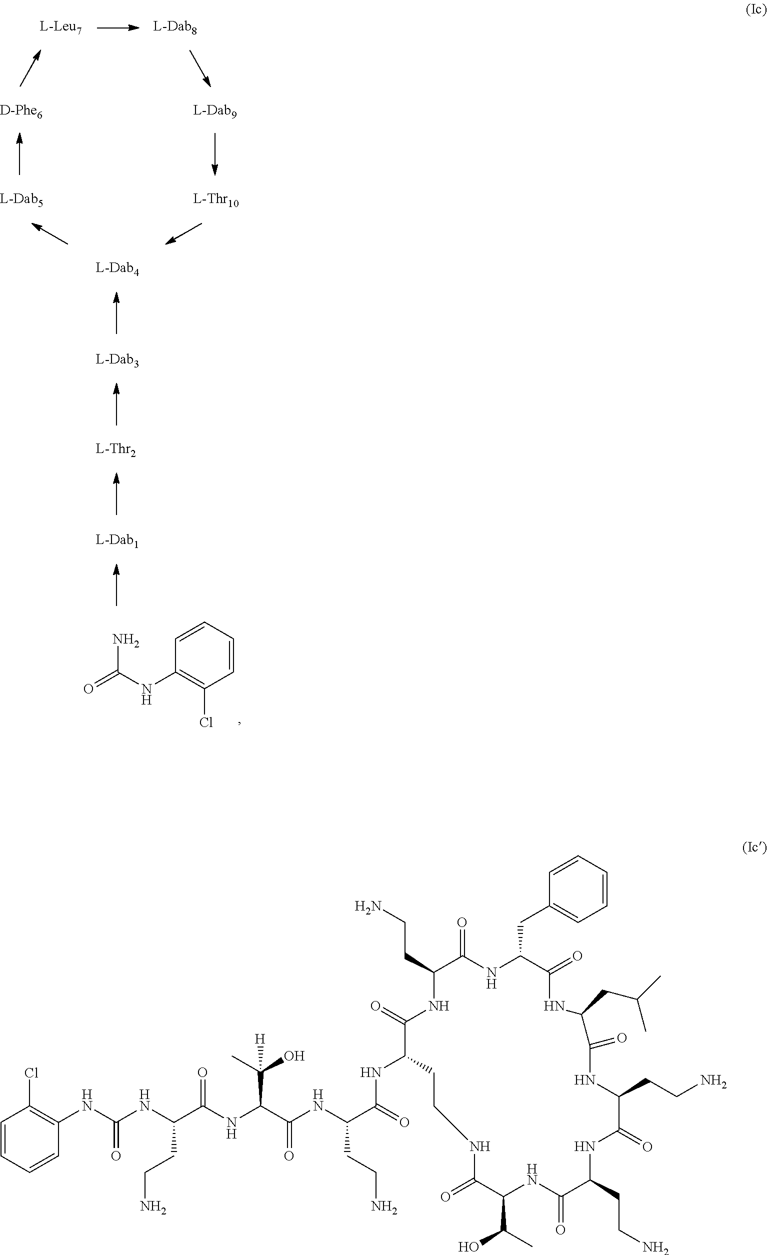 [SMILES: NC(=O)Nc1ccccc1Cl ; CC(C)C[C@@H]1NC(=O)[C@@H](Cc2ccccc2)NC(=O)[C@H](CCN)NC(=O)[C@@H](NC(=O)[C@H](CCN)NC(=O)[C@@H](NC(=O)[C@H](CCN)NC(=O)Nc2ccccc2Cl)[C@@H](C)O)CCNC(=O)[C@H]([C@@H](C)O)NC(=O)[C@H](CCN)NC(=O)[C@H](CCN)NC1=O]

In some cases, a suitable polymyxin derivative is a compound of Formula (LXXV):

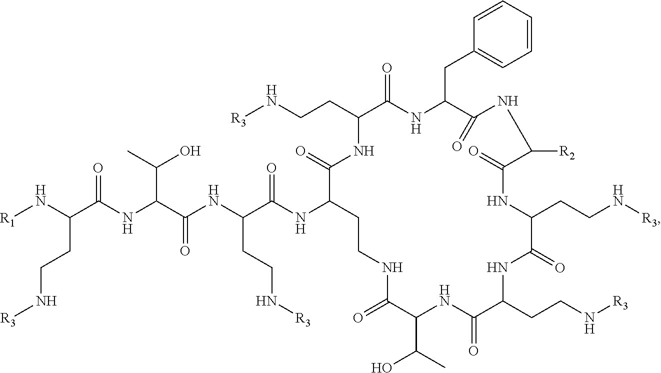

where:

R₁ is chosen from H, and —C(O)NHR_{A}, wherein R_{A} is chosen from benzyl and phenyl, and wherein both said benzyl and phenyl may be optionally substituted with one or more halo and/or nitro;

R₂ is chosen front —CH₂CH(CH₃)₂ and —C(CH₃)CH₂CH₃; and

R₃ is H.

In some cases, suitable polymyxin derivatives may be found in, for example, U.S. Patent Application Publication No.: 2013/0345121, which is hereby incorporated by reference in its entirety.

Aminoglycosides

In some cases, the antimicrobial agent is an aminoglycoside antibiotic. Aminoglycoside antibiotics function by binding to the A-site decoding region of bacterial rRNA causing mistranslation and/or premature message termination. Aminoglycosides are a group of bactericidal drugs sharing chemical, antimicrobial, pharmacologic, and toxic characteristics. The group includes streptomycin, neomycin, kanamycin, amikacin, gentamycin, tobramycin, sisomicin, arbekacin, netilmicin, paromomycin, and spectinomycin. Formulations of Aminoglycosides can be found in, for example, U.S. Patent Publication Nos.: 9486462 and 7244712, which are hereby incorporated by reference in their entirety.

In some cases, the aminoglycoside antibiotic is selected from the group consisting of streptomycin, neomycin, kanamycin, amikacin, gentamycin, tobramycin, sisomicin, arbekacin, apramycin, netilmicin, paromomycin, and spectinomycin. In some cases, the aminoglycoside antibiotic is selected from the group consisting of amikacin chloride, tobramycin sulfate, gentamycin sulfate, and gentamycin chloride. In some cases, the aminoglycoside antibiotic is an L-aminoglycoside antibiotic. L-aminoglycoside compounds are selected from L-neamine, L-neamine diasteromers differing from L-neamine in the stereochemical identity of between 1 and 3 stereocenters, and aminoglycosides having a L-neamine or L-neamine diastereomer coupled to one or more D- or L-sugar or D- or L-azasugar residues. In some cases, aminoglycosides may be optionally substituted at one or more hydroxyl or amino functional groups.

Aminoglycosides inhibit protein synthesis in bacteria by inhibiting the protein synthesis function of the bacterial ribosome. All aminoglycosides are potentially ototoxic (damage to the ear) and nephrotoxic (damage to the kidneys). Because of their toxicity and the availability of less toxic antibiotics, aminoglycosides have been used less often in recent years and to treat resistant Gram-negative organisms that are sensitive only to aminoglycosides. Combinations of tobramycin with fosfomycin are described in Baker et al. U.S. Pat. No. 7,943,118.

Amikacin is a synthetic aminoglycoside used to manage infections caused by Gram-negative bacilli resistant to gentamycin and tobramycin. Amikacin is most commonly used on serious Gram-negative infections involving skin and soft tissue, bone and joint, abdominal and urinary tract, and severe respiratory infections. Amikacin's use can include coverage against some aerobic Gram-positive bacteria, which include E. coli, klebsiella, proteus, pseudomonas, salmonella, enterobacter, serratia and mycoplasma. Like other aminoglycosides, amikacin has a similar potential for ototoxicity and nephrotoxicity especially when given by parenteral administration due to systemic absorption. Amikacin used for intravenous administration is formulated as amikacin-sulfate. Aminoglycosides specially formulated for stability and safety, include amikacin chloride, tobramycin sulfate, and gentamycin sulfate or chloride, in a combination composition with fosfomycin solutions.

### Quinolones

In some cases, the antimicrobial agent is a quinolone antibiotic. In some cases, quinolone antibiotics can be selected from the group consisting: of ciprofloxacin, levofloxacin, lomefloxacin, moxifloxacin, norfloxacin, ofloxacin, sparfloxacin, trvafloxacin, gatifloxacin, gemifloxacin, cinoxacin, and nalidixic acid. In some cases, the quinolone antibiotic is a fluoroquinolone antibiotic. In some cases, the fluoroquinolone antibiotic is selected from the group consisting of: ciprofloxacin, levofloxacin, gatifloxacin, moxifloxacin, ofloxacin, and norfloxacin. In some cases, the fluoroquinolone antibiotic is a ciprofloxacin antibiotic. Formulations for topical fluoroquinolone antibiotics can be found in, for example, U.S. Patent Publication No.: 5912255, which is hereby incorporated by reference in its entirety.

Fluoroquinolone antibiotics were first developed in the early 1960s but the earliest one, nalidixic acid, proved particularly susceptible to resistant bacteria thereby making it ineffectual over the long term. Fluoroquinolones attack bacteria by targeting DNA gyrase and by interfering with bacterial replication.

Theses antibiotics have been used to treat respiratory tract infections, urinary tract infections, diarrhea, postoperative-wound infections, and many other conditions, because they are readily absorbed after oral and topical administration and exhibit potent in vitro activity against a broad spectrum of bacterial species. U.S. Pat. No. 5,476,854 describes the oral, intravenous and transdermal use of lomefloxacin to treat urinary tract infections, upper respiratory tract infections, sexually-transmitted infections, opthalmological infections and intestinal infections.

In some cases, quinolones are hepatotoxic. In some cases, fluoroquinolones are hepatotoxic. In some cases, fluoroquinolones cause idiosyncratic liver injury. In some cases, the fluoroquinolones that are hepatotoxic include temafloxacin, gatifloxacin, and trovafloxacin.

Fluoroquinolone antibiotics are active against a wide spectrum of gram-positive and gram-negative bacteria because of their broad antimicrobial activity. Varieties of fluoroquinolones, specifically ciprofloxacin, have been found to be effective against Staphylococcus aureus, Streptococcus pneumoniae, coagulese-negative staphylococci, Streptococcus pyogenes, Staphylococcus epidermis, Pseudomonas aeruginosa, Escherichia coli, Klebsiella pneumoniae, Enterobacter cloacae, Proteus mirabilis, Proteus vulgaris, Providencia stuartii, Morganella morganii, Citrobacter diversus, Citrobacter freundii, and other susceptible organisms. The mounting resistance of Staphylococcus aureus to both penicillin and erythromycin has made the fluoroquinolone antibiotics a viable alternative for the treatment of skin diseases.

In some cases, the antimicrobial agent is a ciprofloxacin antibiotic.Ciprofloxacin is a fluoroquinolone antibiotic that is indicated for the treatment of lower respiratory tract infections due to P. aeruginosa, which is common in patients with cystic fibrosis. Ciprofloxacin is broad spectrum and, in addition to P. aeruginosa, is active against several other types of gram-negative and gram-positive bacteria. It acts by inhibition of topoisomerase II (DNA gyrase) and topoisomerase IV, which are enzymes required for bacterial replication, transcription, repair, and recombination. This mechanism of action is different from that for penicillins, cephalosporins, aminoglycosides, macrolides, and tetracyclines, and therefore bacteria resistant to these classes of drugs may be susceptible to ciprofloxacin. Formulations for ciprofloxacin can be found in, for example, U.S. Patent Publication No.: 9545401, which is hereby incorporated by reference in its entirety.

### Dibasic Macrolide

In some cases, the antimicrobial agent is a dibasic macrolide antibiotic. In some cases, the macrolide antibiotic is selected from azithromycin, clarithromycin, dirithromycin, erythromycin, roxithromycin, troleandomycin, telithromycin, and spectinomycin, or the like.

Macrolides are multi-membered lactone rings having one or more deoxy sugars as substituents. One group of macrolides is the clarithromycin class of compounds, where the ring structure was stabilized by the methylation of the C-6 hydroxyl. Another group of macrolides is the 15-membered ring aza analogs, such as azithromycin. Another group of macrolides is the 3-desglycosyl-3-oxo analogs, also known as ketolides, such as telithromycin and cethromycin.

In some cases, the dibasic macrolide is a dibasic erythromycin. In some cases, the dibasic macrolide is a dibasic clarithromycin. Erythromycin and clarithromycin are well known macrolides. Other erythromycin-based macrolide compounds have been prepared, e.g., by introducing modifications at various positions of erythromycin or clarithromycin, e.g., as in: U.S. Pat. Nos. 4,331,803; 4,474,768; 4,517,359; 5,523,399; 5,527,780; 5,635,485; 5,804,565; 6,020,521; 6,025,350; 6,075,133; 6,162,794; 6,191,118; 6,248,719; 6,291,656; 6,437,151; 6,472,371; 6,555,524; US 2002/0052328; US 2002/0061856; US 2002/0061857; US 2002/0077302; US 2002/0151507; US 2002/0156027; US 2003/0100518; US 2003/0100742; US 2003/0199458; US 2004/0077557; WO 99/11651; WO 99/21866; WO 99/21869; WO 99/35157; EP 1 114 826; and J. Med. Chem., 46, 2706 (2003). Additional relevant publications are cited hereinbelow. These and all documents cited herein are fully incorporated by reference herein for all purposes, including for the teachings, modifications, and method(s) of modifying the subject positions on macrolide rings in various combinations. Thus, derivatives can include, e.g., modifications at the C-2, C-3, C-6, C-9, C-10, C-11, C-12, and C-13 erythromycin positions, etc., and corresponding azalide derivatives.

### Cationic Antimicrobial Peptides

In some cases, the antimicrobial agent is a cationic antimicrobial peptide.

Cationic peptides having antimicrobial activity have been isolated from a wide variety of organisms. In nature, such peptides provide a defense mechanism against o microorganisms such as bacteria and yeast. Generally, these cationicpeptides are thought to exert their antimicrobial activity on bacteria by interacting with the cytoplasmic membrane to form channels or lesions. In gram-negative bacteria, they interact with surface lipopolysaccharide (LPS) to permeabilize the outer membrane, leading to self promoted uptake across the outer membrane and access to the cytoplasmic membrane. Examples of antimicrobial peptides include indolicidin, defensins, cecropins, and magainins. Methods of producing cationic antimicrobial peptides and variants thereof can be found in, for example, U.S. Patent Publication No.: U.S. Pat. Nos. 7,390,873, and 7,550,430, which are hereby incorporated by reference in their entirety.

### Hydrophilic Polymers

A conjugate of the present disclosure comprises an antimicrobial agent; and a hydrophilic polymer, wherein the antimicrobial agent is covalently linked, directly or via a linker, to the hydrophilic polymer. In some cases the the hydrophilic polymer is poly(ethylene glycol) (PEG), poly(ethylene oxide) (PEO), poly(N-isopropylacrylamide) (PNIPAM), poly(2-oxazoline), polyethylenimine (PEI), poly(vinyl alcohol) (PVA), or poly(vinylpyrrolidone) (PVP).

In some cases, the term “hydrophilic polymer” means a material that has the property of dissolving in, absorbing, or mixing easily with water, and comprises repeating units constituting an MW of at least 200 (e.g., PEG 200) up to 8,000 or more. In some cases, hydrophilic polymers include PEG as well as other materials, which can be used to solubilize antimicrobial agents of the present disclosure.

In some cases, the hydrophilic polymer has a molecular weight of from about 0.5 Da to about 2000 kDa. In some cases, the hydrophilic polymer has a molecular weight of from about 0.5 Da to about 1 kDa. In some cases, the hydrophilic polymer has a molecular weight of from about 1 Da to about 1.5 kDa. In some cases, the hydrophilic polymer has a molecular weight of from about 1.5 Da to about 2 kDa. In some cases, the hydrophilic polymer has a molecular weight of from about 2 Da to about 2.5 kDa. In some cases, the hydrophilic polymer has a molecular weight of from about 2.5 Da to about 3 kDa. In some cases, the hydrophilic polymer has a molecular weight of from about 3.5 Da to about 4 kDa. In some cases, the hydrophilic polymer has a molecular weight of from about 4 Da to about 4.5 kDa. In some cases, the hydrophilic polymer has a molecular weight of from about 4.5 Da to about 5 kDa. In some cases, the hydrophilic polymer has a molecular weight of from about 5.5 Da to about 6 kDa. In some cases, the hydrophilic polymer has a molecular weight of from about 6.5 Da to about 7 kDa. In some cases, the hydrophilic polymer has a molecular weight of from about 7.5 Da to about 8 kDa. In some cases, the hydrophilic polymer has a molecular weight of from about 8.5 Da to about 9 kDa. In some cases, the hydrophilic polymer has a molecular weight of from about 9.5 Da to about 10 kDa. In some cases, the hydrophilic polymer has a molecular weight of from about 10 Da to about 100 kDa.

In some cases, the hydrophilic polymer has a molecular weight of from about 100 Da to about 200 kDa. In some cases, the hydrophilic polymer has a molecular weight of from about 200 Da to about 300 kDa. In some cases, the hydrophilic polymer has a molecular weight of from about 300 Da to about 400 kDa. In some cases, the hydrophilic polymer has a molecular weight of from about 400 Da to about 500 kDa. In some cases, the hydrophilic polymer has a molecular weight of from about 500 Da to about 600 kDa. In some cases, the hydrophilic polymer has a molecular weight of from about 600 Da to about 700 kDa. In some cases, the hydrophilic polymer has a molecular weight of from about 700 Da to about 800 kDa. In some cases, the hydrophilic polymer has a molecular weight of from about 800 Da to about 900 kDa. In some cases, the hydrophilic polymer has a molecular weight of from about 900 Da to about 1000 kDa. In some cases, the hydrophilic polymer has a molecular weight of from about 1000 Da to about 1100 kDa. In some cases, the hydrophilic polymer has a molecular weight of from about 1200 Da to about 1300 kDa. In some cases, the hydrophilic polymer has a molecular weight of from about 1300 Da to about 1400 kDa. In some cases, the hydrophilic polymer has a molecular weight of from about 1400 Da to about 1500 kDa. In some cases, the hydrophilic polymer has a molecular weight of from about 1500 Da to about 1600 kDa. In some cases, the hydrophilic polymer has a molecular weight of from about 1600 Da to about 1700 kDa. In some cases, the hydrophilic polymer has a molecular weight of from about 1700 Da to about 1800 kDa. In some cases, the hydrophilic polymer has a molecular weight of from about 1800 Da to about 1900 kDa. In some cases, the hydrophilic polymer has a molecular weight of from about 1900 Da to about 2000 kDa.

In some cases, the molar ratio of antimicrobial agent to hydrophilic polymer is from 1:1 to 100:1. In some cases, the molar ratio of antimicrobial agent to hydrophilic polymer is from 1:1 to 2:1. In some cases, the molar ratio of antimicrobial agent to hydrophilic polymer is from 2:1 to 3:1. In some cases, the molar ratio of antimicrobial agent to hydrophilic polymer is from 3:1 to 4:1. In some cases, the molar ratio of antimicrobial agent to hydrophilic polymer is from 4:1 to 5:1. the molar ratio of antimicrobial agent to hydrophilic polymer can vary from about 5:1 to about 100:1, e.g., from about 5:1 to about 7:1, from about 7:1 to about 10:1, from about 10:1 to about 12:1, from about 12:1 to about 15:1, from about 15:1 to about 20:1, from about 20:1 to about 25:1, from about 25:1 to about 30:1, from about 30:1 to about 35:1, from about 35:1 to about 40:1, from about 40:1 to about 45:1, from about 45:1 to about 50:1, from about 50:1 to about 60:1, from about 60:1 to about 70:1, from about 70:1 to about 80:1, from about 80:1 to about 90:1, or from about 90:1 to about 100:1.

In some cases, suitable hydrophilic polymer can be of neutral, anionic, cationic or zwitterionic charge character, and include, for example, PEO, also known as PEG, and PEG derivatives (e.g., bisamino-propyl PEG), poly(N-vinylpyrolidinone), polyacrylamide, poly(acrylic acid), polyethyleneimine, polycarboxybetaine, polysulfobetaine, and derivatives thereof. In some cases, hydrophilic polymers have an affinity for water, as measured by a low water contact angle (<30° C.), and/or swellability or solubility in water. The amount of hydrophilic polymer component may vary. In some cases, the hydrophilic polymer component may be linear or branched.

In some cases, the hydrophilic polymer may function to increase hydrophilicity and/or circulation time) of a conjugate of the present invention. In some cases, the conjugate comprising an antimicrobial agent covalently linked to the hydrophilic polymer exhibits reduced toxicity compared to the toxicity exhibited by the antimicrobial agent in unconjugated form. In some cases, the conjugate comprising an antimicrobial agent covalently linked to the hydrophilic polymer has reduced side effects induced by the conjugate to the side effects induced by the antimicrobial agent in unconjugated form.

### Linkers

### PEG Linkers

In some cases, the hydrophilic polymer is PEG. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). Methods of conjugating PEG with molecules can be found in, for example, U.S. Patent Publication No.: 9238079, and 8535726, which is hereby incorporated by reference in its entirety.

The term “PEG” is used broadly to encompass any polyethylene glycol molecule, without regard to size or to modification at an end of the PEG, and can be represented by the formula:

X—O(CH₂CH₂O)ₙ₋₁CH₂CH₂OH, (1)

where n is 20 to 2300 and X is H or a terminal modification, e.g., a C₁₋₄ alkyl.

In some cases, a PEG used in the invention terminates on one end with hydroxy or methoxy, i.e., X is H or CH₃ (“methoxy PEG”). In some cases, the other end of the PEG, which is shown in formula (1) terminating in OH, covalently attaches to a linker moiety via an ether oxygen bond.

In some cases, when used in a chemical structure, the term “PEG” includes the formula (1) above without the hydrogen of the hydroxyl group shown, leaving the oxygen available to react with a free carbon atom of a linker of the invention to form an ether bond.

Any molecular mass for a PEG can be used as practically desired, e.g., from about 1,000 Daltons (Da) to 100,000 Da (n is 20 to 2300). The number of repeating units “n” in the PEG is approximated for the molecular mass described in Daltons. In some cases, the combined molecular mass of PEG on an activated linker is suitable for pharmaceutical use. Thus, the combined molecular mass of the PEG molecules should not exceed 100,000 Da. For example, if three PEG molecules are attached to a linker, where each PEG molecule has the same molecular mass of 12,000 Da (each n is about 270), then the total molecular mass of PEG on the linker is about 36,000 Da (total n is about 820). The molecular masses of the PEG attached to the linker can also be different, e.g., of three molecules on a linker two PEG molecules can be 5,000 Da each (each n is about 110) and one PEG molecule can be 12,000 Da (n is about 270).

In some cases, PEG is conjugated to the microbial agent. To conjugate PEG to the microbial agent, an activated linker covalently attached to one or more PEG molecules is reacted with an amino or imino group of an amino acid residue, in some cases, with an alpha amino group at the N-terminus of the antimicrobial agent, to form a mono-PEG-antimicrobial agent of the present disclosure.

A linker is “activated” if it is chemically reactive and ready for covalent attachment to an amino group on an amino acid residue. Any activated linker can be used in this invention provided it can accommodate one or more PEG molecules and form a covalent bond with an amino group of an amino acid residue under suitable reaction conditions. In some cases, the activated linker attaches to an alpha amino group in a highly selective manner over other attachment sites, e.g., epsilon amino group of lysine or imino group of histidine.

Activated PEG can be represented by the formula:

(PEG)_{b}-L′, (2)

where PEG (defined supra) covalently attaches to a carbon atom of the linker to form an ether bond, b is 1 to 9 (i.e. 1 to 9 PEG molecules can be attached to the linker), and L′ contains a reactive group (an activated moiety) which can react with an amino or imino group on an amino acid residue to provide a covalent attachment of the PEG to the antimicrobial agent.

In some cases, the activated linker (L′) of the invention contains an aldehyde of the formula RCHO, where R is a linear (straight chain) or branched C₁₋₁₁ alkyl. After covalent attachment of an activated linker to the antimicrobial agent, the linker (referred to as “-L-” in the structural formulas recited herein) between the antimicrobial agent and PEG contains 2 to 12 carbon atoms.

In some cases, the PEG activated linker is Propionaldehyde is an example of a preferred activated linker of this invention. PEG-propionaldehyde, represented in formula (3), is described in U.S. Pat. No. 5,252,714 and is commercially available from Shearwater Polymers (Huntsville, Ala.).

PEG-CH₂CH₂CHO (3)

If it is desirable to covalently attach more than one PEG molecule to an antimicrobial agent, then a suitable activated branched (also known as “multi-armed”) linker can be used. Any suitable branched PEG linker that covalently attaches two or more PEG molecules to an amino group on an amino acid residue of an antimicrobial agent, and in some cases, to an alpha amino group at the N-terminus, can be used. In some cases, a branched linker used in this invention contains two or three PEG molecules.

In some cases, a branched PEG linker used in the present disclosure can be a linear or branched aliphatic group that is hydrolytically stable and contains an activated moiety, e.g., an aldehyde group, which reacts with an amino group of an amino acid residue, as described above. In some cases, the aliphatic group of a branched linker contains 2 to 12 carbons. In some cases, an aliphatic group can be a t-butyl which contains as many as three PEG molecules on each of three carbon atoms (i.e., a total of 9 PEG molecules) and a reactive aldehyde moiety on the fourth carbon of the t-butyl.

Examples of activated, branched PEG linkers are also described in U.S. Pat. Nos. 5,643,575, 5,919,455, and 5,932,462. One having ordinary skill in the art can prepare modifications to branched PEG linkers as desired, e.g., addition of a reactive aldehyde moiety. Methods for the preparation of linkers for use in the present invention are well known in the art, e.g., see U.S. Pat. Nos. 5,643,575, 5,919,455, and 5,932,462. Activated PEG-linkers, such as PEG-aldehydes, can be obtained from a commercial source, e.g., Shearwater Polymers, (Huntsville, Ala.) or Enzon, Inc. (Piscataway, N.J.).

In some cases, the hydrophilic polymer is dextran. In some cases, the dextran may be branched. In some cases, the dextran straight chain consists of α1->6 glycosidic linkages between glucose molecules, while branches begin from α1->3 linkages (and in some cases, al->2 and α1->4 linkages as well). In some cases, Dextran 10, Dextran 40 and Dextran 70 (Mw=10,000, 40,000 and 70,000, respectively) may be applied at a concentration analogous to those described for PEG.

### Maltodextrin Polymers

The maltodextrins are obtained by acid and/or enzymatic hydrolysis of starch. Referring to the regulatory status, the maltodextrins have a dextrose equivalent (DE) of 1 to 20. Polymers based on maltodextrin can be found in, for example, International Patent Application Publication No.: WO2016004974, which is hereby incorporated by reference in its entirety.

In some cases, maltodextrin is generated by hydrolyzing a starch slurry with heat-stable α-amylase at about 85-90° C. until the desired degree of hydrolysis is reached, followed by inactivating the α-amylase by a second heat treatment. In some cases, the maltodextrin can be purified by filtration and then spray dried to a final product. Maltodextrins are typically characterized by their dextrose equivalent (DE) value, which is related to the degree of hydrolysis, and is defined as: DE=MW dextrose/number-averaged MW starch hydrosylatex 100. In some cases, maltodextrin is considered to have a molecular weight less than amylose.

A starch preparation that has been completely hydrolyzed to dextrose (glucose) has a DE of 100, whereas starch has a DE of about zero. A DE of greater than 0 but less than 100 characterizes the mean-average molecular weight of a starch hhydrosylate, and, in some cases, maltodextrins are considered to have a DE of less than 20. Maltodextrins of various molecular weights, including those in the range of about 500 Da to 5000 Da, are commercially available (for example, from CarboMer, San Diego Calif.).

In some cases the hydrophilic polymer of the conjugate of the present disclosure is a maltodextrin polymer. In some cases, the maltodextrin polymer is maltotriose, maltotetraose, maltopentaose, maltohexaose, maltoheptaose, maltooctaose, maltononaose, or maltodecaose.

In some cases, the maltodextrin polymer comprises from 2 to 20,000 α (1→4)-linked D-glucose subunits. In some cases, the maltodextrin polymer comprises from 2 to 400 α (1-4)-linked D-glucose subunits. In some cases, the maltodextrin polymer comprises from 400 to 800 α (1→4)-linked D-glucose subunits. In some cases, the maltodextrin polymer comprises from 800 to 1200 α (1→4)-linked D-glucose subunits. In some cases, the maltodextrin polymer comprises from 1200 to 1600 α (1→4)-linked D-glucose subunits. In some cases, the maltodextrin polymer comprises from 1600 to 2000 α (1→4)-linked D-glucose subunits. In some cases, the maltodextrin polymer comprises from 2000 to 2400 α (1→4)-linked D-glucose subunits. In some cases, the maltodextrin polymer comprises from 2400 to 2800α (1→4)-linked D-glucose subunits. In some cases, the maltodextrin polymer comprises from 2800 to 3000 α (1→4)-linked D-glucose subunits. In some cases, the maltodextrin polymer comprises from 3000 to 4000 α (1→4)-linked D-glucose subunits. In some cases, the maltodextrin polymer comprises from 4000 to 5000 α (1→4)-linked D-glucose subunits. In some cases, the maltodextrin polymer comprises from 5000 to 6000 α (1→4)-linked D-glucose subunits. In some cases, the maltodextrin polymer comprises from 6000 to 7000 α (1→4)-linked D-glucose subunits. In some cases, the maltodextrin polymer comprises from 7000 to 8000 α (1→4)-linked D-glucose subunits. In some cases, the maltodextrin polymer comprises from 8000 to 9000 α (1→4)-linked D-glucose subunits. In some cases, the maltodextrin polymer comprises from 9000 to 10000 α (1→4)-linked D-glucose subunits. In some cases, the maltodextrin polymer comprises from 10000 to 11000 α (1→4)-linked D-glucose subunits. In some cases, the maltodextrin polymer comprises from 11000 to 12000 α (1→4)-linked D-glucose subunits. In some cases, the maltodextrin polymer comprises from 12000 to 13000 α (1→4)-linked D-glucose subunits. In some cases, the maltodextrin polymer comprises from 13000 to 14000 α (1→4)-linked D-glucose subunits. In some cases, the maltodextrin polymer comprises from 14000 to 15000 α (1→4)-linked D-glucose subunits. In some cases, the maltodextrin polymer comprises from 15000 to 16000 α (1→4)-linked D-glucose subunits. In some cases, the maltodextrin polymer comprises from 16000 to 17000 α (1→4)-linked D-glucose subunits. In some cases, the maltodextrin polymer comprises from 17000 to 18000 α (1→4)-linked D-glucose subunits. In some cases, the maltodextrin polymer comprises from 18000 to 19000 α (1→4)-linked D-glucose subunits. In some cases, the maltodextrin polymer comprises from 19000 to 20000 α (1→4)-linked D-glucose subunits.

### Self-Emolative Linkers

In some cases, the antimicrobial agent is conjugated to the hydrophilic polymer via a cleavable linker. In some cases, the cleavable linker is a self-immolative linker. Compounds with with self-immolative linkers can be found in, for example, International Patent Application Publication No.: WO2014100762, and U.S. Patent Publication No.: 8399403, which are hereby incorporated by reference in their entirety.

In some cases, the self-immolative linker is a bifunctional chemical moiety which is capable of covalently linking together two spaced chemical moieties into a normally stable tripartate molecule, releasing one of said spaced chemical moieties from the tripartate molecule by means of enzymatic cleavage; and following said enzymatic cleavage, spontaneously cleaving from the remainder of the molecule to release the other of said spaced chemical moieties. In some cases, the self-immolative spacer is covalently linked at one of its ends to the peptide moiety and covalently linked at its other end to the chemical reactive site of the antimicrobial agent moiety whose derivatization inhibits pharmacological activity, so as to space and covalently link together the peptide moiety and the antimicrobial agent moiety into a tripartate molecule which is stable and pharmacologically inactive in the absence of the target enzyme, but which is enzymatically cleavable by such target enzyme at the bond covalently linking the spacer moiety and the peptide moiety to thereby effect release of the peptide moiety from the tripartate molecule. In such cases, such enzymatic cleavage will activate the self-immolating character of the spacer moiety and initiate spontaneous cleavage of the bond covalently linking the spacer moiety to the antimicrobial agent, to thereby effect release of the conjugate in pharmacologically active form.

In some cases, the self-immolative linker may be any self-immolative group. In some cases, the self-immolative linker has a substituted alkyl, unsubstituted alkyl, substituted heteroalkyl, unsubstituted heteroalkyl, unsubstituted heterocycloalkyl, substituted heterocycloalkyl, substituted and unsubstituted aryl, and substituted and unsubstituted heteroaryl.

In some cases, the antimicrobial agent conjugated to the hydrophilic polymer via a cleavable linker employs a hydrophilic self-immolative spacer moiety. In some cases, the self-immolative spacer moiety spaces and covalently links together the antimicrobial agent and the polymer and incorporates a hydrophilic group, which provides better solubility of the conjugate. In some cases, increased associated hydrophobicity of some enzyme-labile linkers can lead to aggregation of drug conjugates, particularly with strongly hydrophobic conjugates. In some cases, incorporation of a hydrophilic group into the linker, may lead to a decreased aggregation of the drug conjugate.

In some cases, the self-immolative linker is cleavable by a thiol. In some cases, the thiol thiol is glutathione. Examples of self-immolative linker molecules have been described in the literature are commercially available. See e.g. Amsberry, K. L., and Borchardt, R. T., The Lactonization Of 2′-Hydroxydydrocinnamic Acid Amides: A Potential Prodrug For Amines, J. Org. Chem 55(23):5867-5877 (1990); Dubowchik, G. M., et al., Efficient Mitocycin C Coupling with Stable p-Nitropheny-Benzy Carbonates Using N-Hydroxybenzotriazole as a Catalytic Additive, Tetrahedron Letters, 30(30):5261-5264 (1997); Rodrigues, M. L., et al., Synthesis And Beta-Lactamase-Mediated Activation Of A Cephalosporin-Taxol Prodrug, Chem Biol. 2(4):223-7 (1995); Shabat D., et al., Multiple Event Activation Of A Generic Prodrug Trigger By Antibody Catlaysis, Proc Natl Acad Sci USA 96(12): 6925-30 (1999); Shabat D., et al., In Vivo Activity In A Catalytic Antibody-Prodrug System: Antibody Catalyzed Etoposide Prodrug Activation For Selective Chemotherapy, Proc Natl Acad Sci USA 98(13):7528-33 (2001).

### Proteolytically Cleavable Linker

In some cases, the microbial agent is conjugated to the hydrophilic polymer via a cleavable linker. In some cases, the cleavable linker is a proteolytically cleavable linker. In some cases, the proteolytically cleavable linker is a water-hydrolyzable linker.

The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

For example, the proteolytically cleavable linker can comprise a matrix metalloproteinase (MMP) cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue), e.g., Pro-X-X-Hy-(Ser/Thr), e.g., Pro-Leu/Gln-Gly-Met-Thr-Ser (SEQ ID NO://) or Pro-Leu/Gln-Gly-Met-Thr (SEQ ID NO://). Another example of a protease cleavage site is a plasminogen activator cleavage site, e.g., a uPA or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYFQS (SEQ ID NO://), where the protease cleaves between the glutamine and the serine; or ENLYFQY (SEQ ID NO://), where the protease cleaves between the glutamine and the tyrosine; or ENLYFQL (SEQ ID NO://), where the protease cleaves between the glutamine and the leucine. Another example of a protease cleavage site that can be included in a proteolytically cleavable linker is an enterokinase cleavage site, e.g., DDDDK (SEQ ID NO://), where cleavage occurs after the lysine residue. Another example of a protease cleavage site that can be included in a proteolytically cleavable linker is a thrombin cleavage site, e.g., LVPR (SEQ ID NO://) (e.g., where the proteolytically cleavable linker comprises the sequence LVPRGS (SEQ ID NO://)). Additional suitable linkers comprising protease cleavage sites include linkers comprising one or more of the following amino acid sequences: LEVLFQGP (SEQ ID NO://), cleaved by PreScission protease (a fusion protein comprising human rhinovirus 3C protease and glutathione-S-transferase; Walker et al. (1994) Biotechnol. 12:601); a thrombin cleavage site, e.g., CGLVPAGSGP (SEQ ID NO://); SLLKSRMVPNFN (SEQ ID NO://) or SLLIARRMPNFN (SEQ ID NO://), cleaved by cathepsin B; SKLVQASASGVN (SEQ ID NO://) or SSYLKASDAPDN (SEQ ID NO://), cleaved by an Epstein-Barr virus protease; RPKPQQFFGLMN (SEQ ID NO://) cleaved by MMP-3 (stromelysin); SLRPLALWRSFN (SEQ ID NO://) cleaved by MMP-7 (matrilysin); SPQGIAGQRNFN (SEQ ID NO://) cleaved by MMP-9; DVDERDVRGFASFL SEQ ID NO://) cleaved by a thermolysin-like MMP; SLPLGLWAPNFN (SEQ ID NO://) cleaved by matrix metalloproteinase 2(MMP-2); SLLIFRSWANFN (SEQ ID NO://) cleaved by cathespin L; SGVVIATVIVIT (SEQ ID NO://) cleaved by cathepsin D; SLGPQGIWGQFN (SEQ ID NO://) cleaved by matrix metalloproteinase 1(MMP-1); KKSPGRVVGGSV (SEQ ID NO://) cleaved by urokinase-type plasminogen activator; PQGLLGAPGILG (SEQ ID NO://) cleaved by membrane type 1 matrixmetalloproteinase (MT-MMP);HGPEGLRVGFYESDVMGRGHARLVHVEEPHT (SEQ ID NO://) cleaved by stromelysin 3 (or MMP-11), thermolysin, fibroblast collagenase and stromelysin-1; GPQGLAGQRGIV (SEQ ID NO://) cleaved by matrix metalloproteinase 13 (collagenase-3); GGSGQRGRKALE (SEQ ID NO://) cleaved by tissue-type plasminogen activator(tPA); SLSALLSSDIFN (SEQ ID NO://) cleaved by human prostate-specific antigen; SLPRFKIIGGFN (SEQ ID NO://) cleaved by kallikrein (hK3); SLLGIAVPGNFN (SEQ ID NO://) cleaved by neutrophil elastase; and FFKNIVTPRTPP (SEQ ID NO://) cleaved by calpain (calcium activated neutral protease).

Suitable proteolytically cleavable linkers also include ENLYFQS (SEQ ID NO://), ENLYFQY (SEQ ID NO://), ENLYFQL (SEQ ID NO://), ENLYFQW (SEQ ID NO://), ENLYFQM (SEQ ID NO://), ENLYFQH (SEQ ID NO://), ENLYFQN (SEQ ID NO://), ENLYFQA (SEQ ID NO://), and ENLYFQQ (SEQ ID NO://).

Suitable proteolytically cleavable linkers also include NS3 protease cleavage sites such as: DEVVECS (SEQ ID NO://), DEAEDVVECS (SEQ ID NO://), EDAAEEVVECS (SEQ ID NO://).

Suitable proteolytically cleavable linkers also include calpain cleavage site, where suitable calpain cleavage sites include, e.g., PLFAAR (SEQ ID NO://) and QQEVYGMMPRD (SEQ ID NO://).

In some cases, the proteolytically cleavable linker comprises an amino acid sequence that is cleaved by a protease present in a bodily fluid of a mammalian individual. In some cases, the proteolytically cleavable linker comprises an amino acid sequence that is cleaved by a protease present in serum of an individual. In some cases, the proteolytically cleavable linker comprises an amino acid sequence that is cleaved by a protease present in extracellular fluid in an individual.

In some cases, the proteolytically cleavable linker comprises an amino acid sequence that is substantially not cleaved by any endogenous protease in a given cell (e.g., a eukaryotic cell; e.g., a mammalian cell; e.g., a particular type of mammalian cell). In some cases, the proteolytically cleavable linker comprises an amino acid sequence that is cleaved by a viral protease, and that is substantially not cleaved by any endogenous protease in a given cell (e.g., a eukaryotic cell; e.g., a mammalian cell; e.g., a particular type of mammalian cell). In some cases, the proteolytically cleavable linker comprises an amino acid sequence that is cleaved by a non-naturally occurring (e.g., engineered) protease, and that is substantially not cleaved by any endogenous protease in a given cell (e.g., a eukaryotic cell; e.g., a mammalian cell; e.g., a particular type of mammalian cell).

In some cases, the proteolytically cleavable linker comprises an amino acid sequence that is cleaved by a protease that is endogenous to a given cell (e.g., a bacterial cell).

### Polymyxin-Maltodextrin Polymer Conjugates

The present disclosure provides a conjugate comprising a polymyxin covalently linked to a maltodextrin polymer. In some cases, the polymyxin is a Colistin. In some cases, the polymyxin is colistin sulfate. In some cases, the polymyxin is colistin methane-sulfonate.

In some cases, colistin will be conjugated to maltodextrins. In some cases, the conjugate is a Colistin-Maltodextrin Conjugate (CMC). In some cases, the colistin is conjugated via a self-immolative linker. In some cases, the self-immolative linker is cleaved by glutathione (GSH). In some cases, the self-immolative linker is cleaved by other thiols in serum or in bacteria.

In some cases, CMC is initially cleaved by amylases in the serum. In some cases, the CMC generates maltodextrins 2-12 units in length, conjugated to colistin, which then target bacteria, via the maltodextrin transporter. After binding the cell surface of the bacteria, in some cases, thiols in the serum cleave the immolative linker and release unmodified colistin, which then causes bacterial cell death. In some cases, the CMC improve the treatment of drug resistant bacterial infections by targeting colistin to gram negative bacteria with maltodextrins.

In some cases, a maltodextrin of 28,000 molecular weight is coupled to azido acetic acid and then clicked onto the heterobifunctional cross-linker (4) that contains a terminal alkyne and a para-nitrophenyl activated hydroxyl. In some cases, the compound (4) contains a self-immolative disulfide linkage. In some cases, the self-immolative disulfide linkage of compound (4) can be cleaved in the presence of thiols such as glutathione (GSH). In some cases, the para-nitrophenyl activated maltodextrin is then conjugated to colistin and purified via dialysis.

### Compositions

The present disclosure provides compositions, including pharmaceutical compositions, comprising an anti-microbial agent/hydrophilic polymer conjugate of the present disclosure. The present disclosure provides compositions, including pharmaceutical compositions, comprising a polymyxin-maltodextrin conjugate of the present disclosure.

In some cases, a pharmaceutical composition composition of the present disclosure comprises: a) an anti-microbial agent/hydrophilic polymer conjugate of the present disclosure; and b) and a pharmaceutically acceptable excipient. In some cases, a pharmaceutical composition composition of the present disclosure comprises: a) a polymyxin-maltodextrin conjugate; and b) a pharmaceutically acceptable excipient. In some cases, the pharmaceutical composition is a liquid composition. In some cases, the pharmaceutical composition is an aerosol. In some cases, the pharmaceutical composition a gel, a semi-solid, or a solid. In some cases, the pharmaceutical composition is an aerosol. In some cases, the pharmaceutical composition a gel. In some cases, the pharmaceutical composition is an aerosol. In some cases, the pharmaceutical composition a semi-solid. In some cases, the pharmaceutical composition is an aerosol. In some cases, the pharmaceutical composition a solid.

In some cases, the conjugate present in the pharmaceutical composition in a concentration of from 0.01 μg/ml to 200 mg/ml. In some cases, the conjugate present in the pharmaceutical composition in a concentration of from 0.01 μg/ml to 0.05 mg/ml. In some cases, the conjugate present in the pharmaceutical composition in a concentration of from 0.05 μg/ml to 0.1 mg/ml. In some cases, the conjugate present in the pharmaceutical composition in a concentration of from 1 μg/ml to 2 mg/ml. In some cases, the conjugate present in the pharmaceutical composition in a concentration of from 2 μg/ml to 4 mg/ml. In some cases, the conjugate present in the pharmaceutical composition in a concentration of from 4 μg/ml to 6 mg/ml. In some cases, the conjugate present in the pharmaceutical composition in a concentration of from 6 μg/ml to 8 mg/ml. In some cases, the conjugate present in the pharmaceutical composition in a concentration of from 8 μg/ml to 10 mg/ml. In some cases, the conjugate present in the pharmaceutical composition in a concentration of from 10 μg/ml to 12 mg/ml. In some cases, the conjugate present in the pharmaceutical composition in a concentration of from 12 μg/ml to 14 mg/ml. In some cases, the conjugate present in the pharmaceutical composition in a concentration of from 14 μg/ml to 16 mg/ml. In some cases, the conjugate present in the pharmaceutical composition in a concentration of from 16 μg/ml to 18 mg/ml. In some cases, the conjugate present in the pharmaceutical composition in a concentration of from 18 μg/ml to 20 mg/ml. In some cases, the conjugate present in the pharmaceutical composition in a concentration of from 20 μg/ml to 25 mg/ml. In some cases, the conjugate present in the pharmaceutical composition in a concentration of from 25 μg/ml to 30 mg/ml. In some cases, the conjugate present in the pharmaceutical composition in a concentration of from 30 μg/ml to 35 mg/ml. In some cases, the conjugate present in the pharmaceutical composition in a concentration of from 35 μg/ml to 40 mg/ml. In some cases, the conjugate present in the pharmaceutical composition in a concentration of from 40 μg/ml to 45 mg/ml. In some cases, the conjugate present in the pharmaceutical composition in a concentration of from 45 μg/ml to 50 mg/ml. In some cases, the conjugate present in the pharmaceutical composition in a concentration of from 50 μg/ml to 55 mg/ml. In some cases, the conjugate present in the pharmaceutical composition in a concentration of from 55 μg/ml to 60 mg/ml. In some cases, the conjugate present in the pharmaceutical composition in a concentration of from 60 μg/ml to 65 mg/ml. In some cases, the conjugate present in the pharmaceutical composition in a concentration of from 65 μg/ml to 70 mg/ml. In some cases, the conjugate present in the pharmaceutical composition in a concentration of from 70 μg/ml to 75 mg/ml. In some cases, the conjugate present in the pharmaceutical composition in a concentration of from 75 μg/ml to 80 mg/ml. In some cases, the conjugate present in the pharmaceutical composition in a concentration of from 80 μg/ml to 85 mg/ml. In some cases, the conjugate present in the pharmaceutical composition in a concentration of from 85 μg/ml to 90 mg/ml. In some cases, the conjugate present in the pharmaceutical composition in a concentration of from 95 μg/ml to 100 mg/ml. In some cases, the conjugate present in the pharmaceutical composition in a concentration of from 100 μg/ml to 105 mg/ml. In some cases, the conjugate present in the pharmaceutical composition in a concentration of from 105 μg/ml to 110 mg/ml. In some cases, the conjugate present in the pharmaceutical composition in a concentration of from 110 μg/ml to 115 mg/ml. In some cases, the conjugate present in the pharmaceutical composition in a concentration of from 115 μg/ml to 120 mg/ml. In some cases, the conjugate present in the pharmaceutical composition in a concentration of from 120 μg/ml to 125 mg/ml. In some cases, the conjugate present in the pharmaceutical composition in a concentration of from 125 μg/ml to 130 mg/ml. In some cases, the conjugate present in the pharmaceutical composition in a concentration of from 130 μg/ml to 135 mg/ml. In some cases, the conjugate present in the pharmaceutical composition in a concentration of from 135 μg/ml to 140 mg/ml. In some cases, the conjugate present in the pharmaceutical composition in a concentration of from 140 μg/ml to 145 mg/ml. In some cases, the conjugate present in the pharmaceutical composition in a concentration of from 145 μg/ml to 150 mg/ml. In some cases, the conjugate present in the pharmaceutical composition in a concentration of from 150 μg/ml to 155 mg/ml. In some cases, the conjugate present in the pharmaceutical composition in a concentration of from 155 μg/ml to 160 mg/ml. In some cases, the conjugate present in the pharmaceutical composition in a concentration of from 160 μg/ml to 165 mg/ml. In some cases, the conjugate present in the pharmaceutical composition in a concentration of from 165 μg/ml to 170 mg/ml. In some cases, the conjugate present in the pharmaceutical composition in a concentration of from 170 μg/ml to 175 mg/ml. In some cases, the conjugate present in the pharmaceutical composition in a concentration of from 175 μg/ml to 180 mg/ml. In some cases, the conjugate present in the pharmaceutical composition in a concentration of from 180 μg/ml to 185 mg/ml. In some cases, the conjugate present in the pharmaceutical composition in a concentration of from 185 μg/ml to 190 mg/ml. In some cases, the conjugate present in the pharmaceutical composition in a concentration of from 190 μg/ml to 195 mg/ml. In some cases, the conjugate present in the pharmaceutical composition in a concentration of from 195 μg/ml to 200 mg/ml. In some cases, the conjugate present in the pharmaceutical composition in a concentration of from 200 μg/ml to 250 mg/ml. In some cases, the conjugate present in the pharmaceutical composition in a concentration of from 250 μg/ml to 500 mg/ml.

### Formulations

In some cases, the present disclosure is directed to pharmaceutical compositions comprising conjugates comprising an antimicrobial agent and a hydrophilic polymer according to the present disclosure, their salt forms, where the antimicrobial agent is covalently linked, directly or via a linker, to the hydrophilic polymer with one or more pharmaceutically acceptable carriers and excipients. An antimicrobial agent/hydrophilic polymer conjugate of the present disclosure is also referred to as an “active agent.” Carriers and excipients may facilitate processing of the active agent into preparations which can be used pharmaceutically and include e.g. diluting, filling, buffering, thickening, wetting, dispersing, solubilizing, suspending, emulsifying, binding, stabilizing, disintegrating, encapsulating, coating, embedding, lubricating, colouring, and flavouring agents as well as absorbents, absorption enhancers, humefactants, preservatives and the like, well-known to a person skilled in the art.

In some cases, pharmaceutical compositions include compositions wherein the active agent is contained in an amount effective to achieve the intended purpose. More specifically, a therapeutically effective amount means an amount of compound effective to treat, prevent, alleviate or ameliorate symptoms of pathology or prolong the survival of the subject being treated at a reasonable benefit to risk ratio applicable to any medical treatment. Determination of a therapeutically effective amount is well within the capability of those skilled in the art of medicine.

In some cases, a composition of the present disclosure may be produced by processes well known in the art, e.g. by means of conventional mixing, dissolving, encapsulating, entrapping, lyophilizing, emulsifying and granulating processes. The proper formulation is dependent upon the route of administration chosen, and the pharmaceutical composition can be formulated for immediate release or slow release (e.g. in order to prolong the therapeutic effect and/or improve tolerability). Furthermore, the formulations may conveniently be presented in unit dosage form by methods known in the art of pharmacy.

In some cases, pharmaceutical compositions according to the present disclosure include, but are not limited to, those intended for intravenous, intramuscular, oral, or topical administration as well as those being administered as a suppository or as an inhalable aerosol. The compositions include intravenous, intramuscular, intraperitoneal, subcutaneous, intramedullary, intrathecal, intraventricular, intranasal, or intraocular injections, inhalable aerosols as well as those intended for rectal, oral, intravaginal, transmucosal or transdermal delivery.

In some cases, for parenteral administration (e.g. by bolus injection, fast running infusions, or slow infusions), an active agent of the present disclosure may be formulated as a suitable salt or ester forms in sterile aqueous solutions, in some cases, physiologically compatible fluids such as saline, 5% dextrose, Ringer's solution, and Hank's solution. In some cases, the formulation may also include organic solvents such as propylene glycol, polyethylene glycol, propylene glycol or related compounds as well as preservatives and surfactants.

In some cases, pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

In some cases, the pharmaceutical compositions for parental administration may be suspensions or emulsions in oily or aqueous vehicles, and may contain formulatory agents such as suspending, stabilizing and/or dispersing agents. Suitable lipophilic vehicles and solvents include fatty oils such as natural and/or synthetic fatty acids esters, such as ethyl oleate and triglycerides, or liposomes. The suspensions may contain substances, which increase the viscosity of the suspension, such as sodium carboxymethyl cellulose, sorbitol or dextran.

In some cases, the parenteral compositions can be presented in unit-dose or multi-dose sealed containers, such as ampules and vials, and can be stored in a freeze-dried (lyophilized) condition requiring only the addition of the sterile liquid excipient, for example, water, for injections, immediately prior to use.

In some cases, the conjugate is administered orally. For oral administration, solid form preparations include, but are not limited to, e.g. powders, tablets, pills, dragees, lozenges, capsules, cachets, and microgranular preparations. Pharmaceutical preparations can be made using a solid excipient, optionally grinding the resulting mixture, and processing the mixture of granules, after adding suitable auxiliaries if desired, to obtain tablets or dragee cores. A solid carrier/excipient can be one or more substances which may also act as diluents, solubilizers, lubricants, suspending agents, binders, preservatives, flavouring agents, wetting agents, tablet disintegrating agents, or an encapsulating material. Suitable carriers include, but are not limited to, magnesium carbonate, magnesium stearate, talc, dextrose, lactose, pectin, starch, gelatin, tragacanth, methylcellulose, sodium carboxymethylcellulose, a low melting wax, cocoa butter, and the like.

In some cases, liquid preparations suitable for oral administration include, e.g., aqueous solutions, syrups, elixirs, aqueous suspensions, emulsions and gels. Aqueous solutions can be prepared by dissolving the active component in water and adding suitable stabilizing and thickening agents as well as colorants and flavours. Aqueous suspensions can be prepared by dispersing the finely divided active component in water with viscous material, such as natural or synthetic gums, resins, methylcellulose, sodium carboxymethylcellulose, and other well known suspending agents. Emulsions may be prepared in solutions in aqueous propylene glycol solutions or may contain emulsifying agents such as lecithin, sorbitan monooleate or acacia.

In some cases, an active agent of the present disclosure is formulated for topical administration. In some cases, the active agent is admixed under sterile conditions with pharmaceutically acceptable carriers/excipients, including any needed buffering agents and preservatives. In some cases, ointments, creams and lotions may, for example, be formulated with an aqueous or oily base with the addition of suitable emulsifying, dispersing, suspending, thickening, stabilizing, or coloring agents. In some cases, commonly used excipients include animal and vegetable fats and oils, waxes, paraffins, starch, cellulose derivatives, tragacanth, and polyethylene glycol.

In some cases, an active agent of the present disclosure is formulated in a topical formulation. Suitable topical formulations include, but are not limited to, ear-drops, eye-drops, and transdermal patches. For transdermal as well as transmucosal administration, penetrants generally known in the art may be used in the formulation.

In some cases, a conjugate of the present disclosure is administered by inhalation. In some cases, administration by inhalation include a conjugate of the present disclosure delivered in the form of an aerosol spray presentation from a ventilator, pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g. gelatin for use in an inhaler or insufflator may be formulated containing a powder mix of the compound and a suitable powder base such as lactose or starch.

In some cases, a conjugate of the present disclosure and the combinations described above may also be formulated in rectal compositions such as retention enemas or suppositories, using conventional suppository bases such as cocoa butter, other glycerides, polyethylene glycol, or a suppository wax.

The present disclosure also relates to a method for using conjugates of the present disclosure as a part of the clinical treatment of (or a preventive prophylactic regimen for) human or animal subjects suffering of an infectious disease, and comprises administering to said subject an therapeutically effective dose of at least one derivative according to the present disclosure.

### Methods of Inhibiting Bacterial Growth

The present disclosure provides methods of inhibiting growth of a bacterium. The methods generally involve contacting the bacterium with a polymyxin-maltodextrin conjugate of the present disclosure. The present disclosure provides methods of treating a bacterial infection in an individual. The methods generally involve administering to the individual an effective amount of an anti-microbial agent/hydrophilic polymer conjugate of the present disclosure. In some cases, the methods involve administering to the individual an effective amount of a polymyxin-maltodextrin conjugate of the present disclosure.

In some cases, the minimum inhibitory concentration of a conjugate of the present disclosure is from about 0.001 μg/ml to 10 μg/ml of unconjugated antimicrobial agent equivalents. In some cases, the minimum inhibitory concentration of a conjugate of the present disclosure is from about 0.01 μg/ml to 0.005 μg/ml of unconjugated antimicrobial agent equivalents. In some cases, the minimum inhibitory concentration of a conjugate of the present disclosure is from about 0.005 μg/ml to 0.01 μg/ml of unconjugated antimicrobial agent equivalents. In some cases, the minimum inhibitory concentration of a conjugate of the present disclosure is from about 0.01 μg/ml to 10 μg/ml of unconjugated antimicrobial agent equivalents. In some cases, the minimum inhibitory concentration of a conjugate of the present disclosure is from about 0.01 μg/ml to 0.05 μg/ml of unconjugated antimicrobial agent equivalents. In some cases, the minimum inhibitory concentration of a conjugate of the present disclosure is from about 0.05 μg/ml to 0.1 μg/ml of unconjugated antimicrobial agent equivalents. In some cases, the minimum inhibitory concentration of the conjugate is from about 0.1 μg/ml to 0.5 μg/ml of unconjugated antimicrobial agent equivalents. In some cases, the minimum inhibitory concentration of a conjugate of the present disclosure is from about 0.5 μg/ml to 1 μg/ml of unconjugated antimicrobial agent equivalents. In some cases, the minimum inhibitory concentration of a conjugate of the present disclosure is from about 1 μg/ml to 2 μg/ml of unconjugated antimicrobial agent equivalents. In some cases, the minimum inhibitory concentration of a conjugate of the present disclosure is from about 2 μg/ml to 3 μg/ml of unconjugated antimicrobial agent equivalents. In some cases, the minimum inhibitory concentration of a conjugate of the present disclosure is from about 3 μg/ml to 4 μg/ml of unconjugated antimicrobial agent equivalents. In some cases, the minimum inhibitory concentration of a conjugate of the present disclosure is from about 4 μg/ml to 5 μg/ml of unconjugated antimicrobial agent equivalents. In some cases, the minimum inhibitory concentration of a conjugate of the present disclosure is from about 5 μg/ml to 6 μg/ml of unconjugated antimicrobial agent equivalents. In some cases, the minimum inhibitory concentration of a conjugate of the present disclosure is from about 6 μg/ml to 7 μg/ml of unconjugated antimicrobial agent equivalents. In some cases, the minimum inhibitory concentration of a conjugate of the present disclosure is from about 7 μg/ml to 8 μg/ml of unconjugated antimicrobial agent equivalents. In some cases, the minimum inhibitory concentration of a conjugate of the present disclosure is from about 8 μg/ml to 9 μg/ml of unconjugated antimicrobial agent equivalents. In some cases, the minimum inhibitory concentration of a conjugate of the present disclosure is from about 9 μg/ml to 10 μg/ml of unconjugated antimicrobial agent equivalents. In some cases, the minimum inhibitory concentration of a conjugate of the present disclosure is from about 10 μg/ml to 15 μg/ml of unconjugated antimicrobial agent equivalents. In some cases, the minimum inhibitory concentration of a conjugate of the present disclosure is from about 15 μg/ml to 20 μg/ml of unconjugated antimicrobial agent equivalents.

In some cases, a conjugate of the present disclosure is a polymyxin-maltodextrin conjugate. In some cases, the minimum inhibitory concentration of the polymyxin-maltodextrin conjugate is from about 0.01 μg/ml to 10 μg/ml.

In some cases, the bacterium is a gram-negative bacterium. In some cases, the bacterium is a gram-positive bacterium. In some cases, the bacterium is resistant to a carbapenem antibiotic. In some cases, the bacterium is resistant to more than one antibiotic.

Carbapenem antibiotics are members of the beta lactam class of antibiotics. Carbapenem antibiotics are used for the treatment of infections known to be caused by multidrug-resistant bacteria. The term “carbapenem” is defined as the 4:5 fused ring lactam of penicillins with a double bond between C-2 and C-3 but with the substitution of carbon for sulfur at C-1. See e.g. Papp-Wallace et al. Antimicrob Agents Chemother. 2011 November; 55(11): 4943-4960.

### Dosages

The formulation of a conjugate of the present disclosure and its subsequent administration (dosing) is within the skill of those in the art. Dosing can be dependent on one or more of several criteria, including severity and responsiveness of the disease state or infection to be treated, with the course of treatment lasting from several days to several months, or until a cure is effected or a diminution of the disease state or infection is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual active agents, and can generally be estimated based on EC50s found to be effective in vitro and in vivo animal models.

For example, in some cases, a suitable dose of a conjugate of the present disclosure is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, from 100 μg to 1 mg per kg of body weight, from 0.5 mg to 200 mg per kg of body weight, from 0.5 mg to 190 mg per kg of body weight, from 0.5 to 180 mg per kg of body weight, from 0.5 mg to 170 mg per kg of body weight, from 0.5 mg to 160 mg per kg of body weight, from 0.5 mg to 150 mg per kg of body weight, 0.5 mg to 140 mg per kg of body weight, from 0.5 mg to 130 mg per kg of body weight, from 0.5 mg to 120 mg per kg of body weight, from 0.5 mg to 110 mg per kg of body weight, from 0.5 mg to 110 mg per kg of body weight, from 0.5 mg to 100 mg per kg of body weight, from 1 mg to 100 mg per kg of body weight, from 1 mg to 90 mg per kg of body weight, from 1 mg to 80 mg per kg of body weight, from 1 mg to 70 mg per kg of body weight, from 1 mg to 60 mg per kg of body weight, from 1 mg to 50 mg per kg of body weight, from 1 mg to 40 mg per kg of body weight, from 1 mg to 30 mg per kg of body weight, from 1 mg to 20 mg per kg of body weight, from 1 mg to 10 mg per kg of body weight, from 1 mg to 50 mg per kg of body weight, from 1 mg to 2.5 mg per kg of body weight, or from 1 mg to 1.5 mg per kg of body weight. Persons of ordinary skill in the art can easily estimate repetition rates for dosing based on measured residence times and concentrations of the drug in bodily fluids or tissues. In some cases, the conjugate is administered in a dose of from about 1 mg/kg per day to about 100 mg/kg per day, wherein the dose is based on the amount of equivalents of the unconjugated antimicrobial agent.

In some cases, multiple doses of a conjugate of the present disclosure are administered. The frequency of administration of an active agent can vary depending on any of a variety of factors, e.g., severity of the symptoms, etc. For example, in some embodiments, a conjugate of the present disclosure is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). In some cases, a conjugate of the present disclosure is administered once per day. In some cases, a conjugate of the present disclosure is administered continuously over time. For example, in some cases, a conjugate of the present disclosure is administered continuously (e.g., via intravenous administration) over a period of time of from about 12 hours to 7 days, from about 12 hours to about 24 hours, from about 1 day to about 2 days, from about 2 days to about 4 days, or from about 4 days to about 7 days.

The duration of administration of a conjugate of the present disclosure, e.g., the period of time over which an active agent is administered, can vary, depending on any of a variety of factors, e.g., patient response, etc. For example, an active agent can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Routes of Administration

A method of the present disclosure for treating a bacterial infection in an individual comprises administering to the individual an effective amount of a conjugate of the present disclosure. A conjugate is administered to an individual using any available method and route suitable for drug delivery, including in vivo and ex vivo methods, as well as systemic and localized routes of administration.

A conjugate of the present disclosure is administered to an individual using any available method and route suitable for drug delivery, including in vivo and ex vivo methods, as well as systemic and localized routes of administration. In some cases, the conjugate is administered via oral administration. In some cases, conjugate is administered via pulmonary administration. In some cases, the conjugate is administered via inhalational administration. In some cases, the conjugate is administered via intranasal administration. In some cases, the conjugate is administered via mucosal administration. In some cases, the conjugate is administered via topical administration. In some cases, the conjugate is administered via ocular administration. In some cases, the conjugate is administered via intravenous administration. In some cases, the conjugate is administered via subcutaneous administration.

Conventional and pharmaceutically acceptable routes of administration include intranasal, intramuscular, intratracheal, intracranial, subcutaneous, intradermal, topical application, intravenous, rectal, nasal, oral and other enteral and parenteral routes of administration. Routes of administration may be combined, if desired, or adjusted depending upon the conjugate and/or the desired effect. A conjugate of the present disclosure can be administered in a single dose or in multiple doses. In some cases, a conjugate of the present disclosure is administered orally. In other cases, a conjugate of the present disclosure is administered intravenously. In other cases, a conjugate of the present disclosure is administered via an inhalational route. In other cases, a conjugate of the present disclosure is administered intramuscularly. In other cases, a conjugate of the present disclosure is administered topically to the skin. In other cases, a conjugate of the present disclosure is administered intradermally. In other cases, the composition is administered subcutaneously. In other cases, a conjugate of the present disclosure is administered transdermally.

A conjugate of the present disclosure can be administered to a host using any available conventional methods and routes suitable for delivery of conventional drugs, including systemic or localized routes. In general, routes of administration contemplated by the present disclosure include, but are not necessarily limited to, enteral, parenteral, or inhalational routes. In some cases, a conjugate of the present disclosure is administered orally. In some cases, a conjugate of the present disclosure is administered topically. In some cases, a conjugate of the present disclosure is administered intradermally. In some cases, a conjugate of the present disclosure is administered subcutaneously.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the conjugate. Where systemic delivery is desired, administration typically involves invasive or systemically absorbed topical or mucosal administration of pharmaceutical preparations.

A conjugate of the present disclosure gent can also be delivered to the subject by enteral administration. Enteral routes of administration include, but are not necessarily limited to, oral and rectal (e.g., using a suppository) delivery. In some cases, the conjugate may be administered in an aerosolized or nebulized form.

The present disclosure also provides methods of inhibiting growth of a bacterium, wherein the methods include contacting a conjugate of the present disclosure with a bacterium.

### Bacteria

In some cases, a conjugate of the present disclosure inhibits growth of a bacterium. In some cases, a method of inhibiting browth of a bacterium includes contacting the bacterium with the conjugate. As discussed above, a conjugate of the present disclosure can be administered to an individual having a bacterial infection.

In some cases, the bacterium is Pseudomonas aeruginosa, Klebsiella pneumoniae, Acinetobacter baumannii, Escherichia coli, or Staphylococcus aureus.

In some cases, the bacterium is a gram-negative bacterium. Examples of gram-negative bacteria include, but are not limited to: Escherichia coli, Pseudoimonas aeruginosa, Klebsiella pneumoniae, Acinetobacter. Acinetobacter baumannii, and Neisseria gonorrhoeae.

In some cases, the bacterium is a gram-positive bacterium. Examples of gram-positive bacteria include, but are not limited to: Streptococcus pneumoniae, Staphylococcus aureus, Streptococcus pyogenes, Enterococcus faecalis, and Enterococcus faecium.

In some cases, the bacterium is resistant to a carbapenem antibiotic. In some cases, the bacterium is resistant to more than one antibiotic.

### Combination

In some cases, a conjugate of the present disclosure is administered in combination therapy with at least one additional therapeutic agent. In some cases, the one additional therapeutic agent is an antibiotic that is different from the antimicrobial agent in the conjugate.

In some cases, the at least one additional therapeutic agent is an antibiotic. In some cases, the at least one additional therapeutic agent is an antibiotic that has antimicrobial activity against Gram-negative bacteria, but little or no antimicrobial activity against Gram-positive bacteria. In some cases, the at least one additional therapeutic agent is an antibiotic. In some cases, the at least one additional therapeutic agent is an antibiotic that has antimicrobial activity against Gram-positive bacteria, but little or no antimicrobial activity against Gram-negative bacteria. In some cases, the at least one additional therapeutic agent is an antibiotic that has antimicrobial activity against both Gram-positive bacteria and Gram-negative bacteria.

In some cases, the at least one additional therapeutic agent is an antibiotic that has antimicrobial against Gram-positive bacteria (e.g., exhibits antimicrobial activity at an MIC that is less than 10 μg/ml, less than 5 μg/ml, or less than 1 μg/ml), and moderate activity (e.g., exhibits antimicrobial activity at an MIC that is less than 32 μg/ml, less than 64 μg/ml, or less than 128 μg/ml) against Gram-negative bacteria, where such antibiotics include rifampicin, novobiocin, macrolides, pleuromutilins.

In some cases, the at least one additional therapeutic agent is an antibiotic that has antimicrobial activity against Gram-negative bacteria, where such antibiotics include beta-lactams, tetracyclines, aminoglycosides, and quinolones.

In some cases, the at least one additional therapeutic agent is an antibiotic that is essentially inactive (e.g., exhibits antimicrobial activity, if any, at an MIC value that is at least 32 μg/ml, at least 64 μg/ml, at least 128 μg/ml, or at least 256 μg/ml) against Gram-negative bacteria. Examples of such antibiotics include fusidic acid, oxazolidinines (e.g. linezolid), glycopeptides (e.g. vancomycin), daptomycin and lantibiotics.

In some cases, the at least one additional therapeutic agent is an antibiotic that exhibits antimicrobial activity at an MIC value against a given bacterium that is less than 10 μg/ml, less than 5 μg/ml, or less than 1 μg/ml. In some cases, the at least one additional therapeutic agent is an antibiotic that exhibits antimicrobial activity at an MIC value against a given bacterium that is less than 32 μg/ml, less than 64 μg/ml, or less than 128 μg/ml. In some cases, the at least one additional therapeutic agent is an antibiotic that exhibits antimicrobial activity at an MIC value against a given bacterium that is at least 4 μg/ml, at least 8 μg/ml, at least 16 μg/ml, or at least 32 μg/ml. In some cases, the at least one additional therapeutic agent is an antibiotic that exhibits antimicrobial activity at an MIC value against a given bacterium that is at least 32 μg/ml, at least 64 μg/ml, at least 128 μg/ml, or at least 256 μg/ml.

In some cases, the at least one additional therapeutic agent is an antibiotic, where suitable antibiotics include but are not limited to any one or more of Aminocoumarins (such as Novobiocin, Albamycin, Coumermycin and Clorobiocin), Aminoglycosides (such as Amikacin, Apramycin, Gentamicin, Kanamycin, Neomycin, Netilmicin, Tobramycin, Paromomycin and Spectinomycin), Ansamycins (such as Geldanamycin, Herbimycin, Rifaximin and Streptomycin), Carbapenems (such as Ertapenem, Doripenem, Cilastatin (Imipenem) and Meropenem), Cephalosporins (such as Cefadroxil, Cefazolin, Cefalothin (Cefalotin), Cefalexin, Cefaclor, Cefamandole, Cefoxitin, Cefprozil, Cefuroxime, Cefixime, Cefdinir, Cefoperazone, Cefotaxime, Cefpodoxime, Ceftazidime, Ceftibuten, Ceftizoxime, Ceftriaxone, Cefepime, Ceftaroline fosamil and Ceftobiprole) Glycopeptides (such as Teicoplanin, Vancomycin and Telavancin), Lincosamides (such as Clindamycin and Lincomycin), Lipopeptides (such as Daptomycin), Macrolides (such as Azithromycin, Clarithromycin, Dirithromycin, Erythromycin, Roxithromycin, Troleandomycin, Telithromycin and Spiramycin), Monobactams (such as Aztreonam), Nitrofurans (such as Furazolidone and Nitrofurantoin), Oxazolidonones (such as Linezolid, Posizolid, Radezolid and Torezolid), Penicillins (such as Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin and Ticarcillin), Penicillin combinations (such as Amoxicillin/clavulanate, Ampicillin/sulbactam, Piperacillin/tazobactam and Ticarcillin/clavulanate), Polyethers (such as Monensin), Polypeptides (such as Bacitracin, Colistin and Polymyxin B), Quinolones (such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin); Sulfonamides (such as Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Sulfamethoxazole (Co-trimoxazole, TMP-SMX, ‘Trimethoprim’) and Sulfonamidochrysoidine), Tetracyclines (such as Demeclocycline, Doxycycline, Minocycline, Oxytetracycline and Tetracycline) and others (such as Clofazimine, Dapsone, Capreomycin, Cycloserine, Ethambutol, Ethionamide, Isoniazid, Pyrazinamide, Rifampicin (Rifampin), Rifabutin, Rifapentine, Streptomycin, Arsphenamine, Chloramphenicol, Fosfomycin, Fusidic acid, Metronidazole, Mupirocin, Platensimycin, Quinupristin (Dalfopristin), Thiamphenicol, Tigecycline, Tinidazole and Trimethoprim).

In some cases, the at least one additional therapeutic agent is an antibiotic selected from rifampicin, rifabutin, rifalazil, rifapentine, rifaximin, oxacillin, methicillin, ampicillin, cloxacillin, carbenicillin, piperacillin, tricarcillin, flucloxacillin, nafcillin, azithromycin, clarithromycin, erythromycin, telithromycin, cethromycin, solithromycin, aztreonam, BAL30072, meropenem, doripenem, imipenem, ertapenem, biapenem, tomopenem, panipenem, tigecycline, omadacycline, eravacycline, doxycycline, minocycline, ciprofloxacin, levofloxacin, moxifloxacin, delafloxacin, fusidic acid, novobiocin, teichoplanin, telavancin, dalbavancin, and oritavancin, and pharmaceutically acceptable salts and solvates thereof. In some cases, the at least one additional therapeutic agent is an antibiotic selected from the group consisting of rifampicin (rifampin), rifabutin, rifalazil, rifapentine, rifaximin, aztreonam, oxacillin, novobiocin, fusidic acid, azithromycin, ciprofloxacin, meropenem, tigecycline, erythromycin, clarithromycin and mupirocin, and pharmaceutically acceptable salts, solvates and prodrug forms thereof. In some cases, the at least one additional therapeutic agent is an antibiotic selected from the group consisting of rifampicin, fusidic acid, novobiocin, oxacillin, azithromycin, aztreonam, meropenem, tigecycline, ciprofloxacin, and vancomycin. In some cases, the at least one additional therapeutic agent is an antibiotic selected from the group consisting of rifampicin, fusidic acid, novobiocin, oxacillin, azithromycin, aztreonam, meropenem, tigecycline, and ciprofloxacin.

In some cases, the at least one additional therapeutic agent is an antibiotic selected from the following classes of agent: 1) Rifampicin family, including rifampicin, rifabutin, rifalazil, rifapentine, and rifaximin; 2) Oxacillin family, including oxacillin, methicillin, ampicillin, cloxacillin, carbenicillin, piperacillin, tricarcillin, flucloxacillin, and nafcillin; 3) Azithromycin family, including azithromycin, clarithromycin, erythromycin, telithromycin, cethromycin, and solithromycin; 4) Aztreonam family, including aztreonam and BAL30072; 5) Meropenem family, including meropenem, doripenem, imipenem, ertapenem, biapenem, tomopenem, and panipenem; 6) Tigecycline family, including tigecycline, omadacycline, eravacycline, doxycycline, and minocycline; 7) Ciprofloxacin family, including ciprofloxacin, levofloxacin, moxifloxacin, and delafloxacin; 8) Fusidic acid; 9) Novobiocin; 10) Vancomycin family, including vancomycin, teichoplanin, telavancin, dalbavancin, oritavancin, for example including teichoplanin, telavancin, dalbavancin, and oritavancin, and pharmaceutically acceptable salts and solvates of any of the foregoing.

In some cases, the at least one additional therapeutic agent is an antibiotic selected from the following classes of agent: 1) Chloramphenicol; 2) Clindamycin; 3) Oxazolidinone family including linezolid, torezolid, and radezolid; 4) Aminoglycoside family including amikacin, arbekacin, gentamicin, kanamycin, neomycin, netilmycin, paromomycin, streptomycin, tobramycin, apramycin, etimycin, and plazomycin; 5) Daptomycin; 6) Synercid; 7) Pleuromutilin family, including retapamulin, and BC-3781; 8) Lantibiotic family, including nisin, mersacidin, actagardine, deoxyactagardine B, NVB302, NVB333, Mu1140, and microbisporicin; 9) Cephalosporin family, including ceftaroline, ceftobiprole, ceftriaxone, ceftolozone, cefepime, cefuroxime, cefpodoxime, cefdinir, cefixime, cefotaxime, and ceftazidime; 10) Sulbactam; and 11) Sulopenem, and pharmaceutically acceptable salts and solvates of any of the foregoing.

In some cases, the at least one additional therapeutic agent is an antibiotic selected from meropenem, doripenem, imipenem, ertapenem, biapenem, tomopenem, and panipenem, and pharmaceutically acceptable salts and solvates thereof.

In some cases, the at least one additional therapeutic agent is an antibiotic selected from vancomycin, fosfomycin, rifamycin, a beta-lactam (such as a cephalosporin or carbapenem), an aminoglycoside, a macrolide, a tetracyline, a lipopeptide, and an oxazolidinone.

In some cases, the at least one additional therapeutic agent is an antifungal compound. Suitable antifungal compounds (antimycotics) include but are not limited to any one or more of Polyene antifungals (such as Amphotericin B, Candicidin, Filipin, Hamycin, Natamycin, Nystatin and Rimocidin), Imidazoles (such as Bifonazole, Butoconazole, Clotrimazole, Econazole, Fenticonazole, Isoconazole, Ketoconazole, Miconazole, Omoconazole, Oxiconazole, Sertaconazole, Sulconazole and Tioconazole), Triazoles (such as Albaconazole, Fluconazole, Isavuconazole, Itraconazole, Posaconazole, Ravuconazole, Terconazole and Voriconazole), Thiazoles (such as Abafungin), Allylamines (such as Amorolfin, Butenafine, Naftifine and Terbinafine), Echinocandins (such as Anidulafungin, Caspofungin and Micafungin) and others such as Benzoic acid, Ciclopirox, Flucytosine or 5-fluorocytosine, Griseofulvin, Haloprogin, Polygodial, Tolnaftate, Undecylenic acid, and Crystal violet.

### Subjects Suitable for Treatment

The present disclosure relates to a method for using conjugates comprising an antimicrobial agent and a hydrophilic polymer as a part of the clinical treatment of (or a preventive prophylactic regimen for) human or non-human animal subjects suffering of an infectious disease (i.e., a Gram-negative bacterial infection), and comprises administering to said subject an therapeutically effective dose of at least one conjugate according to the present disclosure.

Conjugates according to the present disclosure may inhibit the growth of antibacterial agents clinically important Gram-negative bacteria such as those belonging to the genus of Acinetobacter, Aeromonas, Alcaligenes, Bordetella, Branhamella, Campylobacter, Citrobacter, Enterobacter, Escherichia, Francisella, Fusobacterium, Haemophilus, Helicobacter, Klebsiella, Legionella, Moraxella, Pasteurella, Plesiomonas, Pseudomonas, Salmonella, Serratia, Shigella, and Yersinia species. The bacteria may be, for example, Escherichia coli, Klebsiella pneumoniae, Klebsiella oxytoca, Enterobacter cloacae, Enterobacter aerogenes, other species of Enterobacter, Citrobacter freundii, Pseudomonas aeruginosa, other species of Pseudomonas, Acinetobacter baumannii, as well as many other species of non-fermentative Gram-negative bacteria. The bacteria also include Helicobacter pylori, as well as other clinically important Gram-negative bacteria.

In some cases, the bacterial infections to be treated include, but are not limited to, for example, bacteremia, septicemia, skin and soft tissue infection, pneumonia, meningitis, infections in the pelveoperitoneal region, forging body infection, fever in hematological patient, infection associated with an intravenous line or other catheter, canyl and/or device, infection in gastrointestinal tract, in the eye, or in the ear, superficial skin infection, and colonization of gastrointestinal tract, mucous membranes and/or skin by potentially noxious bacteria.

In some cases, the bacterial infectious diseases include, but are not limited to, severe hospital-acquired infections, infections of the immunocompromised patients, infections of the organ transplant patients, infections at the intensive care units (ICU), severe infections of burn wounds, severe community-acquired infections, infections of cystic fibrosis patients, as well as infections caused by multi-resistant Gram-negative bacteria. Subjects suitable for treatment can be found in US Patent Application Publication No.: 2014/0162937, which is hereby incorporated by reference in its entirety.

Those skilled in the art of medicine can readily optimize effective dosages and administration regimens for the compounds according to the present disclosure as well as for the antibiotics in concurrent administration, taking into account factors well known in the art including type of subject being dosed, age, weight, sex and medical condition of the subject, the route of administration, the renal and hepatic function of the subject, the desired effect, the particular compound according to the present invention employed and the tolerance of the subject to it. Dosages of all antimicrobial agents should be adjusted in patients with renal impairment or hepatic insufficiency, due to the reduced metabolism and/or excretion of the drugs in patients with these conditions. Doses in children should also be reduced, generally according to body weight. The total daily dose of a derivative according to the present invention administered to a human or an animal can vary, for example, in amounts from 0.1 to 100 mg per kg body weight, in some cases, from 0.25 to 25 mg per kg body weight.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-51 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A conjugate comprising: a) an antimicrobial agent; and b) a hydrophilic polymer, wherein the antimicrobial agent is covalently linked, directly or via a linker, to the hydrophilic polymer.

Aspect 2. The conjugate of Aspect 2, wherein the conjugate exhibits reduced toxicity compared to the toxicity exhibited by the antimicrobial agent in unconjugated form.

Aspect 3. The conjugate of Aspect 3, wherein side effects induced by the conjugate are reduced relative to the side effects induced by the antimicrobial agent in unconjugated form.

Aspect 4. The conjugate of Aspect 1, wherein the antimicrobial agent is a polymyxin antibiotic, an aminoglycoside antibiotic, a cationic antimicrobial peptide, or a dibasic macrolide antibiotic.

Aspect 5. The conjugate of Aspect 4, wherein the polymyxin antibiotic is colistin, colistin sulfate, colistin methane-sulfonate, or a polymyxin derivative.

Aspect 6. The conjugate of Aspect 1, wherein the antimicrobial agent is an antibody specific for a microbial antigen.

Aspect 7. The conjugate of Aspect 1, wherein the antimicrobial agent is a polypeptide that enhances antimicrobial activity of an antibiotic.

Aspect 8. The conjugate of Aspect 7, wherein the polypeptide that enhances antimicrobial activity of an antibiotic is polymyxin B nonapeptide, NAB7061, or NAB741.

Aspect 9. The conjugate of Aspect 7, wherein the polypeptide that enhances antimicrobial activity of an antibiotic is a polymyxin derivative.

Aspect 10. The conjugate of Aspect 1, wherein the antimicrobial agent is an agent that facilitates entry of an antibiotic into a microbial cell.

Aspect 11. The conjugate of Aspect 11, wherein the hydrophilic polymer is poly(ethylene glycol) (PEG), poly(ethylene oxide) (PEO), poly(N-isopropylacrylamide) (PNIPAM), poly(2-oxazoline), polyethylenimine (PEI), poly(vinyl alcohol) (PVA), or poly(vinylpyrrolidone) (PVP).

Aspect 12. The conjugate of Aspect 12, wherein the hydrophilic polymer is a maltodextrin polymer.

Aspect 13. The conjugate of Aspect 12, wherein the maltodextrin polymer is maltotriose, maltotetraose, maltopentaose, maltohexaose, maltoheptaose, maltooctaose, maltononaose, or maltodecaose.

Aspect 14. The conjugate of any one of Aspects 1-7, wherein the maltodextrin polymer comprises from 2 to 20,000 α(1→4)-linked D-glucose subunits.

Aspect 15. The conjugate of any one of Aspects 1-14, wherein the polymer has a molecular weight of from about 0.5 Da to about 2000 kDa.

Aspects 16. The conjugate of any one of Aspects 1-15, wherein the antimicrobial agent is conjugated to the hydrophilic polymer via a cleavable linker.

Aspect 17. The conjugate of Aspect 16, wherein the cleavable linker is a proteolytically cleavable linker.

Aspect 18. The conjugate of Aspect 17, wherein the cleavable linker is a self-immolative linker.

Aspect 19. The conjugate of Aspect 18, wherein the self-immolative linker is cleavable by a thiol.

Aspect 20. The conjugate of Aspect 19, wherein the thiol is glutathione.

Aspect 21. The conjugate of Aspect 17, wherein the cleavable linker is a water-hydrolyzable linker.

Aspect 22. The conjugate of any one of Aspects 1-21, wherein the molar ratio of antimicrobial agent to hydrophilic polymer is from 1:1 to 100:1.

Aspect 23. A pharmaceutical composition comprising: the conjugate of any one of claims 1-22; and a pharmaceutically acceptable excipient.

Aspect 24. The composition of Aspect 23, wherein the pharmaceutical composition is a liquid composition.

Aspect 25. The composition of Aspect 23, wherein the composition is an aerosol.

Aspect 26. The composition of Aspect 23, wherein the composition a gel, a semi-solid, or a solid.

Aspect 27. The composition of any one of Aspects 23-26, wherein the conjugate is present in the composition in a concentration of from 0.01 μg/ml to 200 mg/ml.

Aspect 28. A method of inhibiting growth of a bacterium, the method comprising contacting the bacterium with the conjugate of any one of Aspects 1-22 or the composition of any one of claims 23-27.

Aspect 29. The method of Aspect 29, wherein the bacterium is a gram-negative bacterium.

Aspect 30. The method of Aspect 29, wherein the bacterium is a gram-positive bacterium.

Aspect 31. The method of any one of Aspects 28-30, wherein the bacterium is resistant to a carbapenem antibiotic.

Aspect 32. The method of any one of Aspects 28-30, wherein the bacterium is resistant to more than one antibiotic.

Aspect 33. The method of Aspect 28, wherein the bacterium is Pseudomonas aeruginosa, Klebsiella pneumoniae, Acinetobacter baumannii, Escherichia coli, or Staphylococcus aureus.

Aspect 34. The method of any one of Aspects 28-33, wherein the minimum inhibitory concentration of the conjugate is from about 0.01 μg/ml to 10 μg/ml of unconjugated antimicrobial agent equivalents.

Aspect 35. A method of treating a bacterial infection in an individual, the method comprising administering to the individual an effective amount of the conjugate of any one of claims 1-22 or the composition of any one of Aspects 23-27.

Aspect 36. The method of Aspect 24, wherein the conjugate is administered in a dose of from about 1 mg/kg per day to about 100 mg/kg per day, wherein the dose is based on the amount of equivalents of unconjugated antimicrobial agent.

Aspect 37. The method of Aspect 35 or 36, wherein the conjugate is administered via oral administration.

Aspect 38. The method of Aspect 35 or 36, wherein the conjugate is administered via pulmonary administration.

Aspect 39. The method of Aspect 35 or 36, wherein the conjugate is administered via inhalational administration.

Aspect 40. The method of Aspect 35 or 36, wherein the conjugate is administered via intranasal administration.

Aspect 41. The method of Aspect 35 or 36, wherein the conjugate is administered via mucosal administration.

Aspect 42. The method of Aspect 35 or 36, wherein the conjugate is administered via topical administration.

Aspect 43. The method of Aspect 35 or 36, wherein the conjugate is administered via ocular administration.

Aspect 44. The method of Aspect 35 or 36, wherein the conjugate is administered via intravenous administration.

Aspect 45. The method of Aspect 35 or 36, wherein the conjugate is administered via subcutaneous administration.

Aspect 46. The method of any one of Aspect 35-45, further comprising administering at least one additional therapeutic agent.

Aspect 47. The method of Aspect 46, wherein the at least one additional therapeutic agent is an antibiotic that is different from the antimicrobial agent in the conjugate.

Aspect 48. The method of Aspect 47, wherein the antibiotic is rifampicin, rifabutin, rifalazil, rifapentine, rifaximin, oxacillin, methicillin, ampicillin, cloxacillin, carbenicillin, piperacillin, tricarcillin, flucloxacillin, nafcillin, azithromycin, clarithromycin, erythromycin, telithromycin, cethromycin, solithromycin, aztreonam, BAL30072, meropenem, doripenem, imipenem, ertapenem, biapenem, tomopenem, panipenem, tigecycline, omadacycline, eravacycline, doxycycline, minocycline, ciprofloxacin, levofloxacin, moxifloxacin, delafloxacin, fusidic acid, novobiocin, teichoplanin, telavancin, dalbavancin, or oritavancin, or a pharmaceutically acceptable salt or solvates of same.

Aspect 49. The method of any one of Aspect 35-48, wherein the individual is a human.

Aspect 50. The method of any one of Aspect 35-48, wherein the individual is a non-human animal.

Aspect 51. The method of Aspect 50, wherein the non-human animal is a mammal.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c, subcutaneous(ly); and the like.

### Example 1

A technology for targeting colistin to bacteria based on the maltodextrin family of oligosaccharides has been developed. The new compounds are composed of maltodextrins conjugated to colistin and are termed them the CMCs (Colistin-Maltodextrin Conjugate). The CMCs have high specificity for bacteria because they should bind bacteria via the maltodextrin transport pathway, but are not internalized by mammalian cells because they lack maltodextrin transporters. CMC can have a wider therapeutic window than free colistin, allowing it to treat drug resistant bacteria.

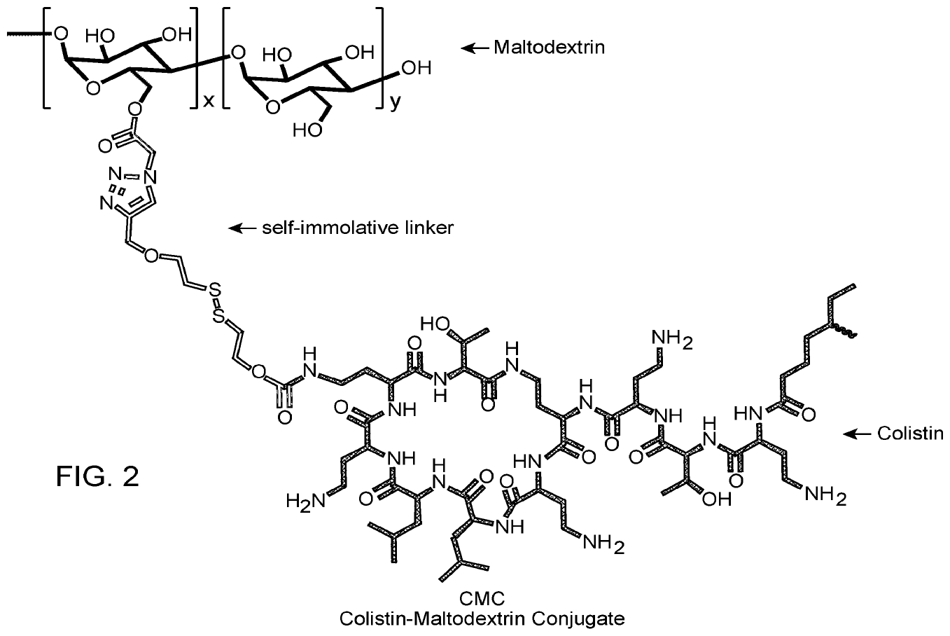

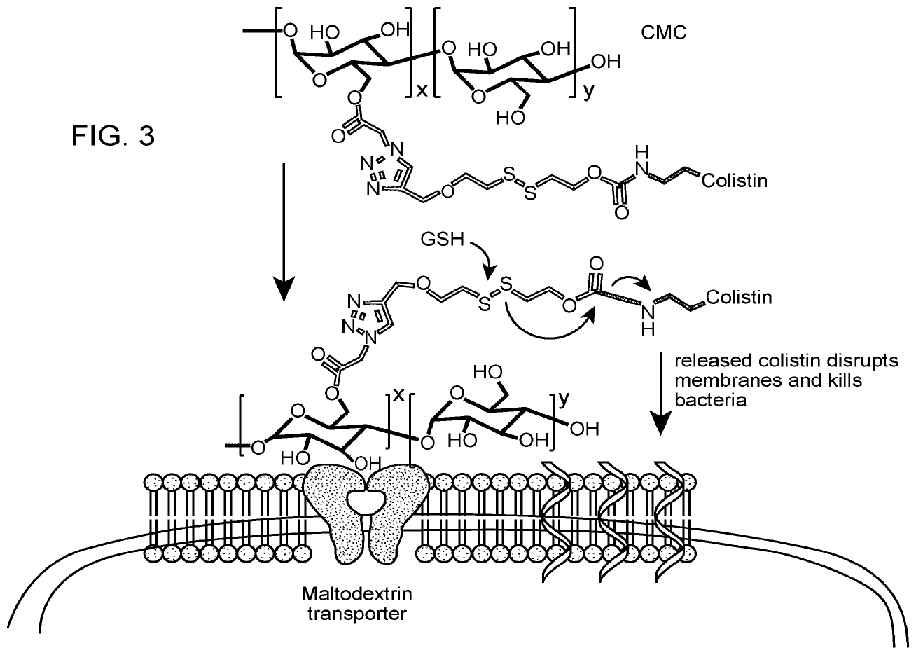

The experiments focus on targeting colistin to bacteria via the maltodextrin transporter. These experiments are based on the recent demonstration that maltohexaose conjugated fluorescent dyes can selectively target bacteria in vivo and in vitro. FIG. 2 shows a representative example of an imaging experiment done with MDP-2 in rats infected with E. coli (10⁷CFUs), and demonstrates that MDP-2 accumulated in the infected muscle tissue, and generated a 26-fold increase in fluorescence between infected and uninfected muscle tissues. Importantly, MDP-2 did not accumulate in the bacterial microflora, due to the impermeability of glucose oligomers to the lumen of intestinal tissues. MDP-2 was also efficiently cleared from all the major organs, indicating that maltohexaose-based targeting could potentially be used for imaging a wide range of tissues and also for targeting antibiotics to bacteria with specificity. Finally, it was demonstrated that MDP-1, a conjugate of maltohexaose with perylene was efficiently internalized by E. coli in either the planktonic or biofilm state.

A strategy for targeting bacteria based on targeting the maltodextrin transporter has been developed using CMCs, which target bacteria. In particular, the maltodextrin transporter has high specificity for bacteria and a robust uptake capacity (Km=200 μM), and its ligand, the maltodextrins have extremely low toxicity and are membrane impermeable. Maltodextrin targeting therefore has the potential to influence all aspects of infectious diseases, ranging from the development of new diagnostics to new therapeutics. The current technology shows that maltodextrin transport pathway can be used for targeting therapeutics to bacteria.

Colistin will be conjugated to maltodextrins, which is termed as CMC (Colistin-Maltodextrin Conjugate), via a self-immolative linker that can be cleaved by glutathione (GSH) or other thiols in serum or in bacteria. The CMC is designed to be initially cleaved by amylases in the serum, and generate maltodextrins 2-12 units in length, conjugated to colistin, which then target bacteria, via the maltodextrin transporter. After binding the cell surface of the bacteria, thiols in the serum cleave the immolative linker and release unmodified colistin, which then causes bacterial cell death (FIG. 3). The experiments in this disclosure focus on improving the treatment of drug resistant bacterial infections by targeting colistin to gram negative bacteria with maltodextrins. A conjugate as described herein will a significant impact on the treatment of gram negative infections, because such a conjugate will allow colistin to be given to patients at doses that can treat drug resistant infections with low toxicity.

### Synthesis of Colistin-Maltodextrin Conjugates (CMC)

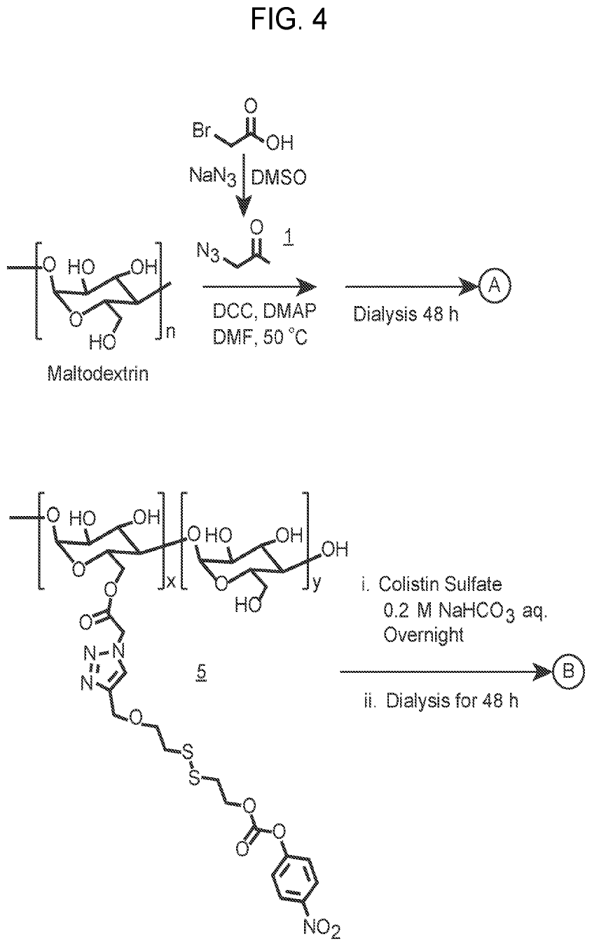

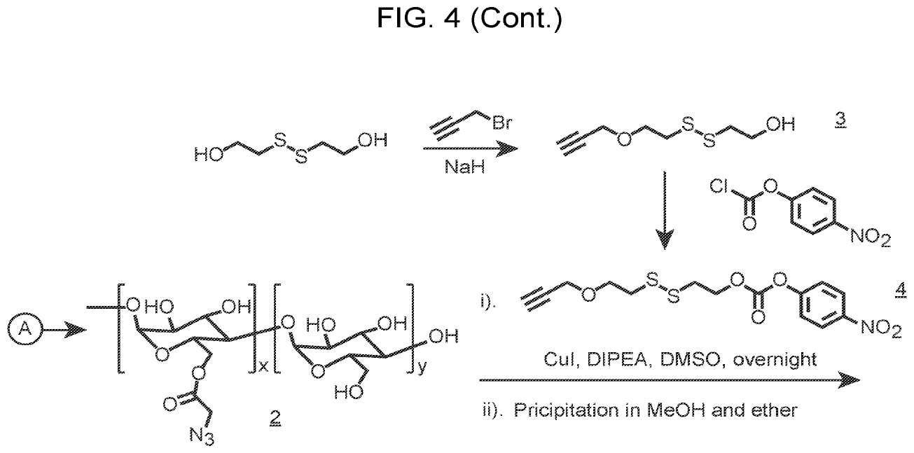

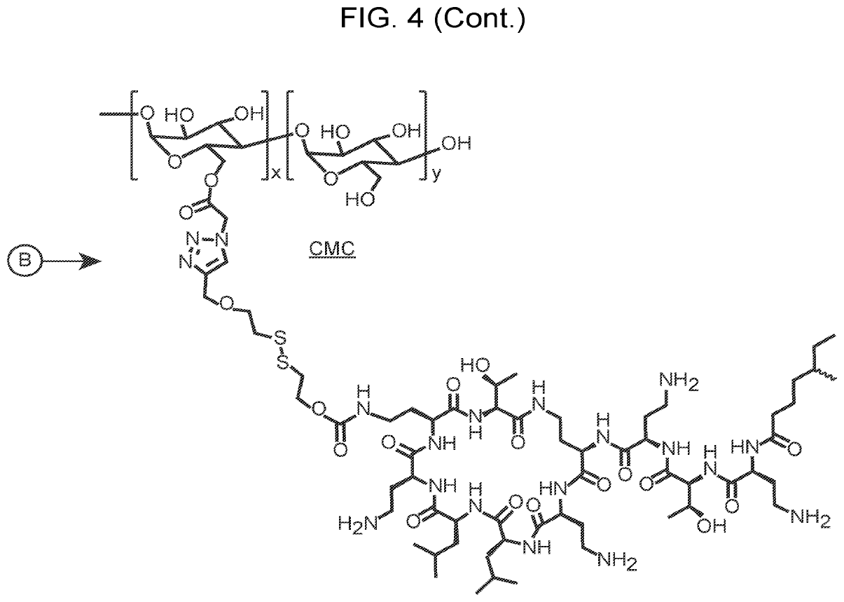

CMC is designed to be hydrolyzed by amylases in the serum, bind maltodextrin transporters on bacteria and then undergo disulfide reduction and release free colistin, which then causes toxicity to bacteria. The synthesis of CMC is shown in FIG. 4, and was accomplished in 5 steps. Maltodextrin of 28,000 molecular weight was coupled to azido acetic acid and then clicked onto the heterobifunctional cross-linker (4) that contains a terminal alkyne and a para-nitrophenyl activated hydroxyl. Importantly, the compound 4 also contains a self-immolative disulfide linkage that can be cleaved in the presence of thiols such as glutathione (GSH). The para-nitrophenyl activated maltodextrin was then conjugated to colistin and purified via dialysis. A detailed synthetic procedure is described below.

### Synthesis of Azido Acetic Acid (1):

Sodium azide (4.91 gm, 0.0755 mol) was suspended in DMSO (100 ml) and stirred for 1.5 h at room temperature to get a clear yellow solution. Bromoacetic acid (5 g, 0.0359 mol) dissolved in DMSO (10 ml) was added dropwise to sodium azide in DMSO. The reaction mass was allowed to react at room temperature overnight. Reaction mixture was diluted with water and was acidified with HCl. Product was extracted with ethyl acetate thrice and the combined organic layers were washed with brine, dried over anhydrous sodium sulfate and concentrated to give the desired product as a pale yellow oil (2.5 gm, 65%). 1H NMR (300 MHz, CDCl3): δ 3.75 (s, 2H).

### Synthesis of Azide Functionalized Maltodextrin (2):

Maltodextrin (Mw ˜28000 Da) (1 g, 3.5×10⁵ mol) was suspended in anhydrous DMF (30 ml). Azido acetic acid (0.108 g, 7.1×10⁵ mol) and DCC (0.22 g, 7.1×10⁵ mol) were added followed by DMAP (0.047 g, 9×10⁵ mol). The reaction mixture was purged with nitrogen and stirred at 50° C. overnight. The resultant reaction mass was poured into rapidly stirring diethyl ether (300 ml) and the precipitated solid was stirred for 6 hr. The ether was removed by filtration under vacuum and the solid was dissolved in minimum amount of distilled water (8 ml) and dialyzed (MWCO 10 KDa) against 4×2 L of distilled water for 24 h. The resulting solution was lyophilized to yield the azide functionalized maltodextrin 2 (400 mg, 40%).

### Synthesis of Linker Intermediate Compound (3):

Sodium hydride (0.31 g, 12.5 mmol) was added slowly at 0° C. was added to a solution of 2-hydroxyethyl disulfide (2.67 g, 16.8 mmol) in THF (26 mL). The solution was stirred constantly for 30 min at room temperature until gas was no longer generated. 0.73 mL (8.4 mmol) of propargyl bromide was then added in a dropwise fashion at 0° C. The reaction mixture was stirred overnight at room temperature. The mixture was filtered, and the solvent was removed by evaporation under a reduced pressure at 50° C. The crude product was purified by column chromatography to give yellow oil (0.82 g, 54%).

### Synthesis of Disulfide Linker (4):

Compound 3 (0.4 g, 2.09 mmol) and 4-nitrophenyl carbonochloridate (0.63 g, 3.13 mmol) was dissolved in 15 mL dry acetonitrile. Pyridine (0.4 mL) was added and the reaction mixture was stirred at room temperature overnight under the nitrogen atmosphere. The mixture was evaporated to until it was dried and the resulting crude was purified by chromatography, giving the product as pale yellow oil (0.43 g, 58%).

### Synthesis of Disulfide Linker Functionalized Maltodextrin (5):

Alkyne functionalized disulfide linker 4 (115 mg, 3.2×10−4 mol), CuI (8.1 mg, 4.2×10⁵ mol) followed by DIPEA (0.55 ml, 3.2×10⁴ mol) were added to Compound 2 (0.3 g, 1.07×10⁵ mol) dissolved in DMSO (20 ml) under a nitrogen atmosphere. The resultant reaction mixture was stirred at room temperature overnight. The polymer was precipitated in MeOH (200 ml) and stirred for 4 h followed by filtration and precipitation, which was repeated two times. The polymer was dried under vacuum yielded the compound 4 (200 mg, 60%).

### Synthesis of Colistin-Maltodextrin Conjugate (CMC):

Linker functionalized maltodextrin (5) (0.1 gm, 3.5×10−6 mol) dissolved in DMSO (1 ml) was added in dropwise fashion to the Colistin sulfate (0.134 gm, 1.07×10⁴ mol) dissolved in water (15 ml), followed by NaHCO₃(4.4 mg, 1.07×10−4 mol). The reaction was allowed to react at room temperature overnight. The resulting polymer was dialyzed (MWCO 10 KDa) against 4×1 L distilled water for 48 h and freeze dried. The solution resulted the maltodextrin-colistin conjugate (CMC) (30 mg, 30%).

### Colistin-Maltodextrin Conjugate (CMC) is Effective at Inhibiting the Growth of Bacteria:

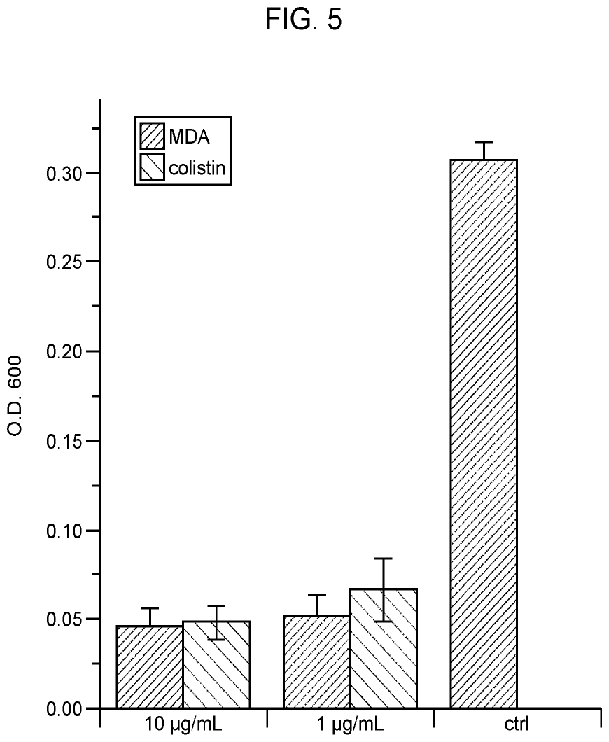

Experiments were performed to determine if CMC could effectively kill bacteria. Studies were performed on E. coli with the first CMC. CMC or free colistin was incubated with E. coli (BL21 strain) at 37° C. and the toxicity was measured via O.D. 600 after overnight culture. As shown in FIG. 5, CMC can effectively kill E. coli at 1 μg/mL of colistin equivalents.

### The MICs of the CMC Against a Panel of Gram-Negative Bacteria:

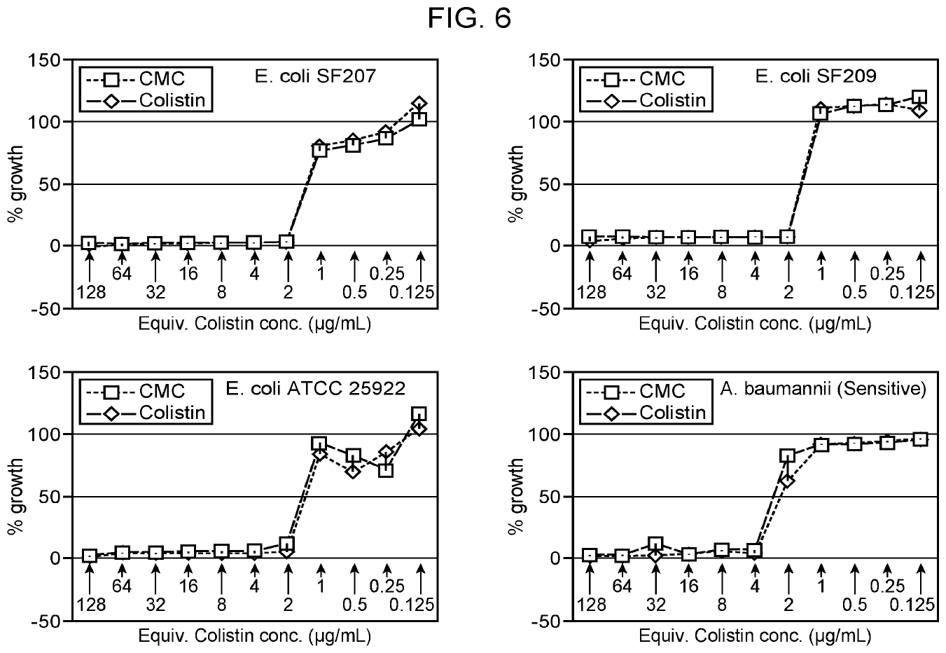

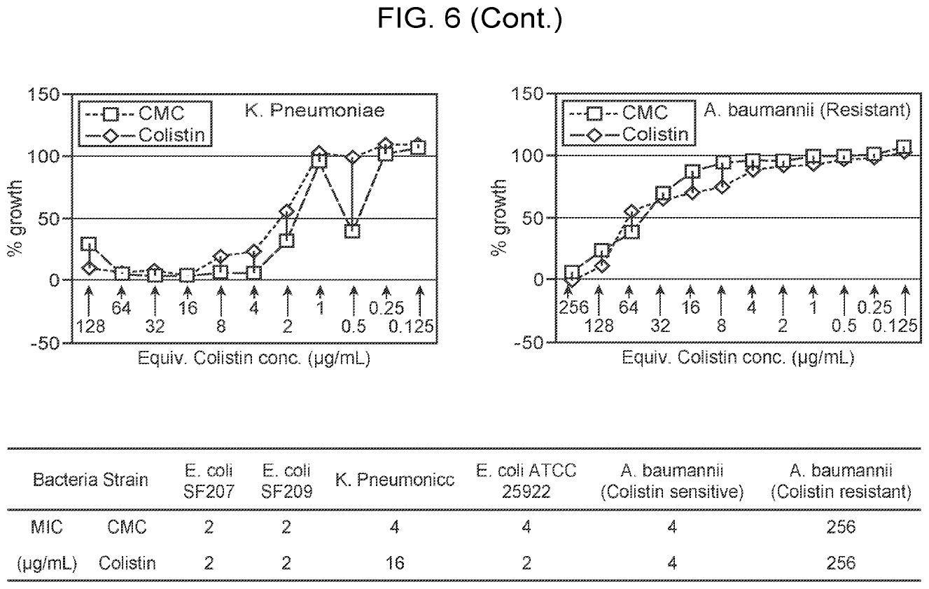

Experiments were performed to determine the MICs of CMC and free colistin against a panel of pathogenic gram-negative bacteria strains including hospital E. coli samples 207 and 209, K. pneumonic, E. coli ATCC 25922, A. baumannii (Colistin sensitive) and A. baumannii (Colistin Resistant). As shown in FIG. 6, CMC exhibited similar level of MICs to free colistin, which included very low MICs for all tested gram-negative bacteria strains except the colistin resistant one. CMC can therefore release free colistin in its active form.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

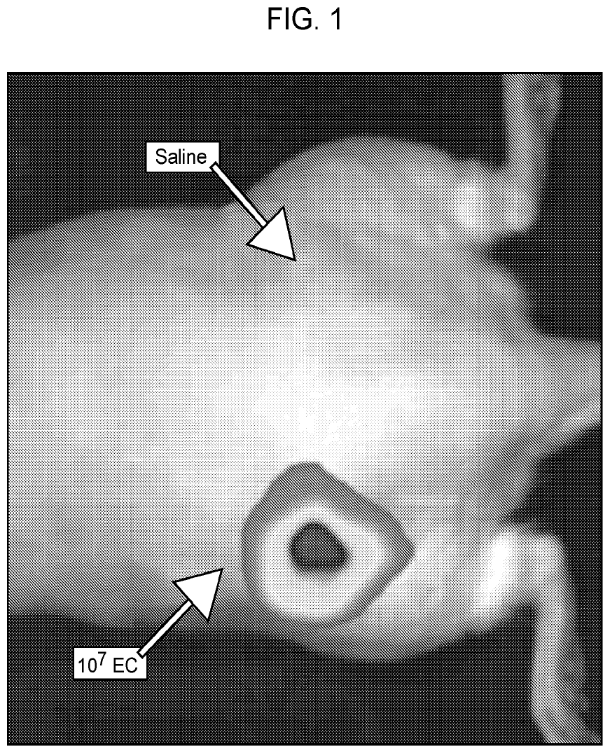

FIG. 1 shows that MDP-2 can image E. coli in vivo. 10⁷ CFUs of E. coli were injected into the left thigh muscles of rats, and the right thigh muscle was injected with saline as a control. After 1 hour, MDP-2 (280-350 μL of 1 mM MDP-2 in PBS) was injected into the rats via the jugular vein.

FIG. 2 shows the chemical structure of Colistin-Maltodextrin Conjugate (CMC).

FIG. 3 shows a schematic illustration of targeted antimicrobial effect of Colistin-Maltodextrin Conjugate.

FIG. 4 shows the synthetic route to CMC.

FIG. 5 shows an evaluation of the antimicrobial effect of CMC and its MICs. CMC can inhibit E. coli growth. CMC or colistin was mixed with E. coli and the O.D. at 600 nm was measured. CMC can inhibit E. coli growth at a concentration of 1 μg/mL of colistin equivalents.

FIG. 6 shows MICs of colistin and CMC against various strains of bacteria. CMC exhibited high antibiotic effect by showing the similar MICs to that of Colistin for all six bacteria strains. MIC of CMC was presented as the μg·mL⁻¹ of equivalent Colistin content.

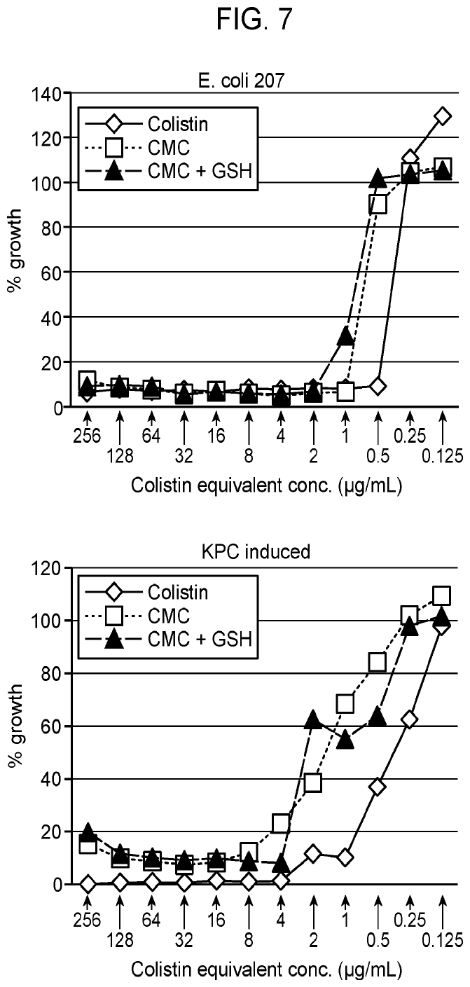

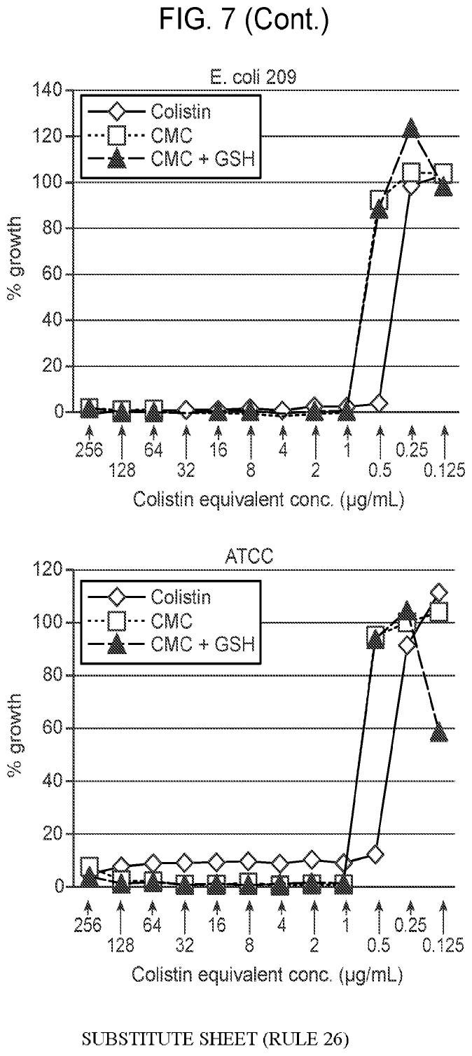

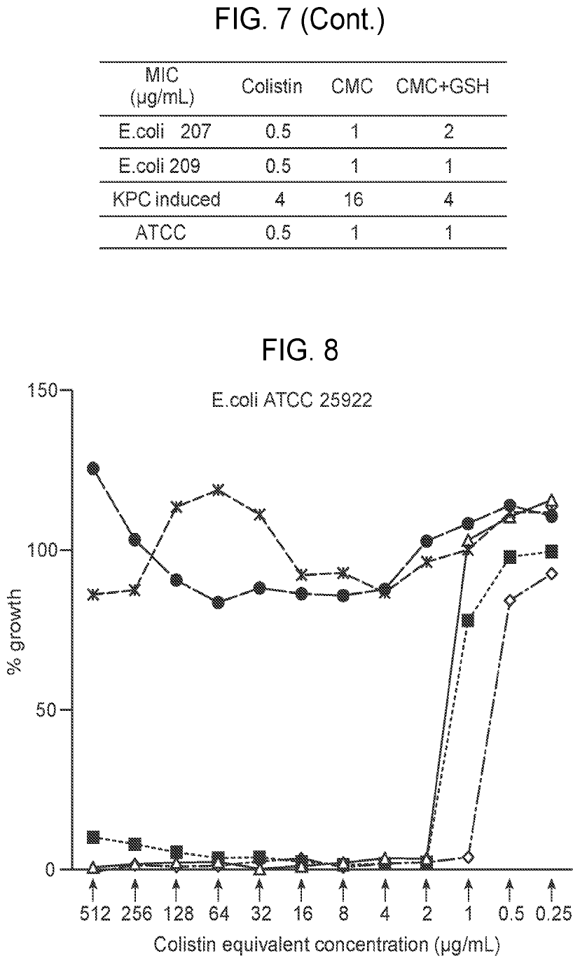

FIG. 7 shows MICs of colistin, CMC, or a combination of CMC+GSH against various strains of bacteria. “ATCC” refers to E. coli ATCC 25922. “KPC”: Klebsiella pneumoniae carbapenemase.

FIG. 8 shows MIC of maltodextrin, maltodextrin-linker, and CMC without tris(2-carboxyethyl)phosphine (TCEP) and spinfiltration. The data demonstrate that the active component in the colistin-maltodextrin conjugate is colistin. The maltodextrin and maltodextrin intermediates are not toxic to bacteria. However, if the colistin-maltodextrin is reduced with TCEP, the colistin component is released from colistin-maltodextrin; after treatment with TCEP, the colistin can be isolated by spin filtration.

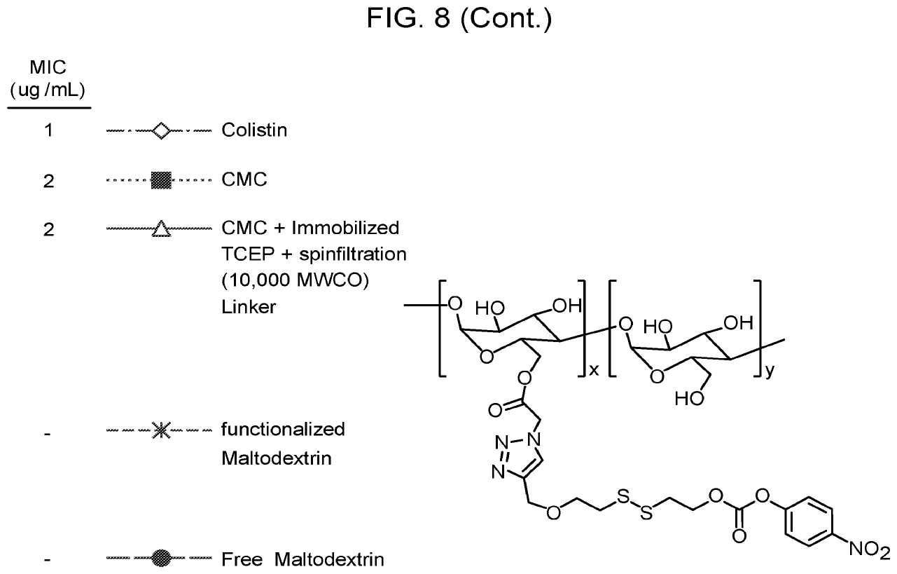

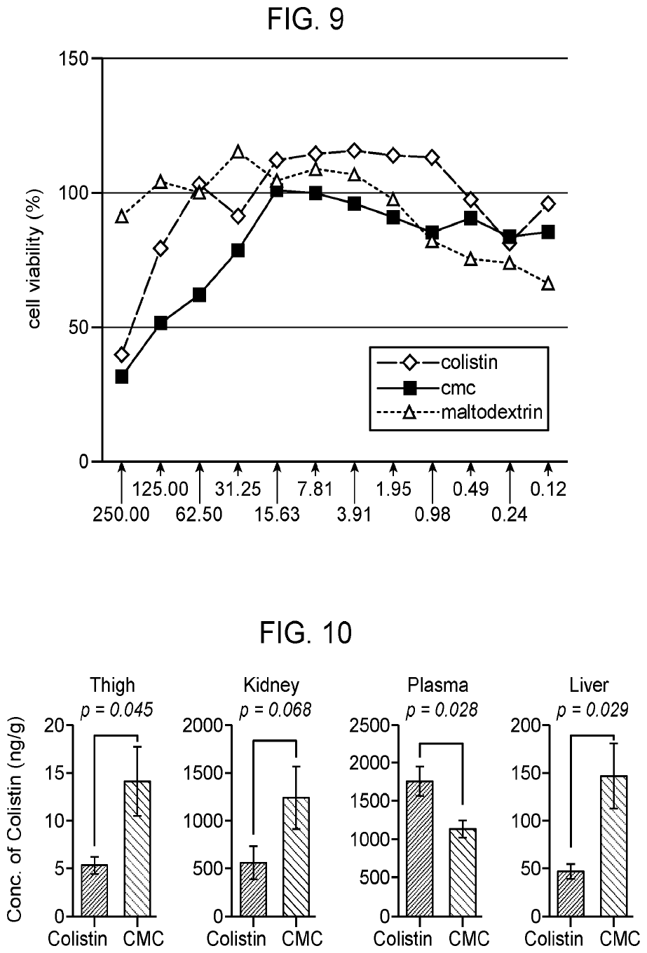

FIG. 9 depicts toxicity of CMC to mammalian cells, as shown by cell viability after contacting the cells with various amounts of colistin (free colistin), CMC, or maltodextrin. The units on the x-axis are μg/mL.

FIG. 10 depicts biodistribution of colistin after injection of CMC into infected mouse. CMC can target colistin to infected thigh muscles. The concentration of colistin in various organs (thigh, kidney, plasma, and liver) 30 minutes after the injection of free colistin or CMC (1 mg/kg colistin equivalent) in P. aeruginosa infected mice was determined. The error bar indicates the standard error, n=3. Each mouse was infected by injecting 5×10⁵ CFU/thigh of P. aeruginosa into the thigh muscles. The colistin or CMC treatment was performed 2 hours after the bacteria injection.

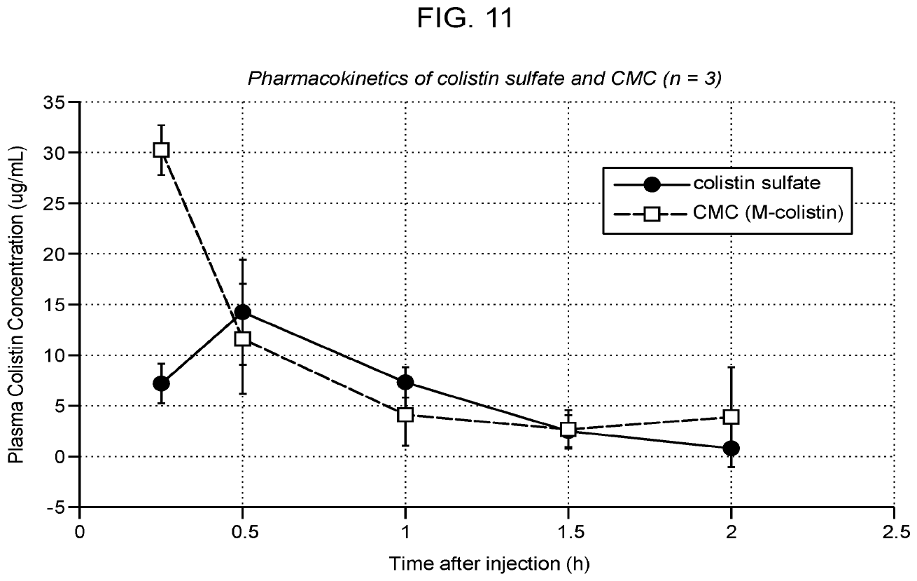

FIG. 11 depicts pharmacokinetics of colistin sulfate and CMC. 1 mg/ml of colistin and 25 mg/ml of M-colistin were used for the experiments. 1 mg/kg of colistin sulfate or 25 mg/kg of CMC (M-colistin) were injected via the tail vein. The mice (thigh infected) were sacrificed 0.25, 0.5, 1, 1.5 or 2 hours after the injection. The plasma samples were diluted to 1/100 for ELISA. (5 μl of plasma+395 μl of 1×extraction buffer+100 ul of acetonitrile). n=3.

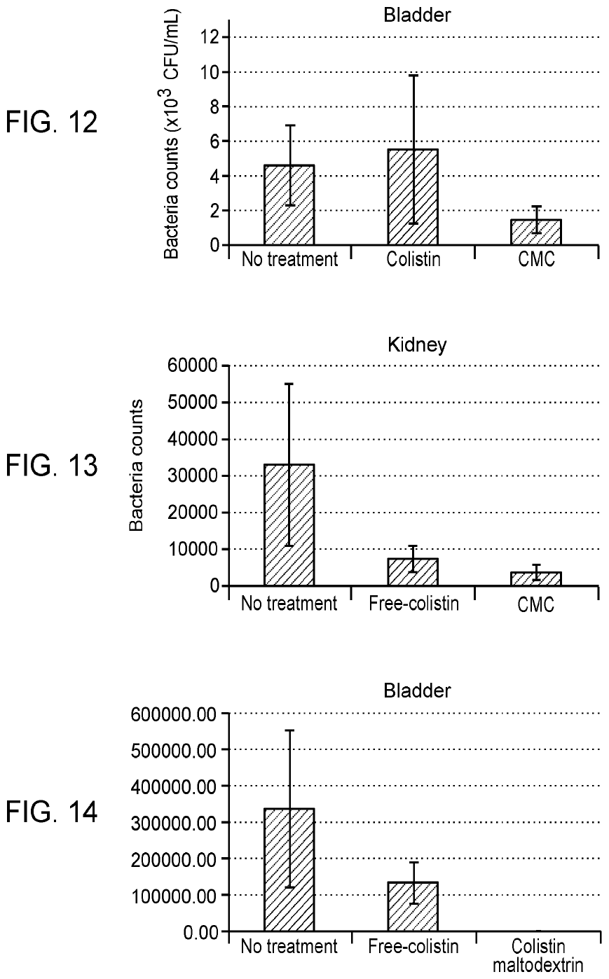

FIG. 12 depicts the effect of CMC on urinary tract infection (UTI). Mice were infected with E. coli and were treated with colistin, CMC or no treatment for 3 days. The mice were sacrificed and their bladders were harvested and the CFU count in the bladders was determined. Mice treated with CMC have a lower CFU count than mice treated with free colistin. The data show that CMC can effectively treat E. coli UTIs and dramatically improves the efficacy of colistin.

FIG. 13 depicts the effect of CMC on UTI. Mice were infected with E. coli and were treated with colistin, CMC or no treatment for 3 days. The mice were sacrificed and their kidneys were harvested and the CFU count in the bladders was determined. Mice treated with CMC have a lower CFU count than mice treated with free colistin.

FIG. 14 depicts the effect of free colistin, or colistin-maltodextrin, on bacterial counts in the bladder.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A conjugate comprising:
a) an antimicrobial agent; and
b) a hydrophilic polymer,
wherein the antimicrobial agent is covalently linked, directly or via a linker, to the hydrophilic polymer.

2. The conjugate of claim 1, wherein the conjugate exhibits reduced toxicity compared to the toxicity exhibited by the antimicrobial agent in unconjugated form.

3. The conjugate of claim 1, wherein side effects induced by the conjugate are reduced relative to the side effects induced by the antimicrobial agent in unconjugated form.

4. The conjugate of claim 1, wherein the antimicrobial agent is a polymyxin antibiotic, an aminoglycoside antibiotic, a cationic antimicrobial peptide, or a dibasic macrolide antibiotic.

5. The conjugate of claim 4, wherein the polymyxin antibiotic is colistin, colistin sulfate, colistin methane-sulfonate, or a polymyxin derivative.

6. The conjugate of claim 1, wherein the antimicrobial agent is an antibody specific for a microbial antigen.

7. The conjugate of claim 1, wherein the antimicrobial agent is a polypeptide that enhances antimicrobial activity of an antibiotic.

8. The conjugate of claim 7, wherein the polypeptide that enhances antimicrobial activity of an antibiotic is polymyxin B nonapeptide, NAB7061, or NAB741.

9. The conjugate of claim 6, wherein the polypeptide that enhances antimicrobial activity of an antibiotic is a polymyxin derivative.

10. The conjugate of claim 1, wherein the antimicrobial agent is an agent that facilitates entry of an antibiotic into a microbial cell.

11. The conjugate of claim 1, wherein the hydrophilic polymer is poly(ethylene glycol) (PEG), poly(ethylene oxide) (PEO), poly(N-isopropylacrylamide) (PNIPAM), poly(2-oxazoline), polyethylenimine (PEI), poly(vinyl alcohol) (PVA), or poly(vinylpyrrolidone) (PVP).

12. The conjugate of claim 1, wherein the hydrophilic polymer is a maltodextrin polymer.

13. The conjugate of claim 11, wherein the maltodextrin polymer is maltotriose, maltotetraose, maltopentaose, maltohexaose, maltoheptaose, maltooctaose, maltononaose, or maltodecaose.

14. The conjugate of any one of claims 1-7, wherein the maltodextrin polymer comprises from 2 to 20,000 α(1→4)-linked D-glucose subunits.

15. The conjugate of any one of claims 1-14, wherein the polymer has a molecular weight of from about 0.5 Da to about 2000 kDa.

16. The conjugate of any one of claims 1-15, wherein the antimicrobial agent is conjugated to the hydrophilic polymer via a cleavable linker.

17. The conjugate of claim 16, wherein the cleavable linker is a proteolytically cleavable linker.

18. The conjugate of claim 17, wherein the cleavable linker is a self-immolative linker.

19. The conjugate of claim 18, wherein the self-immolative linker is cleavable by a thiol.

20. The conjugate of claim 19, wherein the thiol is glutathione.

21. The conjugate of claim 17, wherein the cleavable linker is a water-hydrolyzable linker.

22. The conjugate of any one of claims 1-21, wherein the molar ratio of antimicrobial agent to hydrophilic polymer is from 1:1 to 100:1.

23. A pharmaceutical composition comprising:
a) the conjugate of any one of claims 1-22; and
b) a pharmaceutically acceptable excipient.

24. The composition of claim 23, wherein the pharmaceutical composition is a liquid composition.

25. The composition of claim 23, wherein the composition is an aerosol.

26. The composition of claim 23, wherein the composition a gel, a semi-solid, or a solid.

27. The composition of any one of claims 23-26, wherein the conjugate is present in the composition in a concentration of from 0.01 μg/ml to 200 mg/ml.

28. A method of inhibiting growth of a bacterium, the method comprising contacting the bacterium with the conjugate of any one of claims 1-22 or the composition of any one of claims 23-27.

29. The method of claim 29, wherein the bacterium is a gram-negative bacterium.

30. The method of claim 29, wherein the bacterium is a gram-positive bacterium.

31. The method of any one of claims 28-30, wherein the bacterium is resistant to a carbapenem antibiotic.

32. The method of any one of claims 28-30, wherein the bacterium is resistant to more than one antibiotic.

33. The method of 28, wherein the bacterium is Pseudomonas aeruginosa, Klebsiella pneumoniae, Acinetobacter baumannii, Escherichia coli, or Staphylococcus aureus.

34. The method of any one of claims 28-33, wherein the minimum inhibitory concentration of the conjugate is from about 0.01 μg/ml to 10 μg/ml of unconjugated antimicrobial agent equivalents.

35. A method of treating a bacterial infection in an individual, the method comprising administering to the individual an effective amount of the conjugate of any one of claims 1-22 or the composition of any one of claims 23-27.

36. The method of claim 24, wherein the conjugate is administered in a dose of from about 1 mg/kg per day to about 100 mg/kg per day, wherein the dose is based on the amount of equivalents of unconjugated antimicrobial agent.

37. The method of claim 35 or 36, wherein the conjugate is administered via oral administration.

38. The method of claim 35 or 36, wherein the conjugate is administered via pulmonary administration.

39. The method of claim 35 or 36, wherein the conjugate is administered via inhalational administration.

40. The method of claim 35 or 36, wherein the conjugate is administered via intranasal administration.

41. The method of claim 35 or 36, wherein the conjugate is administered via mucosal administration.

42. The method of claim 35 or 36, wherein the conjugate is administered via topical administration.

43. The method of claim 35 or 36, wherein the conjugate is administered via ocular administration.

44. The method of claim 35 or 36, wherein the conjugate is administered via intravenous administration.

45. The method of claim 35 or 36, wherein the conjugate is administered via subcutaneous administration.

46. The method of any one of claims 35-45, further comprising administering at least one additional therapeutic agent.

47. The method of claim 46, wherein the at least one additional therapeutic agent is an antibiotic that is different from the antimicrobial agent in the conjugate.

48. The method of claim 47, wherein the antibiotic is rifampicin, rifabutin, rifalazil, rifapentine, rifaximin, oxacillin, methicillin, ampicillin, cloxacillin, carbenicillin, piperacillin, tricarcillin, flucloxacillin, nafcillin, azithromycin, clarithromycin, erythromycin, telithromycin, cethromycin, solithromycin, aztreonam, BAL30072, meropenem, doripenem, imipenem, ertapenem, biapenem, tomopenem, panipenem, tigecycline, omadacycline, eravacycline, doxycycline, minocycline, ciprofloxacin, levofloxacin, moxifloxacin, delafloxacin, fusidic acid, novobiocin, teichoplanin, telavancin, dalbavancin, or oritavancin, or a pharmaceutically acceptable salt or solvates of same.

49. The method of any one of claims 35-48, wherein the individual is a human.

50. The method of any one of claims 35-48, wherein the individual is a non-human animal.

51. The method of claim 50, wherein the non-human animal is a mammal.


SEQUENCE LISTING (42 sequences)

SEQ ID NO: 1 - protein, 6 aa, Artificial Sequence
  synthetic sequence
  MISC_FEATURE (2)..(2): Xaa at position 2 may be Leu or Gln.
PXGMTS

SEQ ID NO: 2 - protein, 5 aa, Artificial Sequence
  synthetic sequence
  MISC_FEATURE (2)..(2): Xaa at position 2 may be Leu or Gln.
PXGMT

SEQ ID NO: 3 - protein, 7 aa, Artificial Sequence
  synthetic sequence
ENLYFQS

SEQ ID NO: 4 - protein, 7 aa, Artificial Sequence
  synthetic sequence
ENLYFQY

SEQ ID NO: 5 - protein, 7 aa, Artificial Sequence
  synthetic sequence
ENLYFQL

SEQ ID NO: 6 - protein, 5 aa, Artificial Sequence
  synthetic sequence
DDDDK

SEQ ID NO: 7 - protein, 4 aa, Artificial Sequence
  synthetic sequence
LVPR

SEQ ID NO: 8 - protein, 6 aa, Artificial Sequence
  synthetic sequence
LVPRGS

SEQ ID NO: 9 - protein, 8 aa, Artificial Sequence
  synthetic sequence
LEVLFQGP

SEQ ID NO: 10 - protein, 10 aa, Artificial Sequence
  synthetic sequence
CGLVPAGSGP

SEQ ID NO: 11 - protein, 12 aa, Artificial Sequence
  synthetic sequence
SLLKSRMVPNFN

SEQ ID NO: 12 - protein, 12 aa, Artificial Sequence
  synthetic sequence
SLLIARRMPNFN

SEQ ID NO: 13 - protein, 12 aa, Artificial Sequence
  synthetic sequence
SKLVQASASGVN

SEQ ID NO: 14 - protein, 12 aa, Artificial Sequence
  synthetic sequence
SSYLKASDAPDN

SEQ ID NO: 15 - protein, 12 aa, Artificial Sequence
  synthetic sequence
RPKPQQFFGLMN

SEQ ID NO: 16 - protein, 12 aa, Artificial Sequence
  synthetic sequence
SLRPLALWRSFN

SEQ ID NO: 17 - protein, 12 aa, Artificial Sequence
  synthetic sequence
SPQGIAGQRNFN

SEQ ID NO: 18 - protein, 14 aa, Artificial Sequence
  synthetic sequence
DVDERDVRGFASFL

SEQ ID NO: 19 - protein, 12 aa, Artificial Sequence
  synthetic sequence
SLPLGLWAPNFN

SEQ ID NO: 20 - protein, 12 aa, Artificial Sequence
  synthetic sequence
SLLIFRSWANFN

SEQ ID NO: 21 - protein, 12 aa, Artificial Sequence
  synthetic sequence
SGVVIATVIVIT

SEQ ID NO: 22 - protein, 12 aa, Artificial Sequence
  synthetic sequence
SLGPQGIWGQFN

SEQ ID NO: 23 - protein, 12 aa, Artificial Sequence
  synthetic sequence
KKSPGRVVGGSV

SEQ ID NO: 24 - protein, 12 aa, Artificial Sequence
  synthetic sequence
PQGLLGAPGILG

SEQ ID NO: 25 - protein, 31 aa, Artificial Sequence
  synthetic sequence
HGPEGLRVGFYESDVMGRGHARLVHVEEPHT

SEQ ID NO: 26 - protein, 12 aa, Artificial Sequence
  synthetic sequence
GPQGLAGQRGIV

SEQ ID NO: 27 - protein, 12 aa, Artificial Sequence
  synthetic sequence
GGSGQRGRKALE

SEQ ID NO: 28 - protein, 12 aa, Artificial Sequence
  synthetic sequence
SLSALLSSDIFN

SEQ ID NO: 29 - protein, 12 aa, Artificial Sequence
  synthetic sequence
SLPRFKIIGGFN

SEQ ID NO: 30 - protein, 12 aa, Artificial Sequence
  synthetic sequence
SLLGIAVPGNFN

SEQ ID NO: 31 - protein, 12 aa, Artificial Sequence
  synthetic sequence
FFKNIVTPRTPP

SEQ ID NO: 32 - protein, 7 aa, Artificial Sequence
  synthetic sequence
ENLYFQW

SEQ ID NO: 33 - protein, 7 aa, Artificial Sequence
  synthetic sequence
ENLYFQM

SEQ ID NO: 34 - protein, 7 aa, Artificial Sequence
  synthetic sequence
ENLYFQH

SEQ ID NO: 35 - protein, 7 aa, Artificial Sequence
  synthetic sequence
ENLYFQN

SEQ ID NO: 36 - protein, 7 aa, Artificial Sequence
  synthetic sequence
ENLYFQA

SEQ ID NO: 37 - protein, 7 aa, Artificial Sequence
  synthetic sequence
ENLYFQQ

SEQ ID NO: 38 - protein, 7 aa, Artificial Sequence
  synthetic sequence
DEVVECS

SEQ ID NO: 39 - protein, 10 aa, Artificial Sequence
  synthetic sequence
DEAEDVVECS

SEQ ID NO: 40 - protein, 11 aa, Artificial Sequence
  synthetic sequence
EDAAEEVVECS

SEQ ID NO: 41 - protein, 6 aa, Artificial Sequence
  synthetic sequence
PLFAAR

SEQ ID NO: 42 - protein, 11 aa, Artificial Sequence
  synthetic sequence
QQEVYGMMPRD